(19) United States
(12) Patent Application Publication
(10) Pub. No.: US 2025/0186373 A1
(43) Pub. Date: Jun. 12, 2025

(54) TARGETING AN EUKARYOTIC INITIATION FACTOR 2 ALPHA KINASE TO REGULATE TRANSLATION UNDER STRESS

(71) Applicant: The Johns Hopkins University, Baltimore, MD (US)

(72) Inventors: Jiou WANG, Baltimore, MD (US); YuNing LU, Baltimore, MD (US)

(21) Appl. No.: 18/846,017

(22) PCT Filed: Mar. 10, 2023
(86) PCT No.: PCT/US2023/064129

§ 371 Date: Sep. 11, 2024

Related U.S. Application Data
(60) Provisional application No. 63/318,997, filed on Mar. 11, 2022.

(51) Int. Cl. A61K 31/166; C07C 237/34; C07D 233/36

(52) U.S. Cl. CPC A61K 31/166; C07C 237/34; C07D 233/36

(57) ABSTRACT

The identification of a direct kinase of eukaryotic initiation factor 2 alpha (eIF2α), microtubule affinity-regulating kinase 2 (MARK2), which phosphorylates eIF2α in response to proteotoxic stress, is provided. Inhibitors of MARK2 and their use in treating neurodegenerative disease, including Alzheimer's disease, Parkinson disease, Creutzfeldt-Jakob disease, Huntington's disease, frontotemporal dementia (FTD), and amyotrophic lateral sclerosis (ALS), also are disclosed.

### STATEMENT OF GOVERNMENTAL INTEREST

This invention was made with government support under grant NS074324 awarded by the National Institute of Health. The government has certain rights in the invention.

### BACKGROUND

To maintain a state of fitness during stress, cells have evolved exquisite stress response programs that sense potentially harmful situations and make the necessary adaptations at the molecular and cellular levels. Stress signaling pathways are frequently mediated by protein kinases and phosphorylation substrates, whose specificity is determined by their interactions with temporal and spatial regulations. Miller and Turk, 2018. Proteins are responsible for most cellular functions, and the maintenance of protein homeostasis is required for the survival of cells, especially under stress conditions.

A key regulation of protein homeostasis occurs at the level of protein synthesis or translation. The first step in translation requires eukaryotic initiation factor 2 (eIF2), which is regulated by phosphorylation of serine 51 (⁵¹S) of its alpha subunit (eIF2α), with increased phosphorylation resulting in global attenuation of the translation of most transcripts and enhanced translation of select transcripts encoding stress response-related proteins. The phosphorylation of eIF2α is the central step during the integrated stress response, which allows cells to react to various types of stimuli by regulating translation. Holcik and Sonenberg, 2005.

To date, four kinases have been found to phosphorylate eIF2α in response to various stressors: protein kinase R (PKR), activated by double-stranded RNA, Feng et al., 1992; Prostko et al., 1995; PKR-like ER-resident kinase (PERK), responding to endoplasmic reticulum (ER) stress, Harding et al., 1999; heme-regulated eIF2α kinase (HRI), induced by low levels of heme, Chen et al., 1991; Chen and London, 1995; and general control nonderepressible factor 2 kinase (GCN2), sensing amino acid deficiency. Dever, 1992. Among them, PERK is capable of sensing protein misfolding as part of the unfolded protein response originating in the ER lumen, Ron and Walter, 2007; Christianson and Ye, 2014; HRI is expressed in an erythroid cell-specific manner and reported to be a cytosolic sensor of protein misfolding that controls innate immune signaling. Yerlikaya et al., 2008; Abdel-Nour et al., 2019; Crosby et al., 1994.

No other kinase has been identified that phosphorylates eIF2α and controls translation in response to protein unfolding stress. Stresses associated with protein misfolding have formed a common theme in neurodegenerative diseases, including Alzheimer disease, Parkinson disease, Creutzfeldt-Jakob disease, Huntington disease, frontotemporal dementia (FTD), and amyotrophic lateral sclerosis (ALS). Prusiner, 2012; Balch et al., 2008.

Among them, ALS is characterized by progressive motor neuron degeneration, with approximately 10% of cases inherited in families and both its familial and sporadic forms linked to diverse genetic mutations. Renton et al., 2014. One of the central themes in ALS pathology is protein misfolding and aggregation. For example, proteinaceous inclusions that harbor misfolded proteins, including Cu/Zn superoxide dismutase (SOD1), have been found in both familial and sporadic ALS patients. Rosen et al., 1993; Neumann et al., 2006; Sreedharan et al., 2008; Bosco et al., 2010.

A large number of mutations in SOD1, responsible for 20% of all familial ALS, cause the protein to gain a heightened propensity to misfold and aggregate. Wang et al., 2003; Wang et al., 2002; Lindberg et al., 2005; Deng et al., 1993; Zhong et al., 2017. The contrast between wild-type (WT) and mutant SOD1 proteins, the former being highly stable and the latter prone to aggregation, makes SOD1 a sensitive molecular model for studying protein aggregation. Wang et al., 2009; Zhang and Zhu, 2006.

### SUMMARY

In some aspects, the presently disclosed subject matter provides a compound of formula (I):

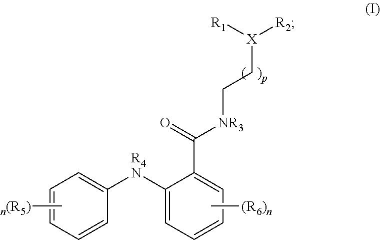

wherein:
   m is an integer selected from 0, 1, 2, 3, 4, and 5;
   n is an integer selected from 0, 1, 2, 3, and 4;
   p is an integer selected from 0, 1, 2, 3, 4, 5, 6, and 7;
   X is N or CH;
   R₁ and R₂ are each independently selected from substituted or unsubstituted C₁-C₄ straight-chain or branched alkyl; or R₁ and R₂ combine to form a 5-membered heterocyclic ring;
   R₃ and R₄ are each independently H or C₁-C₄ alkyl;
   each R₅ can be the same or different and is independently selected from H, halogen, C₁-C₄ alkyl, —CF₃, C₁-C₄ alkoxyl, hydroxyl, nitro, amino, alkylamino, dialkylamino, sulfate, cyano, carboxyl, and mercapto;
   each R₆ can be the same or different and is each independently selected from H, halogen, C₁-C₄ alkyl, —CF₃, C₁-C₄ alkoxyl, hydroxyl, nitro, amino, alkylamino, dialkylamino, sulfate, cyano, carboxyl, and mercapto; and
   pharmaceutically acceptable salts thereof.

In certain aspects, the compound of formula (I) is a compound of formula (Ia):

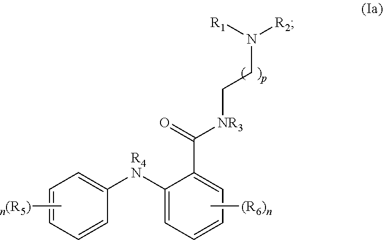

wherein R₁ and R₂ are each independently substituted or unsubstituted C₁-C₄ straight-chain or branched alkyl. In particular aspects, the substituted or unsubstituted C₁-C₄ straight-chain or branched alkyl is selected from methyl, ethyl, n-propyl, isopropyl, n-butyl, isobutyl, sec-butyl, and tert-butyl.

In more certain aspects, the compound of formula (Ia) is:

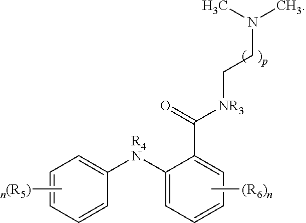

In yet more certain aspects, the compound of formula (I-a) is:

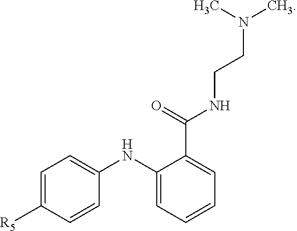

In certain aspects, R₅ is selected from H, halogen, and C₁-C₄ alkoxyl. In particular aspects, R₅ is H. In particular aspects, R₅ is halogen. In particular aspects, R₅ is methoxyl.

In particular aspects, the compound of formula (Ia) is selected from:

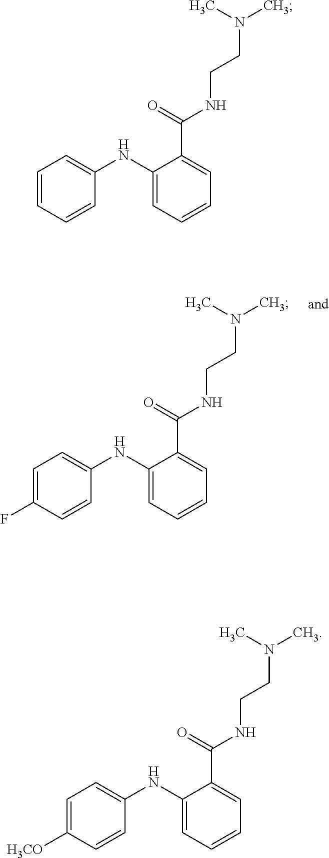 [SMILES: CN(C)CCNC(=O)c1ccccc1Nc1ccccc1 ; CN(C)CCNC(=O)c1ccccc1Nc1ccc(F)cc1 ; COc1ccc(Nc2ccccc2C(=O)NCCN(C)C)cc1]

In certain aspects, the compound of formula (I) is a compound of formula (Ib):

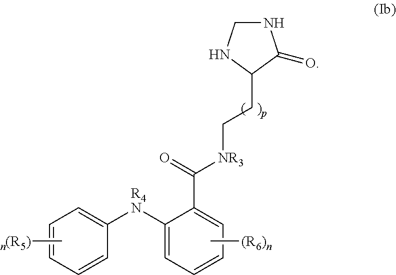

In certain aspects, the compound of formula (Ib) is:

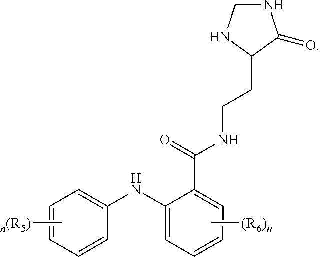

In certain aspects, the compound of formula (Ib) is:

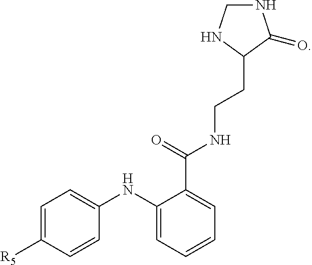

In particular aspects, R₅ is H or halogen. In particular aspects, R₅ is H. In particular aspects, R₅ is halogen.

In particular aspects, the compound of formula (Ib) is selected from:

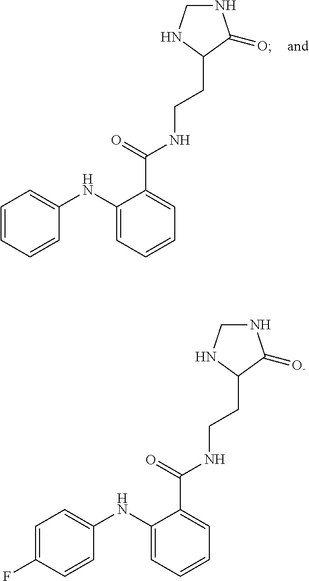 [SMILES: O=C(NCCC1NCNC1=O)c1ccccc1Nc1ccccc1 ; O=C(NCCC1NCNC1=O)c1ccccc1Nc1ccc(F)cc1]

In some aspects, the presently disclosed subject matter provides a composition comprising a compound of formula (I) and a pharmaceutically acceptable carrier.

In some aspects, the presently disclosed subject matter provides a method for treating a disease, condition, or disorder associated with phosphorylation of eukaryotic initiation factor 2 alpha (eIF2α), the method comprising administering to a subject in need of treatment thereof a therapeutically effective amount of a compound of formula (I).

In some aspects, the disease, condition, or disorder associated with phosphorylation of eIF2α comprises a neurodegenerative disease. In certain aspects, the neurodegenerative disease is selected from Alzheimer's disease, Parkinson disease, Creutzfeldt-Jakob disease, Huntington's disease, frontotemporal dementia (FTD), and amyotrophic lateral sclerosis (ALS).

In certain aspects, the administering of a therapeutically effective amount of a compound of formula (I) inhibits microtubule affinity-regulating kinase 2 (MARK2) kinase activity. In particular aspects, inhibiting MARK2 kinase activity reduces phosphorylation of eukaryotic initiation factor 2 alpha (eIF2α). In certain aspects, reducing the phosphorylation of eIF2α reduces the phosphorylation of eIF2α-⁵¹S.

In certain aspects, the phosphorylation of eIF2α is associated with a response to proteotoxic stress. In particular aspects, the proteotoxic stress is associated with protein misfolding. In certain aspects, phosphorylation of eIF2α is associated with regulating translation under stress.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Figures, which are not necessarily drawn to scale, and wherein:

FIG. 1A, FIG. 1B, FIG. 1C, FIG. 1D, FIG. 1E, FIG. 1F, FIG. 1G, and FIG. 1H demonstrate that MARK2 is a direct kinase for eIF2α. (FIG. 1A) In vitro kinase assays using purified proteins and [γ-³²P]-ATP show that MARK2 is a direct kinase for eIF2α. The MARK2-specific Ab, but not the IgG control, abolished the kinase activity. MBP was used as a positive control substrate for the kinase activity of MARK2. The asterisk indicates the autophosphorylated MARK2. (FIG. 1B) Immunoblot analyses of the reaction products from the in vitro kinase assay indicate that MARK 2 phosphorylates eIF2α at its serine 51 residue. PKR was used as a positive control kinase that phosphorylates eIF2α⁵¹S. (FIG. 1C) Left: immunofluorescence of MARK2 (green) and endogenous phosphorylated eIF2α-⁵¹S (red) in MEFs. The arrow points to representative cells at the top with high MARK2 expression, and the arrowhead points to a representative cell at the bottom with low MARK2 expression. Right: quantification of the levels of phosphorylated eIF2α-⁵¹S in cells with high or low levels of MARK2 expression (n=8). (FIG. 1D) The level of phosphorylated eIF2α-⁵¹S is significantly lower in MARK2 knockout MEFs than in WT control cells. Bar graph represents quantification of the immunoblot analysis (n=3). (FIG. 1F) MEFs with elevated expression of MARK2^{WT} showed significantly increased levels of phosphorylated eIF2α-⁵¹S, as compared to MEFs expressing the mutant MARK2^{T595A}. The bar graph represents quantification of the immunoblot analysis (n=4). (FIG. 1F) In vitro kinase assays using [γ-³²P]-ATP and MARK2 variants purified from HEK293 cells show that MARK-2^{WT} is a direct kinase of eIF2α, but the T595A mutation significantly reduced its activity for phosphorylating eIF2α. The bar graph represents quantification of the radiograph analysis (n=3). (FIG. 1G) The NanoBRET donor saturation assay indicates the specificity of the interaction between eIF2α and MARK2, as compared to the positive control PERK interaction with eIF2α and the nonspecific interaction between NanoLuc and HaloTag proteins. (FIG. 1H) The interaction between MARK2 and eIF2α variants, including its WT protein, phosphor-null mutant S51A, and phosphor-mimicking mutant S51D) [n:=6 (Halo+Nano and eIF2α+MARK2), n=3 (p53+MDM2, eIF2α+ERK, eIF2α^{S51A}+MARK2, and eIF2≢^{S51D}+MARK2)]. Scale bar: 10 μm. Error bars represent 1 SEM. *p≤0.05; **p≤0.01; ****p≤0.0001. Ab, antibody; eIF2α, eukaryotic initiation factor 2 alpha; IgG, immunoglobulin G; KO, knockout; MARK2, microtubule affinity-regulating kinase 2; MBP, myelin basic protein; MEF, mouse embryonic fibroblast; PERK, PKR-like ER-resident kinase; PKR, protein kinase R; WT, wild-type;

FIG. 2A, FIG. 21B FIG. 2C, FIG. 21), FIG. 2E, FIG. 2F, and FIG. 2G; illustrate a PKCδ-MARK2-eIF2α signaling cascade in response to protein misfolding stress. (FIG. 2A) Immunoblot analyses of MEFs treated with MG132 indicate that the stress increases the level of phosphorylated MARK2-⁵⁹⁵T and eIF2α-⁵¹S. Bar graphs represent the quantification of the immunoblots (n=3). (FIG. 2B) PERK, PKR, HRI, GCN2, and MARK2 proteins were analyzed by immunoblotting in the 4-KO and 5-KO MEFs as compared to the WT MEFs. (FIG. 2C) Immunoblot analyses of WT, 4-KO (PERK, GCN2, HRI, and PKR), and 5-KO (PERK, PKR, HRI, GCN2, and MARK2) MEFs treated with MG132 indicate that eIF2α-⁵¹S is still phosphorylated in response to the stress in the 4-KO MIFs. The levels of phosphorylated eIF2α-⁵¹S in the 5-KO cells are significantly lower than those in the 4-KO MEFFs. Bar graphs represent the quantification of the immunoblots (n=7; 5-KO without MG132 vs. 5-KO with MG132p=0.1893). (FIG. 2D) PKCδ controls the phosphorylation levels of MARK2-⁵⁹⁵T and eIF2α-⁵¹S, as indicated by immunoblot analyses of PKCδ KO MEFs and the opposite MEFs that stably express elevated levels of PKCδ (GE). Bar graphs represent the quantification of the immunoblots (n=3). (FIG. 2E) In vitro kinase assays demonstrate that PKCδ phosphorylates MARK2. MARK2 exhibits autophosphorylation (lane 2). MBP was used as a positive control substrate. The presence of PKCδ significantly increases the phosphorylation of MARK2 (lane 5) As a candidate substrate, the MARK2 protein level in lane 5 was only half of the MARK2 levels in lanes 2 and 4. The bar graph stows the quantification of the in vitro kinase assays (n=4). (FIG. 2F) Immunoblot analyses of PKCδ KO and control MEFs treated with the proteasome inhibitor, MG132 (500 nM), for 2, 4, or 6 h indicate that the absence of PKCδ abolishes stress-induced phosphorylation of MARK2. Line graphs show the quantification of the immunoblots (n=3). (FIG. 2G) Correlative changes in the protein levels of ATF4 and phosphorylated eIF2α in PKCδ or MARK2 KO and control MEFs, under proteotoxic stress induced by MG132. Bar graph represents quantification of the immunoblots (n=3). Error bars represent±SEM. *p≤0.05; *p≤0.01; ***p≤0.001; ****p≤0.0001. eIF2α, eukaryotic initiation factor 2 alpha; GCN2, general control nonderepressible factor 2 kinase; HRI, heme-regulated eFI2α kinase; KO, knockout; MARK2, microtubule affinity-regulating kinase 2; MBP, myelin basic protein; MEF, mouse embryonic fibroblast; PERK, PKR-like ER-resident kinase; PKCδ, protein kinase C delta; PKR, protein kinase R; WT, wild-type;

FIG. 3A, FIG. 3B, and FIG. 3C demonstrate that HSP90 interacts with PKCδ and mediates proteotoxicity-induced activation of the PKCδ-MARK2-eIf2α signaling pathway. (FIG. 3A) Coimmunoprecipitation analyses of HSP90 as pulled down by the anti-PKCδ antibody in control MEF cells, as compared to those stably expressing PKCδ or completely lacking PKCδ. IgG was used as a control for the anti-PKCδ antibody. The results indicate that PKCδ specifically interacts with HSP90 in a manner dependent on the levels of PKCδ n=3). (FIG. 3B) Coimmunoprecipitation analyses of HSP90 as pulled down by the anti-PKCδ antibody in MEFs cells with or without treatment with MG132 indicate that the proteotoxic stress abolished the interaction between PKCδ and HSP90 (n=3). (FIG. 3C) Immunoblot analyses of MEFs treated with geldanamycin (2 μM, 1 h) versus the DMSO control indicate that inhibition of HSP90 substantially increased die phosphorylation of PKCδ-⁵⁰⁵T, MARK2-⁵⁹⁵T, and eIF2α-⁵¹S. Bar graphs represent the quantification of the immunoblots (n=3). Error bars represent±SEM. *p≤0.05; **p≤0.01; ***p≤0.001. eIF2α, eukaryotic initiation factor 2 alpha; HSP90, heat shock protein 90, IgG, immunoglobulin G; KO, knockout; MARK2, microtubule affinity-regulating kinase 2; MEF, mouse embryonic fibroblast; PKCδ, protein kinase C delta; WT, wild-type;

FIG. 4A, FIG. 4B, FIG. 4C, FIG. 4D, and FIG. 4E demonstrate that the expression of misfolded mutant SOD1 leads to phosphorylation of eIF2α in mammals. (FIG. 4A) The phosphorylation of eIF2α was increased upon expression of SOD1^{G85R} in MEFs as compared to the SOD1^{WT} control. The SOD1 proteins are Flag-tagged, and immunoblot analyses are shown. (FIG. 4B) Immunoblot analyses of spinal cord lysates from NTg (>10 months), SOD1^{WT-γFP} (nonsymptomatic, >10 months), SOD1^{G85R-γFP} (presymptomatic at 7 months and symptomatic at 8 months), and SOD1^{G93A} (symptomatic at 6 months) transgenic mice show an increase in phosphorylation of eIF2α at its serine 51 residue, occurring in a mutant SOD1- and symptom-dependent manner. (FIG. 4C) Quantification of immunoblots in (B) (n=3 independent sets of mice). (FIG. 4D) Immunostaining in the spinal cords from NTg, SOD1^{WT-YFP} (>10 months), and SOD1^{G85R-YFP} (symptomatic at 8 months) mice demonstrates increased phosphorylation of eIF2α-⁵¹S in the symptomatic mutant mice. Scale bar: 25 μm. (FIG. 4E) Immunostaining in the spinal cord from an SOD1^{A4V}-ALS patient and an age-matched human control indicates increased phosphorylation of eIF2α-⁵¹S in the patient's tissue. Scale bar: 100 μm. Error bars represent ±SEM. *p≤0.05. eIF2α, eukaryotic initiation factor 2 alpha; MEF, mouse embryonic fibroblast; NTg, nontransgenic control; Pre, presymptomatic; SOD1, Cu/Zn superoxide dismutase; Symp, symptomatic; WT, wild-type;

FIG. 5A, FIG. 5B, FIG. 5C, FIG. 5D, FIG. 5E, FIG. 5F, FIG. 5G, FIG. 5H, FIG. 5I, FIG. 5J, FIG. 5K, FIG. 5I, and FIG. 5M show that the PKCδ-MARK2-eIF2α signaling pathway is altered in ALS mouse models and patients. (FIG. 5A) Immunoblot analyses of spinal cord lysates from NTg, SOD1^{WT-YFP}, presymptomatic and symptomatic SOD1^{G85R-YFP}, and SOD1^{G93A} transgenic mice show an increase in phosphorylation of PKCδ at its threonine 505 residue, occurring in a mutant SOD1- and symptom-dependent manner. (FIG. 5B, FIG. 5C) Bar graph represents the quantification of the immunoblots in (FIG. 5A) (n=3 sets of mice). (FIG. 5D) Representative immunoblot analyses of PKCδ, MARK2-⁵⁹⁵T, and eIF2α in the spinal cord tissues from ALS patients and non-ALS controls, indicating that increased phosphorylation of PKCδ-⁵⁰⁵T, MARK2-⁵⁹⁵T, and eIF2α-⁵¹S is a general phenotype in patient tissues. (FIG. 5E-FIG. 5G) Bar graph represents the quantification of the immunoblots in (FIG. 5D) (ALS: n=13; CTRL: n=6). (FIG. 5H) Representative immunostaining in the spinal cords from a symptomatic SOD1^{G93}A rouse and an age-matched control shows increased phosphorylation of MARK2-⁵⁹⁵T in the mutant animal. (FIG. 5I) Immunostaining in the spinal cord from an SOD1^{A4V}-ALS patient and an age-matched control indicates increased phosphorylation of PKCδ-⁵⁰⁵T in the patient's tissue. (FIG. 5J) Representative immunostaining in the spinal cord from an SOD1^{A4V}-AL S patient and an age-matched human control indicates increased phosphorylation of MARK2-⁵⁹⁵T in the patient's tissue. (FIG. 5K) Quantification of the number of cells with elevated levels of phosphorylated PKCδ in (FIG. 5H) (n=4 for CTRL and n=4 for ALS patients). (FIG. 5L) Quantification of the number of cells with elevated levels of phosphorylated MARK2 in (FIG. 5I) (n=4 for CTRL and n=4 for SOD1^{G93A}). (FIG. 5M) Quantification of the number of cells with elevated levels of phosphorylated MARK2 in (FIG. 5J) (n=4 for CTRL and n=6 for ALS patients). Scale bars: 50 μm. Error bars represent ±SEM. *p≤0.05; **p≤0.01; ***p≤0.001; ****p≤0.0001. ALS, amyotrophic lateral sclerosis; CTRL, control; eIF2α, eukaryotic initiation factor 2 alpha; MARK2, microtubule affinity-regulating kinase 2; NTg nontransgenic; PKCδ, protein kinase C delta; Pre, presymptomatic; SOD1, Cu/Zn superoxide dismutase; Symp, symptomatic; WT, wild-type;

FIG. 6A, FIG. 6B, FIG. 6C, FIG. 6D, FIG. 6F, FIG. 6F, FIG. 6G, FIG. 6H, and FIG. 6I demonstrate that MARK2 is a specific and direct kinase for eIF2α. (FIG. 6A) Coomassie blue gel staining confirms the high purity of the proteins used in the in vitro kinase activity assays. (FIG. 6B) In vitro kinase assays using purified proteins and [γ-³²P]-ATP demonstrate that PKCδ is not a direct kinase for eIF2α. MBP was used as a positive control substrate for the kinase activity of PKCδ. PKR was used as a positive control for eIF2α kinase activity (lane 5). (FIG. 6C-FIG. 6E) Kinetic analysis of the reactions between the kinase, PKR, MJAR2^{WT}, or MARK2^{KD} (kinase-dead mutant), and the substrate MBP using the Kinase-Glo assay quantifying ATP consumption via luminescent signals. Initial velocities represented by ATPs incorporated into the substrate were plotted against the kinase to determine the Km and Vmax of PKR, MARK2^{WT}, and MARK2^{KD}. (FIG. 6F-FIG. 6H) Kinetic analysis of the reactions between the kinases and the substrates eIF2α^{WT} and eIF2α^{S51A} using the Kinase-Glo assay. (FIG. 6I) In vitro kinase assays based on radiolabeling and gel electrophoresis using proteins purified from E. coli demonstrate that MARK2 directly phosphorylates eIF2α at serine 51. The kinase-dead MARK2^{KD} mutant did not show activity toward eIF2α^{WT} or eIFα^{S51A}. eIF2α, eukaryotic initiation factor 2 alpha; MARK2, microtubule affinity-regulating kinase 2; MBP, myelin basic protein: PKCδ, protein kinase C delta; PKR, protein kinase R; WT, wild-type;

FIG. 7A, FIG. 7B, FIG. 7C, FIG. 7D, FIG. 7E, FIG. 7F, and FIG. 7G are schematics of CRISPR editing and NanoBRET analysis. The CRISPR/Cas9-induced null mutations were generated to create knockout cells lacking single or multiple eIF2C kinases. The 4-KO MEFs were generated by deleting PERK, HRI, and GCN2 from an existing PRK knockout MEF line. The 5-KO MEFs were generated by deleting MARK2 from the 4-KO MEFs lacking PERK, PRK, HRI, and GCN2. The 4-KO′ and 5-KO′ MEFs were generated by introducing deletion mutations in exon 5 of the PKR gene, resulting in the removal of a remnant C-terminal fragment of PKR from the existing 4-KO and 5-KG MEF lines. In addition to Sanger sequencing to confirm the DNA mutation, the deletion of PERK, PKR, HRI, GCN2, and MARK2 was verified by immunoblotting. (FIG. 7A) In the MARK2 knockout HAP1 cell line, the human MARK2 gene is disrupted with a CRISPR/Cas9-induced 11-bp deletion (GATTCGGGGCC) in exon 2, resulting in a premature stop codon (TGA) in exon 2 and disruption of the MARK2 gene in the near-haploid genome. (FIG. 7B) In the 5-KO MEF line, the MARK2 gene is disrupted with a CRISPR/Cas9-induced 1-bp insertion in exon 2, resulting in a premature stop codon (TGA) in exon 2 in both alleles of the gene (FIG. 7C) In the 4-KO and 5-KO MEF lines, the PERK gene is disrupted with a CRISPR/Cas9-induced ½-bp deletion in exon 1, resulting in a premature stop codon (TGA or TAA) in exon 2 in both alleles of the gene. (FIG. 7D) In the 4-KO and 5-KO MEF lines, the HRI gene is disrupted with a CRISPR/Cas9-induced 1-bp or 4-bp deletion in exon 1, resulting in a premature stop codon (TA A) in exon 2 in both alleles of the gene. (FIG. 7E) In the 4-KO and 5-KO MEF lines, the GCN2 gene is disrupted with a CRISPR/Cas9-induced 13-bp or 6-bp deletion in exon 2, resulting in a premature stop codon (TGA or TAA) in exon 2 in both alleles of the gene. (FIG. 7F) In the 4-KO′ and 5-KG′ MEF lines, the existing PKR knockout allele (4-KO and 5-KO) is further edited using CRISPR to disrupt a remnant C-terminal fragment of PKR. In the original PKR knockout allele, exons 2 and 3 were replaced with a segment containing the NEO-UMS cassette, which functions as a translational stop. Due to exon skipping, a C-terminal fragment of PKR containing dsRBM2 and the kinase domain could still be generated as depicted. To remove the C-terminal fragment, we used CRISPR to introduce a 1-bp or 11-bp deletion in exon 5 at the 2 alleles, resulting in premature stop codons (TAA or TAG) in exon 5 or 6, respectively, of the 2 alleles. (FIG. 7G) Workflow for the NanoBRET assay to monitor the interaction between MARK2 and eIF2α in live HEK293 cells. eIF2α, eukaryotic initiation factor 2 alpha; GCN2, general control nonderepressible factor 2 kinase; FRI, heme-regulated eIF2α kinase; MARK2, microtubule affinity-regulating kinase 2; MEF, mouse embryonic fibroblast; NEO-UMS, neomycin and upstream mouse sequence, PERK, PKR-like ER-resident kinase; PKR, protein kinase R;

FIG. 8A, FIG. 8B, FIG. 8C, FIG. 8D, FIG. 8E, and FIG. 8F demonstrate that the activation of MARK2 is correlated with the phosphorylation of eIF2α, and the signaling pathway is independent of the previously known kinases. (FIG. 8A) Deletion of the MARK2 gene decreases the phosphorylation of eIF2α-⁵¹S in human HAP1 cells. Bar graph represents quantification of the immunoblots (n=3). (FIG. 8B) Immunoblotting analyses of MEFs treated with the proteasome inhibitor MG132 (500 nM) indicate increased levels of phosphorylated eIF2α-⁵¹S that correlated with the levels of phosphorylated MARK2-⁵⁹⁵T over the 24-h time course of the MG132 treatment. The graph used to calculate the Pearson coefficient is shown to indicate the significant correlation (p=0.0161). (FIG. 8C-FIG. 8F) Immunoblotting analyses of WT and knockout MEFs treated with MG132 indicate that the PKCδ-MARK2-eIF2α signaling pathway can be activated, as measured by the levels of phosphorylated eIF2α-⁵¹S, MARK2-⁵⁹⁵T, and PKCδ-⁵⁰⁵T independently of any of the previously known eIF2α kinases, including PERK, HRI, PKR, and GCN2. Bar graphs represent the quantification of the immunoblots (n=3). Error bars represent ±SEM. * p≤0.05: **p≤0.01. eIF2α, eukaryotic initiation factor 2 alpha; GCN2, general control nonderepressible factor 2 kinase; HRI, heme-regulated eIF2α kinase; KO, knockout; MARK2, microtubule affinity-regulating kinase 2; MEF, mouse embryonic fibroblast; PERK, PKR-like ER-resident kinase; PKCδ, protein kinase C delta; PKR, protein kinase R; WT, wild-type;

FIG. 9A, FIG. 9B, FIG. 9C, FIG. 9D, FIG. 9E, FIG. 9F, FIG. 9G, and FIG. 9H illustrate MARK2-mediated eIF2α-⁵¹S phosphorylation, translational attenuation, and the characterization of MEFs lacking multiple eIF2α kinases. (FIG. 9A) The specificity of the antibody against phosphorylated eIF2α-⁵¹S was verified in an eIF2α^{S51A} knock-in mutant MEF line, in which the S51A mutation abolished the immunoblot signal of phosphorylated eIF2α-⁵¹S observed in WT MEFs treated with MG132. (FIG. 9B) MEFs stably overexpressing MARK2^{WT} or MARK2^{T595A} were pulsed-labeled with puromycin for 10 min, and the cell lysates were analyzed by SDS-PAGE and immunoblotting against puromycin-labeled proteins. The bar graph represents the quantification of the immunoblots (n=3). (FIG. 9C) MEFs stably overexpressing MARK2^{WT} or MARK2^{T595A} were pulse-labeled with ³⁵S-methionine and ³⁵S-cysteine for 1 h, and the cell lysates were analyzed by liquid scintillation counting for ³⁵S-labeled proteins (n=3). The quantitative results indicate that overexpression of MARK2^{WT} caused attenuation of global translation, while the T595A mutation impaired its regulatory activity. (FIG. 9D)) A time course of the treatment with MG132 (20 μM) at indicated times shows that the phosphorylation of eIF2α-⁵¹S peaked around 4 h in the MEFs. (FIG. 9E) Immunoblot analyses of MEFs treated with sodium arsenite (200 μM, 1 h) indicate that the phosphorylation of MARK2-⁵⁹⁵T and eIF2α-⁵¹S was increased by the stress in WT and 4-KO cells. Bar graphs represent the quantification of the immunoblots (n=3). (FIG. 9F) Immunoblot analyses of MEFs treated with tunicamycin (24 μg/ml, 2 h) indicate that the phosphorylation of MARK2-⁵⁹⁵T was not affected by the stress, while the phosphorylation of eIF2α-⁵¹S could be independently induced in WT but not the 4-KO cells. Bar graphs represent the quantification of the immunoblots (n=3). (FIG. 9G) Immunoblot analyses of WT, 5-KO, 4-KO′, and 5-KO′ MEFs treated with mINF-α (1,000 U/mL for 18 h) indicate a remnant C-terminal fragment of PKR in 5-KO cells, which has been deleted in 4-KO′ and 5-KG′ cells as designed, (FIG. 9H) Immunoblot analyses of WT, 4-KO′, and 5-KO′ MEFs treated with MG132 indicate that eIF2α-⁵¹S is phosphorylated in response to the stress in the 4-KO′ MEFs. The levels of phosphorylated eIF2α-⁵¹S in the 5-KO′ cells are significantly lower than those in the 4-KO′ MEFs (n=3). The levels of phosphorylated PP1α-³²⁰T in the 4-KG′ and 5-KG′ cells are significantly lower than those in WT MEFs, and there is a trend for an increase in the phosphorylation of PP1α-³²⁰T in the 5-KO′ MEFs compared to the 4-KG′ MEFs (n=3; 4-KO′ with MG132 vs. 5-KO′ with MG132, Student t test p=0.0191, nonsignificant using one-way ANOVA with the Tukey post hoc test). Error bars represent±SEM. *p≤0.05; **p≤0.01; ***p≤0.001; ****p≤0.0001 eIF2α, eukaryotic initiation factor 2 alpha; KO, knockout; MARK2, microtubule affinity-regulating kinase 2; MEF, mouse embryonic fibroblast; mINF-α, mouse interferon-α; PKR, protein kinase R; WT, wild-type;

FIG. 10A and FIG. 10B show that PKCδ interacts with HSP90 but not HSP70 and mediates proteotoxicity-induced activation of PKCδ-MARK2-eIF2α signaling. (FIG. 10A) In coimmunoprecipitation analyses, no HSP70 was detected in immunoprecipitates pulled down by the anti-PKCδ antibody from WT MEFs, those from PKCδ KO MEFs, or those from MEFs stably expressing PKCδ. IgG was used as a control for the anti-PKCδ antibody. (FIG. 10B) Comparison of WT MEFs and those with HSP90 knockdown by CRISPR in immunoblot analyses indicate that the down-regulation of HSP90 substantially increased the phosphorylation of PKCδ-⁵⁰⁵T, MARK2-⁵⁹⁵T, and eIF2α-⁵¹S. eIF2α, eukaryotic initiation factor 2 alpha; HSP70, heat shock protein 70; HSP90, heat shock protein 90; IgG, immunoglobulin G; IB, immunoblotting; IP, immunoprecipitation; KO, knockout; MARK2, microtubule affinity-regulating kinase 2; MEF, mouse embryonic fibroblast; PKCδ, protein kinase C delta; WT, wild-type;

FIG. 11A, FIG. 11B, FIG. 11C, FIG. 11D, and FIG. 11E demonstrate that increased phosphorylation of MARK2 occurs in the affected tissues of a mutant SOD1-induced ALS mouse model. (FIG. 11A) Immunoblot analyses of spinal cord lysates from NTg, SOD1^{WT-YFP}, presymptomatic and symptomatic SOD1^{G85R-YFP}, and SO1^{G93A} transgenic mice show no change in the levels of phosphorylation of PKCδ at tyrosine 311. (FIG. 11B) Immunoblot analyses of spinal cords from symptomatic SOD1^{G93A} nice and NTg littermate controls indicate that the level of phosphorylated PERK-⁹⁸⁰T was significantly increased in the SOD1^{G93A} nice, while no change was detected for GCN2 Immunohistochemical analyses of phosphorylated MARK2 in the brain cortex (FIG. 11C), midbrain (FIG. 11D), and striatum (FIG. 11E) from symptomatic SOD1^{G93A} transgenic mice and NTg controls. The staining of phosphorylated MARK2-⁵⁹⁵T in all 3 brain regions is increased in the So1^{G93A} mice as compared to NTg mice. Error bars represent ±SEM. *p≤0.05. Scale bar: 50 μm. ALS, amyotrophic lateral sclerosis; GCN2, general control nonderepressible factor 2 kinase; MARK2, microtubule affinity-regulating kinase 2; n.s., nonsignificant; NTg, nontransgenic; PERK, PKR-like ER-resident kinase; PKCδ, protein kinase C delta; Pre, presymptomatic; SOD1, Cu/Zn superoxide dismutase; Symp, symptomatic; WT, wild-type;

FIG. 12A, FIG. 12B, and FIG. 12C demonstrate that ALS patients' tissues exhibit increased phosphorylation of MARK2. (FIG. 12A) Representative immunoblot analyses of PKCδ, MARK2-⁵⁹⁵T, and eIF2α in the spinal cord tissues from ALS patients and non-ALS controls, indicating that increased phosphorylation of PKCδ-⁵⁰⁵T, MARK2-⁵⁹⁵T, and eIF2α-⁵¹S is a general phenotype in patient tissues. (FIG. 12B) Immunohistochemical staining of phosphorylated MARK2-⁵⁹⁵T in the spinal cords from an SOD1^{A4V}-ALS patient, an sALS patient, and a non-ALS age-matched control case. (FIG. 12C) Immunostaining for phosphorylated MARK2-⁵⁹⁵T in the motor cortex of 3 different sporadic ALS patients. Increased phosphorylation of MARK2-⁵⁹⁵T is observed in all ALS patient tissues. Scale bar: 50 pin. ALS, amyotrophic lateral sclerosis; CTRL, control; eIF2α, eukaryotic initiation factor 2 alpha; fALS, familial ALS; MARK2, microtubule affinity-regulating kinase 2; PKCδ, protein kinase C delta; sALS, sporadic ALS; SOD1, Cu/Zn superoxide dismutase;

FIG. 13 represents a model for the PKCδ-MARK2-eIF2α signaling pathway. Upon protein misfolding stress, HSP90 is sequestered by misfolded proteins, resulting in phosphorylation and activation of PKCδ, which in turn activates MARK2 that phosphorylates eIF2α. The increased phosphorylation of eIF2α leads to translational attenuation. eIF2α, eukaryotic initiation factor 2 alpha; HSP90, heat shock protein 90; MARK2, microtubule affinity-regulating kinase 2; PKCδ, protein kinase C delta;

FIG. 14A, FIG. 14B, FIG. 14C, FIG. 14D, FIG. 14E, FIG. 14F, FIG. 14G, FIG. 14H, and FIG. 14I demonstrate that MARK2 is a specific and direct kinase for eIF2α. (FIG. 14A) In vitro kinase assays using purified proteins and [γ-³²P]-ATP demonstrate that MARK2, but not PKCδ, is a direct kinase for eIF2α. Myelin basic protein (MBP) was used as a positive control substrate for the kinase activity of PKCδ. PKR was used as a positive control for eIF2α kinase activity (lane 13). (FIG. 14B) Immunoblot analyses of the reaction products from the in vitro kinase assay indicate that MARK 2 phosphorylates eIF2α at its ⁵¹S residue. PKR was used as a positive control kinase that phosphorylates eIF2α-⁵¹S. (FIG. 14C) In vitro kinase assays based on radiolabeling and gel electrophoresis using proteins purified from E. coli demonstrate that MARK2 directly phosphorylates eIF2α at serine 51. The kinase-dead MARK2^{KD} mutant did not show activity towards eIF2α^{WT} or eIF2^{S51A}. (FIG. 14D, FIG. 14E, FIG. 14F) Kinetic analysis of the reactions between the kinase, PKR, MARK2^{WT}, or MARK2^{KD} (kinase-dead mutant), and the substrate MBP using the Kinase-Glo assay quantifying ATP consumption via luminescent signals. Initial velocities represented by ATPs incorporated into the substrate were plotted against the kinase to determine the Km and Vmax of PKR, MARK2^{WT}, and MARK2^{KD}. (FIG. 14G, FIG. 14H, FIG. 14I) Kinetic analysis of the reactions between the kinases and the substrates eIF2α^{WT} and eIF2α^{S51A} using the Kinase-Glo assay;

FIG. 15A, FIG. 15B, FIG. 15C, FIG. 15D, FIG. 15E, FIG. 15F, FIG. 15G, and FIG. 15H demonstrate that MARK2 is a direct kinase for eIF2α in mammalian cells. (FIG. 15A) Left: Immunofluorescence of MARK2 (green) and endogenous phosphorylated eIF2α-⁵¹S (red) in MEFs. The arrow points to two cells at the top with high MARK2 expression, and the arrowhead points to one cell at the bottom with low MARK2 expression. Right: Quantification of the levels of phosphorylated eIF2α-⁵¹S in cells with high or low levels of MARK2 expression (n=8). (FIG. 15B) Knockout of the MARK2 gene decreases the phosphorylation of eIF2α-⁵¹S in human HAP1 and MEFs cells. Bar graph represents quantification of the immunoblots (n=3). (FIG. 15C) MEFs with elevated expression of WT MARK2 showed significantly increased levels of phosphorylated eIF2α-⁵¹S, as compared to MEFs expressing the mutant MARK2T⁹⁵A. The bar graph represents quantification of the immunoblot analysis (n=4). (FIG. 15D) The specificity of the antibody against phosphorylated eIF2α-⁵¹S was verified in an eIF2α^{S51A} knock-in mutant MEF line, in which the S51A mutation abolished the immunoblot signal of phosphorylated eIF2α-⁵¹S observed in WT MEFs treated with MG132. (FIG. 15E) WT and MARK2 overexpression MEFs were pulse-labeled with ³⁵S-methionine and ¹⁵S-cysteine for 1 hr, and the cell lysates were analyzed by SDS-PAGE autoradiography (left) and liquid scintillation counting (right). Results indicate that overexpression of MARK2 caused attenuation of global translation. (FIG. 15F) In vitro kinase assays using [γ-³²P]-ATP and MARK2 variants purified from IHEK293 cells show that WT MARK2 is a direct kinase of eIF2α, but the T595A mutation significantly reduced its activity for phosphorylating eIF2α. The bar graph represents quantification of the radiograph analysis (n=3). (FIG. 15G) The NanoBRET donor saturation assay indicates the specificity of the interaction between eIF2α and MARK2, as compared to the positive control PERK interaction with eIF2α and the non-specific interaction between NanoLuc and HaloTag proteins. (FIG. 15H) The interaction between MARK2 and eIF2α variants, including its wild-type protein, phosphor-null mutant S51A, and phosphor-mimicking mutant S51D [n=6 (Halo+Nano and eIF2α+MARK2), n=3 (p53+MDM2, eIF2α+PERK, eIF2α^{S51A}+MARK2 and eIF2α^{S51D}+MARK2)]. Scale bar: 10 μm. Error bars represent±SEM. *p≤0.05; **p≤0.01; ****p≤0.0001;

FIG. 16A, FIG. 16B, FIG. 16C, FIG. 16D, FIG. 16E, and FIG. 16F demonstrate that the activation of MARK2 is correlated with the phosphorylation of eIF2α kinases under stress. (FIG. 16A) Immunoblot analyses of MEFs treated with MG132 indicate that the stress increases the level of phosphorylated MARK2-595T and eIF2α-⁵¹S. Bar graphs represent the quantification of the immunoblots (n=3). (FIG. 16B) Immunoblotting analyses of MEFs treated with the proteasome inhibitor MG132 (500 nM) indicate increased levels of phosphorylated eIF2α-⁵¹S that correlated with the levels of phosphorylated MARK2-⁵⁹⁵T over the 24-hr time course of the MG132 treatment. The graph used to calculate the Pearson coefficient is shown to indicate the significant correlation (p=0.0161). (FIG. 16C, FIG. 16D, FIG. 16E, FIG. 16F) Immunoblotting analyses of WT and knockout MEFs treated with MG132 indicate that the PKCδ-MARK2-eIF2α signaling pathway can be activated, as measured by the levels of phosphorylated eIF2α-⁵¹S, MARK2-⁵⁹⁵T, and PKCδ-⁵⁰⁵T independently of any of the previously known eIF2α kinases, including PERK, HRI, PKR, and GCN2. Bar graphs represent the quantification of the immunoblots (n=3). Error bars represent±SEM. *p≤0.05; **p≤0.01;

FIG. 17A, FIG. 17B, FIG. 17C, FIG. 17D, FIG. 17E, FIG. 17F, and FIG. 17G demonstrate that MARK2 mediates phosphorylation of eIF2α under stress. (FIG. 17A) Immunoblot analyses of MEFs treated with MG132 indicate that the stress increases the level of phosphorylated MARK2-⁵⁹⁵T and eIF2α-⁵¹S. Bar graphs represent the quantification of the immunoblots (n=3). (FIG. 17B) PERK, PKR, HRI, GCN2 and MARK2 proteins were analyzed by immunoblotting in the 4-KO and 5-KO MEFs as compared to the WT MEFs. of the immunoblots (n=3). (FIG. 17B) PERK, PKR, HRI, GCN2 and MARK2 proteins were analyzed by immunoblotting in the 4-KO and 5-KO MEFs as compared to the WT MEFs. (FIG. 17C) A time course of the treatment with MG132 (20 μM) at indicated times shows that the phosphorylation of eIF2α-⁵¹S peaked around 4 hr in the MEFs. (FIG. 17D) Immunoblot analyses of MEFs treated with sodium arsenite (200 M, 1 hr) indicate that the phosphorylation of MARK2-⁵⁹⁵T and eIF2α-⁵¹S was increased by the stress in WT and 4-KO cells. Bar graphs represent the quantification of the immunoblots (n=3). (FIG. 17E) Immunoblot analyses of MEFs treated with tunicamycin (24 μg/mL, 2 hr) indicate that the phosphorylation of MARK2-⁵⁹⁵T was not affected by the stress while the phosphorylation of eIF2α-⁵¹S could be independently induced in WT but not the 4-KO cells. Bar graphs represent the quantification of the immunoblots (n=3). (FIG. 17F) Immunoblot analyses of WT, 5-KO, 4-KO′, and 5-KO′ MEFs treated with mINF-α (mouse interferon-α, 1000 U/mL for 18 hr) indicate a remnant C-terminal fragment of PKR in 5-KO cells, which has been deleted in 4-KO′ and 5-KO′ cells as designed. (FIG. 17G) Immunoblot analyses of WT, 4-KO′, and 5-KO′ MEFs treated with MG132 indicate that eIF2α-⁵¹S is phosphorylated in response to the stress in the 4-KO′ MEFs. The levels of phosphorylated eIF2α-⁵¹S in the 5-KO′ cells are significantly lower than those in the 4-KO′ MEFs (n=3). The levels of phosphorylated PP1α-³²⁰T in the 4-KO′ and 5-KO′ cells are significantly lower than those in WT MEFs, and there is a trend for an increase in the phosphorylation of PP1α-³²⁰T in the 5-KO′ MEFs compared to the 4-KO′ MEFs (n=3; 4-KO′ with MG132 vs. 5-KO′ with MG132, Student's t-test p=0.0191, non-significant using one-way ANOVA with the Tukey post hoc test). Error bars represent ±SEM. *p≤0.05; **p≤0.01; ***p≤0.001; ****p≤0.0001;

FIG. 18A, FIG. 18B, and FIG. 18C demonstrate that PKCδ-MARK2-eIF2α signaling cascade in response to protein misfolding stress. (FIG. 18A) PKCδ controls the phosphorylation levels of MARK2-⁵⁹⁵T and eIF2α-⁵¹S, as indicated by immunoblot analyses of PKCδ knockout (KO) MEFs and the opposite MEFs that stably express elevated levels of PKCδ (OE). Bar graphs represent the quantification of the immunoblots (n=3). (FIG. 18B) In vitro kinase assays demonstrate that PKCδ phosphorylates MARK2. MARK2 exhibits autophosphorylation (lane 2). MBP was used as a positive control substrate. The presence of PKCδ significantly increases the phosphorylation of MARK2 (lane 5). As a candidate substrate, the MARK2 protein level in lane 5 was only half of the MARK2 levels in lanes 2 and 4. The bar graph shows the quantification of the in vitro kinase assays (n=4). (FIG. 18C) Correlative changes in the protein levels of ATF4 and phosphorylated eIF2α in PKCδ or MARK2 MEFs and control cells, under proteotoxic stress induced by MG132. Bar graph represents quantification of the immunoblots (n=3). Error bars represent±SEM. *p≤0.05; **p≤0.01; ***p≤0.001; ****p≤0.0001;

FIG. 19A, FIG. 19B, and FIG. 19C demonstrate that HSP90 interacts with PKCδ and mediates proteotoxicity-induced activation of the PKCδ-MARK2-eIF2α signaling pathway. (FIG. 19A) Coimmunoprecipitation analyses of HSP90 as pulled down by the anti-PKCδ antibody in control MEF cells, as compared to those stably expressing PKCδ or completely lacking PKCδ. IgG was used as a control for the anti-PKCδ antibody. The results indicate that PKCδ specifically interacts with HSP90 in a dose-dependent manner (n=3). (FIG. 19B) Coimmunoprecipitation analyses of HSP90 as pulled down by the anti-PKCδ antibody in MEFs cells with or without treatment with MG132 indicate that the proteotoxic stress abolished the interaction between PKCδ and HSP90 (n=3). (FIG. 19C) Immunoblot analyses of MEFs treated with geldanamycin (2 M, 1 hr) versus the DMSO control indicate that inhibition of HSP90 substantially increased the phosphorylation of PKCδ-⁵⁰⁵T, MARK2-⁵⁹⁵T, and eIF2α-⁵¹S. Bar graphs represent the quantification of the immunoblots (n=3). Error bars represent±SEM. *p≤0.05; **p≤0.01; ***p≤0.001;

FIG. 20A, FIG. 20B, FIG. 20C, FIG. 20D, FIG. 20E, FIG. 20F, FIG. 20G, FIG. 20H, and FIG. 20I demonstrate that the PKCδ-MARK2-eIF2α signaling pathway is altered in ALS mouse models and patients. (FIG. 20A) Representative immunoblot analyses of PKCδ, MARK2-⁵⁹⁵T and eIF2α in the spinal cord tissues from ALS patients and healthy controls, indicating that increased phosphorylation of PKCδ-⁵⁰⁵T, MARK2-⁵⁹⁵T and eIF2α-⁵¹S is a general phenotype in patient tissues. (FIG. 20B, FIG. 20C, FIG. 20D) Bar graph represents the quantification of the immunoblots in (FIG. 20G) (ALS: n=13; CTRL: n=6). (FIG. 20E) Immunostaining in the spinal cord from an SOD1^{A4V}-ALS patient and an age-matched control indicates increased phosphorylation of PKCδ-⁵⁰⁵T and MARK2-⁵⁹⁵T in the patient's tissue. (FIG. 20F) Representative immunostaining in the spinal cords from a symptomatic SOD1^{G93A} mouse and an age-matched control shows increased phosphorylation of MARK2-⁵⁹⁵T in the mutant animal. (FIG. 20G) Quantification of the number of cells with elevated levels of phosphorylated PKCδ in (FIG. 20E) (n=4 for CTRL and n=4 for ALS patients). (FIG. 20H) Quantification of the number of cells with elevated levels of phosphorylated MARK2 in (FIG. 20F) (n=4 for CTRL and n=4 for SOD1^{G93A}). (FIG. 20I) Quantification of the number of cells with elevated levels of phosphorylated MARK2 in (FIG. 20E) (n=4 for CTRL and n=6 for ALS patients). Scale bars: 50 μm. Error bars represent±SEM. *p≤0.05; **p≤0.01; ***p≤0.001; and

FIG. 21A, FIG. 21B, FIG. 21C, FIG. 21D, FIG. 21E, FIG. 21F, FIG. 21G, and FIG. 21H demonstrate that the newly designed specific inhibitors inhibit the MARK2 kinase activity and reduce the phosphorylation of eIF2α. (FIG. 21A) Immunoblot analyses of MEFs treated with SB216763 and SB415286 (20 μM, 1 hr) versus the DMSO control indicate that inhibition of MARK2 substantially reduces the phosphorylation of eIF2α-⁵¹S. Bar graphs represent the quantification of the immunoblots (n=3). (FIG. 21B) Immunoblot analyses of MEFs treated with Gefitinib (40 μM, 1 hr) and CHIR99021 (20 μM, 1 hr) versus the DMSO control indicate that CHIR99021 decreased the phosphorylation level of eIF2α-⁵¹S but Gefitinib needs a higher concentration than CHIR99021 to achieve the similar effect. Bar graphs represent the quantification of the immunoblots. (FIG. 21C) The structural simulation for the interactions of seven different compounds with MARK2 protein. (FIG. 21D) The structural simulation analysis for SB216763, Gefinitib, and SB415286 show that these drugs have a similar interaction site on MARK2, which is close to the end of the kinase domain and the N-terminus of the UBA domain. (FIG. 21E) The correlation analysis of inhibition of eIF2α phosphorylation with the MARK2-compound affinity indicates that SB216763 and SB415286 have high MARK2 affinity and high activity for inhibiting eIF2α phosphorylation. (FIG. 21F) Structures of the newly designed drugs include YA8075, NH1010, NH1023, YA8076, and NH1018. (FIG. 21G) Western blot analyses of MEFs treated with YA8075 (10 μM, 1 hr) and YA8076 (5 μM, 1 hr) in the presence of MG132 (20 μM, 1 hr) indicate that both YA8075 and YA8076 substantially decrease the phosphorylation of eIF2α-⁵¹S, and YA8076 has a stronger activity in inhibiting the phosphorylation of eIF2α-⁵¹S. Bar graphs represent the quantification of the immunoblots (n=3). (FIG. 21H) Western blot analyses of MEFs treated with Gefitinib (10 μM, 1 hr), CHIR99021(10 μM, 1 hr), YA8075 (10 μM, 1 hr), YA8076 (10 μM, 1 hr), NH1018 (10 μM, 1 hr), or NNH1023(10 μM, 1 hr) in the presence of MG132 (20 μM, 1 hr), indicating that YA8075, YA8076, and NH1018 can decrease the phosphorylation of eIF2α-⁵¹S and YA8076 has the strongest activity in inhibiting the phosphorylation of eIF2α-⁵¹S. Bar graphs represent the quantification of the immunoblots (n=3).

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed. many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

The presently disclosed subject matter provides the identification of a direct kinase of eIF2α, microtubule affinity-regulating kinase 2 (MARK2), which phosphorylates eIF2α in response to proteotoxic stress. The activity of MARK2 was confirmed in the cells lacking the four previously known eIF2α kinases. MARK2 itself was found to be a substrate of protein kinase C delta (PKCδ), which serves as a sensor for protein misfolding stress through a dynamic interaction with heat shock protein 90 (HSP90). Both MARK2 and PKCδ are activated via phosphorylation in proteotoxicity-associated neurodegenerative mouse models and in human patients with amyotrophic lateral sclerosis (ALS). These results reveal a PKCδ-MARK2-eIF2α cascade that may play a critical role in cellular proteotoxic stress responses and human diseases. Inhibitors of MARK2 also are disclosed.

### A. Compounds of Formula (1)

In some embodiments, the presently disclosed subject matter provides a compound of formula (I):

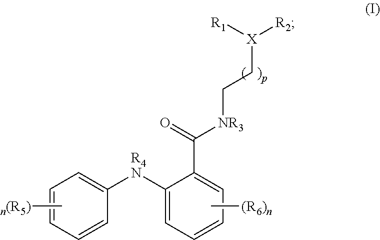

wherein:
   m is an integer selected from 0, 1, 2, 3, 4, and 5;
   n is an integer selected from 0, 1, 2, 3, and 4;
   p is an integer selected from 0, 1, 2, 3, 4, 5, 6, and 7;
   X is N or CH;
   R₁ and R₂ are each independently selected from substituted or unsubstituted C₁-C₄ straight-chain or branched alkyl; or R₁ and R₂ combine to form a 5-membered heterocyclic ring;
   R₃ and R₄ are each independently H or C₁-C₄ alkyl;
   each R₅ can be the same or different and is independently selected from H, halogen, C₁-C₄ alkyl, —CF₃, C₁-C₄ alkoxyl, hydroxyl, nitro, amino, alkylamino, dialkylamino, sulfate, cyano, carboxyl, and mercapto;
   each R₆ can be the same or different and is each independently selected from H, halogen, C₁-C₄ alkyl, —CF₃, C₁-C₄ alkoxyl, hydroxyl, nitro, amino, alkylamino, dialkylamino, sulfate, cyano, carboxyl, and mercapto; and
   pharmaceutically acceptable salts thereof.

In certain embodiments, the compound of formula (I) is a compound of formula (Ia):

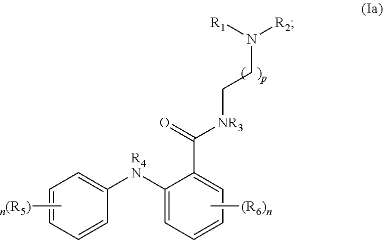

wherein R₁ and R₂ are each independently substituted or unsubstituted C₁-C₄ straight-chain or branched alkyl. In particular embodiments, the substituted or unsubstituted C₁-C₄ straight-chain or branched alkyl is selected from methyl, ethyl, n-propyl, isopropyl, n-butyl, isobutyl, sec-butyl, and tert-butyl.

In more certain embodiments, the compound of formula (Ia) is:

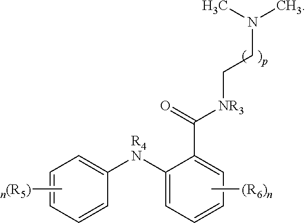

In yet more certain embodiments, the compound of formula (I-a) is:

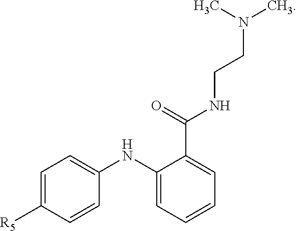

In certain embodiments, R₅ is selected from H, halogen, and C₁-C₄ alkoxyl. In particular embodiments, R₅ is H. In particular embodiments, R₅ is halogen. In particular embodiments, R₅ is methoxyl.

In particular embodiments, the compound of formula (Ia) is selected from:

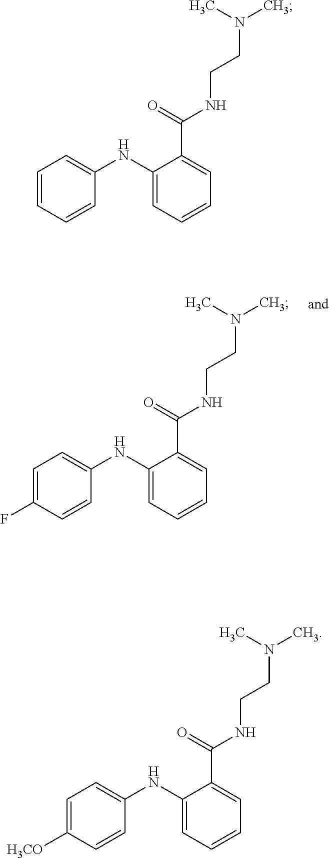 [SMILES: CN(C)CCNC(=O)c1ccccc1Nc1ccccc1 ; CN(C)CCNC(=O)c1ccccc1Nc1ccc(F)cc1 ; COc1ccc(Nc2ccccc2C(=O)NCCN(C)C)cc1]

In certain embodiments, the compound of formula (I) is a compound of formula (Ib):

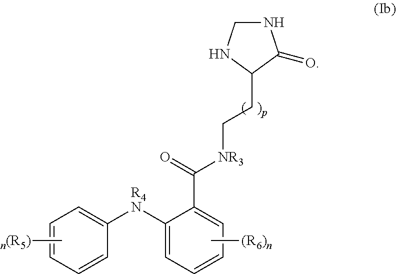

In certain embodiments, the compound of formula (Ib) is:

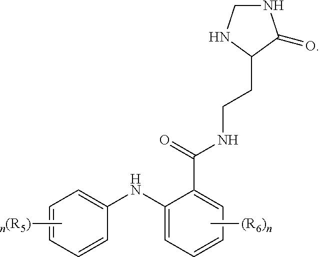

In certain embodiments, the compound of formula (Ib) is:

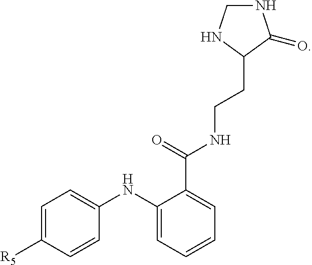

In particular embodiments, R₅ is H or halogen. In particular embodiments, R₅ is H. In particular embodiments, R₅ is halogen.

In particular embodiments, the compound of formula (Ib) is selected from:

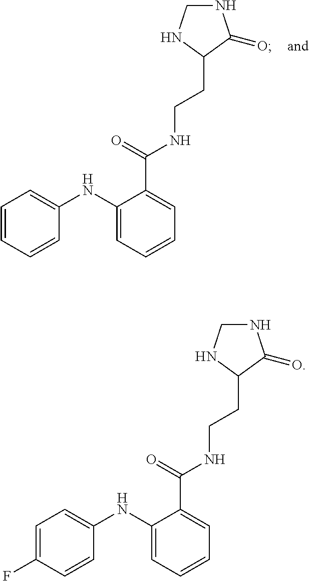 [SMILES: O=C(NCCC1NCNC1=O)c1ccccc1Nc1ccccc1 ; O=C(NCCC1NCNC1=O)c1ccccc1Nc1ccc(F)cc1]

In some embodiments, the presently disclosed subject matter provides a composition comprising a compound of formula (I) and a pharmaceutically acceptable carrier.

B. Method for Treating a Disease, Condition, or Disorder Associated with Phosphorylation of Eukaryotic Initiation Factor 2 Alpha (eIF2α)

In some embodiments, the presently disclosed subject matter provides a method for treating a disease, condition, or disorder associated with phosphorylation of eukaryotic initiation factor 2 alpha (eIF2α), the method comprising administering to a subject in need of treatment thereof a therapeutically effective amount of a compound of formula (I).

In some embodiments, the disease, condition, or disorder associated with phosphorylation of eIF2α comprises a neurodegenerative disease. In certain embodiments, the neurodegenerative disease is selected from Alzheimer's disease, Parkinson disease, Creutzfeldt-Jakob disease, Huntington's disease, frontotemporal dementia (FTD), and amyotrophic lateral sclerosis (ALS).

In certain embodiments, the administering of a therapeutically effective amount of a compound of formula (I) inhibits microtubule affinity-regulating kinase 2 (MARK2) kinase activity. In particular embodiments, inhibiting MARK2 kinase activity reduces phosphorylation of eukaryotic initiation factor 2 alpha (eIF2α). In certain embodiments, reducing the phosphorylation of eIF2α reduces the phosphorylation of eIF2α-⁵¹S.

In certain embodiments, the phosphorylation of eIF2α is associated with a response to proteotoxic stress. In particular embodiments, the proteotoxic stress is associated with protein misfolding. In certain embodiments, phosphorylation of eIF2α is associated with regulating translation under stress.

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

As used herein, the term “inhibit,” and grammatical derivations thereof, refers to the ability of a presently disclosed compound, e.g., a presently disclosed compound of formula (I), to block, partially block, interfere, decrease, or reduce the growth of bacteria or a bacterial infection. Thus, one of ordinary skill in the art would appreciate that the term “inhibit” encompasses a complete and/or partial decrease in the growth of bacteria or a bacterial infection, e.g., a decrease by at least 10%, in some embodiments, a decrease by at least 20%, 30%, 50%, 75%, 95%, 98%, and up to and including 100%.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

The term “combination” is used in its broadest sense and means that a subject is administered at least two agents, more particularly a compound of formula (I) described herein and at least one other therapeutic agent. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the compounds described herein can be administered alone or in combination with adjuvants that enhance stability of the compounds, alone or in combination with one or more therapeutic agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of a compound described herein and at least one additional therapeutic agent can be varied so long as the beneficial effects of the combination of these agents are achieved. Accordingly, the phrase “in combination with” refers to the administration of a compound described herein and at least one additional therapeutic agent either simultaneously, sequentially, or a combination thereof. Therefore, a subject administered a combination of a compound described herein and at least one additional therapeutic agent can receive a compound and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the compound described herein and at least one additional therapeutic agent are administered simultaneously, they can be administered to the subject as separate pharmaceutical compositions, each comprising either a compound or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a compound described herein and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

$${Q}_{a} / {Q}_{A} + {Q}_{b} / {Q}_{B} = Synergy Index ( SI )$$

wherein:
   Q_{A} is the concentration of a component A, acting alone, which produced an end point in relation to component A;
   Qₐ is the concentration of component A, in a mixture, which produced an end point;
   Q_{B} is the concentration of a component B, acting alone, which produced an end point in relation to component B; and
   Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

### C. Pharmaceutical Compositions and Administration

In another aspect, the present disclosure provides a pharmaceutical composition including one compound described herein alone or in combination with one or more additional therapeutic agents in admixture with a pharmaceutically acceptable excipient. One of skill in the art will recognize that the pharmaceutical compositions include the pharmaceutically acceptable salts of the compounds described above. Pharmaceutically acceptable salts are generally well known to those of ordinary skill in the art, and include salts of active compounds which are prepared with relatively nontoxic acids or bases, depending on the particular substituent moieties found on the compounds described herein. When compounds of the present disclosure contain relatively acidic functionalities, base addition salts can be obtained by contacting the neutral form of such compounds with a sufficient amount of the desired base, either neat or in a suitable inert solvent or by ion exchange, whereby one basic counterion (base) in an ionic complex is substituted for another. Examples of pharmaceutically acceptable base addition salts include sodium, potassium, calcium, ammonium, organic amino, or magnesium salt, or a similar salt.

When compounds of the present disclosure contain relatively basic functionalities, acid addition salts can be obtained by contacting the neutral form of such compounds with a sufficient amount of the desired acid, either neat or in a suitable inert solvent or by ion exchange, whereby one acidic counterion (acid) in an ionic complex is substituted for another. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-toluenesulfonic, citric, tartaric, methanesulfonic, trifluoroacetic acid (TFA), and the like. Also included are salts of amino acids such as arginate and the like, and salts of organic acids like glucuronic or galactunoric acids and the like (see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Accordingly, pharmaceutically acceptable salts suitable for use with the presently disclosed subject matter include, by way of example but not limitation, acetate, benzenesulfonate, benzoate, bicarbonate, bitartrate, bromide, calcium edetate, camsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000). In therapeutic and/or diagnostic applications, the compounds of the disclosure can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

Depending on the specific conditions being treated, such agents may be formulated into liquid or solid dosage forms and administered systemically or locally. The agents may be delivered, for example, in a timed- or sustained-slow release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances, such as saline; preservatives, such as benzyl alcohol; absorption promoters; and fluorocarbons.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, the bioavailability of the compound(s), the adsorption, distribution, metabolism, and excretion (ADME) toxicity of the compound(s), and the preference and experience of the attending physician.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

### D. Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

While the following terms in relation to compounds of formula (I) are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

The terms substituted, whether preceded by the term “optionally” or not, and substituent, as used herein, refer to the ability, as appreciated by one skilled in this art, to change one functional group for another functional group on a molecule, provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted at one or more positions).

Where substituent groups or linking groups are specified by their conventional chemical formulae, written from left to right, they equally encompass the chemically identical substituents that would result from writing the structure from right to left, e.g., —CH₂O— is equivalent to —OCH₂—; —C(═O)O— is equivalent to —OC(═O)—; —OC(═O)NR— is equivalent to —NRC(═O)O—, and the like.

When the term “independently selected” is used, the substituents being referred to (e.g., R groups, such as groups R¹, R₂, and the like, or variables, such as “m” and “n”), can be identical or different. For example, both R₁ and R₂ can be substituted alkyls, or R₁ can be hydrogen and R₂ can be a substituted alkyl, and the like.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless otherwise explicitly defined, a “substituent group,” as used herein, includes a functional group selected from one or more of the following moieties, which are defined herein:

The term hydrocarbon, as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, isopropyl, cyclopropyl, allyl, vinyl, n-butyl, tert-butyl, ethynyl, cyclohexyl, and the like.

The term “alkyl,” by itself or as part of another substituent, means, unless otherwise stated, a straight (i.e., unbranched) or branched chain, acyclic or cyclic hydrocarbon group, or combination thereof, which may be fully saturated, mono- or polyunsaturated and can include di- and multivalent groups, having the number of carbon atoms designated (i.e., C₁₋₁₀ means one to ten carbons, including 1, 2, 3, 4, 5, 6, 7, 8, 9, and 10 carbons). In particular embodiments, the term “alkyl” refers to C₁₋₂₀ inclusive, including 1, 2, 3, 4, 5, 6, 7, 8, 9, 10, 11, 12, 13, 14, 15, 16, 17, 18, 19, and 20 carbons, linear (i.e., “straight-chain”), branched, or cyclic, saturated or at least partially and in some cases fully unsaturated (i.e., alkenyl and alkynyl) hydrocarbon radicals derived from a hydrocarbon moiety containing between one and twenty carbon atoms by removal of a single hydrogen atom.

Representative saturated hydrocarbon groups include, but are not limited to, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologs and isomers thereof.

“Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls. Representative C₁-C₈ alkyl groups include methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, and n-octyl.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, cyano, carboxyl, and mercapto.

The term “heteroalkyl,” by itself or in combination with another term, means, unless otherwise stated, a stable straight or branched chain having from 1 to 20 carbon atoms or heteroatoms or a cyclic hydrocarbon group having from 3 to 10 carbon atoms or heteroatoms, or combinations thereof, consisting of at least one carbon atom and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)— CH₃, O—CH₃, —O—CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)NR′, —NR′R″, —OR′, —SR, —S(O)R, and/or —S(O₂)R′. Where “heteroalkyl” is recited, followed by recitations of specific heteroalkyl groups, such as —NR′R or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, unsubstituted alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl, and fused ring systems, such as dihydro- and tetrahydronaphthalene, and the like.

The term “cycloalkylalkyl,” as used herein, refers to a cycloalkyl group as defined hereinabove, which is attached to the parent molecular moiety through an alkylene moiety, also as defined above, e.g., a C₁₋₂₀ alkylene moiety. Examples of cycloalkylalkyl groups include cyclopropylmethyl and cyclopentylethyl.

The terms “cycloheteroalkyl” or “heterocycloalkyl” refer to a non-aromatic ring system, unsaturated or partially unsaturated ring system, such as a 3- to 10-member substituted or unsubstituted cycloalkyl ring system, including one or more heteroatoms, which can be the same or different, and are selected from the group consisting of nitrogen (N), oxygen (O), sulfur (S), phosphorus (P), and silicon (Si), and optionally can include one or more double bonds.

The cycloheteroalkyl ring can be optionally fused to or otherwise attached to other cycloheteroalkyl rings and/or non-aromatic hydrocarbon rings. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocyclic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidinyl, piperazinyl, indolinyl, quinuclidinyl, morpholinyl, thiomorpholinyl, thiadiazinanyl, tetrahydrofuranyl, and the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

As used herein the terms “ ”and “bicycloheteroalkyl” refer to two cycloalkyl or cycloheteroalkyl groups that are bound to one another. Non-limiting examples include bicyclohexane and bipiperidine.

An unsaturated hydrocarbon has one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl.”

More particularly, the term “alkenyl” as used herein refers to a monovalent group derived from a C₂₋₂₀ inclusive straight or branched hydrocarbon moiety having at least one carbon-carbon double bond by the removal of a single hydrogen molecule. Alkenyl groups include, for example, ethenyl (i.e., vinyl), propenyl, butenyl, 1-methyl-2-buten-1-yl, pentenyl, hexenyl, octenyl, allenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight or branched C₂₋₂₀ hydrocarbon of a designed number of carbon atoms containing at least one carbon-carbon triple bond. Examples of “alkynyl” include ethynyl, 2-propynyl (propargyl), 1-propynyl, pentynyl, hexynyl, and heptynyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —CH₂CH₂CH₂CH₂—, —CH₂CH═CHCH₂—, —CH₂CsCCH₂—, —CH₂CH₂CH(CH₂CH₂CH₃)CH₂—, —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

The term “heteroalkylene” by itself or as part of another substituent means a divalent group derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms also can occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—.

The term “aryl” means, unless otherwise stated, an aromatic hydrocarbon substituent that can be a single ring or multiple rings (such as from 1 to 3 rings), which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent forms of aryl and heteroaryl, respectively.

For brevity, the term “aryl” when used in combination with other terms (e.g., aryloxy, arylthioxy, arylalkyl) includes both aryl and heteroaryl rings as defined above. Thus, the terms “arylalkyl” and “heteroarylalkyl” are meant to include those groups in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). However, the term “haloaryl,” as used herein is meant to cover only aryls substituted with one or more halogens.

Where a heteroalkyl, heterocycloalkyl, or heteroaryl includes a specific number of members (e.g. “3 to 7 membered”), the term “member” refers to a carbon or heteroatom.

Further, a structure represented generally by the formula:

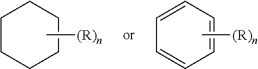

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

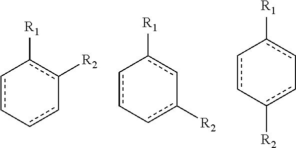

and the like.

A dashed line representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure.

The symbol ( [symbol] ) denotes the point of attachment of a moiety to the remainder of the molecule.

When a named atom of an aromatic ring or a heterocyclic aromatic ring is defined as being “absent,” the named atom is replaced by a direct bond.

Each of above terms (e.g., “alkyl,” “heteroalkyl,” “cycloalkyl, and “heterocycloalkyl”, “aryl,” “heteroaryl,” “phosphonate,” and “sulfonate” as well as their divalent derivatives) are meant to include both substituted and unsubstituted forms of the indicated group. Optional substituents for each type of group are provided below.

Substituents for alkyl, heteroalkyl, cycloalkyl, heterocycloalkyl monovalent and divalent derivative groups (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′, —OC(O)R′, —C(O)R′, —CO₂R′, —C(O)NR′R″, —OC(O)NR′R″, —NR″C(O)R′, —NR′—C(O)NR″R′, —NR″C(O)OR′, —NR—C(NR′R″)═NR′″, —S(O)R′, —S(O)₂R′, —S(O)₂NR′R″, —NRSO₂R′, —CN, CF₃, fluorinated C₁₋₄ alkyl, and —NO₂ in a number ranging from zero to (2m′+1), where m′ is the total number of carbon atoms in such groups. R′, R″, R′″ and R″″ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. As used herein, an “alkoxy” group is an alkyl attached to the remainder of the molecule through a divalent oxygen. When a compound of the disclosure includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl groups above, exemplary substituents for aryl and heteroaryl groups (as well as their divalent derivatives) are varied and are selected from, for example: halogen, —OR′, —NR′R″, —SR′, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R′″)═NR′″, —NR—C(NR′R″)═NR′″—S(O)R′, —S(O)₂R′, —S(O)₂NR′R″, —NRSO₂R′, —CN and —NO₂, —R′, —N₃, —CH(Ph)₂, fluoro(C₁₋₄)alkoxo, and fluoro(C₁₋₄)alkyl, in a number ranging from zero to the total number of open valences on aromatic ring system; and where R′, R″, R′″ and R″″ may be independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes more than one R group, for example, each of the R groups is independently selected as are each R′, R″, R′″ and R″″ groups when more than one of these groups is present.

Two of the substituents on adjacent atoms of aryl or heteroaryl ring may optionally form a ring of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4.

One of the single bonds of the new ring so formed may optionally be replaced with a double bond. Alternatively, two of the substituents on adjacent atoms of aryl or heteroaryl ring may optionally be replaced with a substituent of the formula —(CRR′)₅—X′— (C″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R′″ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocyclic, heterocyclic, or aromatic heterocyclic group as defined herein). As such, the term “acyl” specifically includes arylacyl groups, such as a 2-(furan-2-yl)acetyl)- and a 2-phenylacetyl group. Specific examples of acyl groups include acetyl and benzoyl. Acyl groups also are intended to include amides, —RC(═O)NR′, esters, —RC(═O)OR′, ketones, —RC(═O)R′, and aldehydes, —RC(═O)H.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl, i.e., C₆H₅—CH₂—O—. An aralkyloxyl group can optionally be substituted.

“Alkoxycarbonyl” refers to an alkyl-O—C(═O)— group. Exemplary alkoxycarbonyl groups include methoxycarbonyl, ethoxycarbonyl, butyloxycarbonyl, and tert-butyloxycarbonyl.

“Aryloxycarbonyl” refers to an aryl-O—C(═O)— group. Exemplary aryloxycarbonyl groups include phenoxy- and naphthoxy-carbonyl.

“Aralkoxycarbonyl” refers to an aralkyl-O—C(═O)— group. An exemplary aralkoxycarbonyl group is benzyloxycarbonyl.

“Carbamoyl” refers to an amide group of the formula —C(═O)NH2. “Alkylcarbamoyl” refers to a R′RN—C(═O)— group wherein one of R and R′ is hydrogen and the other of R and R′ is alkyl and/or substituted alkyl as previously described.

“Dialkylcarbamoyl” refers to a R′RN—C(═O)— group wherein each of R and R′ is independently alkyl and/or substituted alkyl as previously described.

The term carbonyldioxyl, as used herein, refers to a carbonate group of the formula —O—C(═O)—OR.

“Acyloxyl” refers to an acyl-O— group wherein acyl is as previously described.

The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR′ wherein R′ is an alkyl group, as previously defined; whereas the term dialkylamino refers to a group having the structure —NR′R″, wherein R′ and R″ are each independently selected from the group consisting of alkyl groups. The term trialkylamino refers to a group having the structure —NR′R″R′″, wherein R′, R″, and R′″ are each independently selected from the group consisting of alkyl groups. Additionally, R′, R″, and/or R′″ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, isopropylamino, piperidino, trimethylamino, and propylamino.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH— group wherein acyl is as previously described. “Aroylamino” refers to an aroyl-NH— group wherein aroyl is as previously described.

The term “carbonyl” refers to the —C(═O)— group, and can include an aldehyde group represented by the general formula R—C(═O)H.

The term “carboxyl” refers to the —COOH group. Such groups also are referred to herein as a “carboxylic acid” moiety.

The term “cyano” refers to the —C—N group.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C₁₋₄)alkyl” is mean to include, but not be limited to, trifluoromethyl, 2,2,2-trifluoroethyl, 4-chlorobutyl, 3-bromopropyl, and the like.

The term “hydroxyl” refers to the —OH group.

The term “hydroxyalkyl” refers to an alkyl group substituted with an —OH group.

The term “mercapto” refers to the —SH group.

The term “oxo” as used herein means an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula —SH.

More particularly, the term “sulfide” refers to compound having a group of the formula —SR.

The term “sulfone” refers to compound having a sulfonyl group —S(O₂)R.

The term “sulfoxide” refers to a compound having a sulfinyl group —S(O)R The term ureido refers to a urea group of the formula —NH—CO—NH₂.

Throughout the specification and claims, a given chemical formula or name shall encompass all tautomers, congeners, and optical- and stereoisomers, as well as racemic mixtures where such isomers and mixtures exist.

Certain compounds of the present disclosure may possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as D- or L- for amino acids, and individual isomers are encompassed within the scope of the present disclosure. The compounds of the present disclosure do not include those which are known in art to be too unstable to synthesize and/or isolate. The present disclosure is meant to include compounds in racemic, scalemic, and optically pure forms. Optically active (R)- and (S)-, or D- and L-isomers may be prepared using chiral synthons or chiral reagents, or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Unless otherwise stated, structures depicted herein are also meant to include compounds which differ only in the presence of one or more isotopically enriched atoms. For example, compounds having the present structures with the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are encompassed within the scope of the present disclosure.

The compounds of the present disclosure may exist as salts. The present disclosure includes such salts. Examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent or by ion exchange. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

The term “protecting group” refers to chemical moieties that block some or all reactive moieties of a compound and prevent such moieties from participating in chemical reactions until the protective group is removed, for example, those moieties listed and described in T. W. Greene, P.G.M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups. For example, protective groups can be removed by acid, base, and hydrogenolysis. Groups such as trityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as tert-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be blocked with oxidatively-removable protective groups such as 2,4-dimethoxybenzyl, while co-existing amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium(O)— catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typical blocking/protecting groups include, but are not limited to the following moieties:

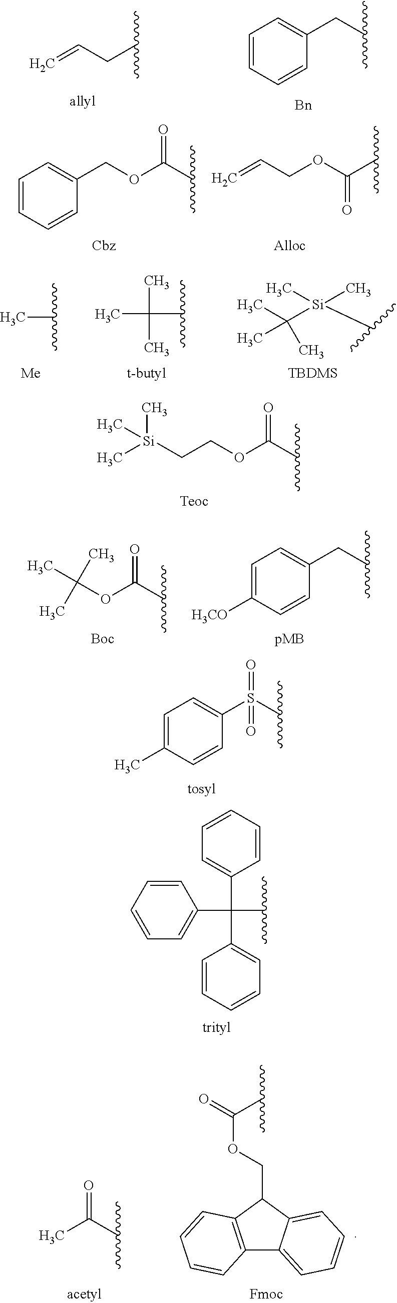

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments 10%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

MARK2 Phosphorylates eIF2α in Response to Proteotoxic Stress

### 1.1 Overview

The regulation of protein synthesis is essential for maintaining cellular homeostasis, especially during stress responses, and its dysregulation could underlie the development of human diseases. A critical step during translation regulation is the phosphorylation of eukaryotic initiation factor 2 alpha (eIF2α).

The presently disclosed subject matter provides the identification of microtubule affinity-regulating kinase 2 (MARK2), a serine/threonine kinase previously implicated in the regulation of microtubule stability, Suzuki et al., 2004; Hurov et al., 2004, as a direct kinase of eIF2α under conditions of protein misfolding stress. MARK2 itself is a substrate of protein kinase C delta (PKCδ), a member of the PKC kinase family that has a conserved role in regulating cell polarity and signaling pathways. Suzuki et al., 2004; Hurov et al., 2004; Jiang et al., 2014.

Both MARK2 and PKCδ are phosphorylated under proteotoxic stress, and both kinases are required for the stress-induced phosphorylation of eIF2α. PKCδ serves as a sensor for protein misfolding stress through its dynamic interaction with the molecular chaperone HSP90. MARK2 and PKCδ are also activated in the nervous systems of mouse models of SOD1-linked ALS and in patients with ALS. These results reveal a cytosolic signaling pathway that regulates eIF2α phosphorylation and protein synthesis and may have important implications for our understanding of normal cellular stress responses and the pathogenic process in proteotoxicity-related neurodegenerative diseases.

### 1.2 Results

1.2.1 MARK2 is a direct kinase for eIF2α

Phosphorylation of eIF2α is a key step in the translational attenuation that occurs in response to a variety of stresses in mammalian cells. Dever, 2002. To identify previously unrecognized eIF2α kinases, we searched a protein array dataset that suggested potential kinase and substrate relationships using microarrays composed of 4,191 unique human full-length proteins subjected to phosphorylation reactions with over 200 purified human kinases. Newman et al., 2013; Hu et al., 2009.

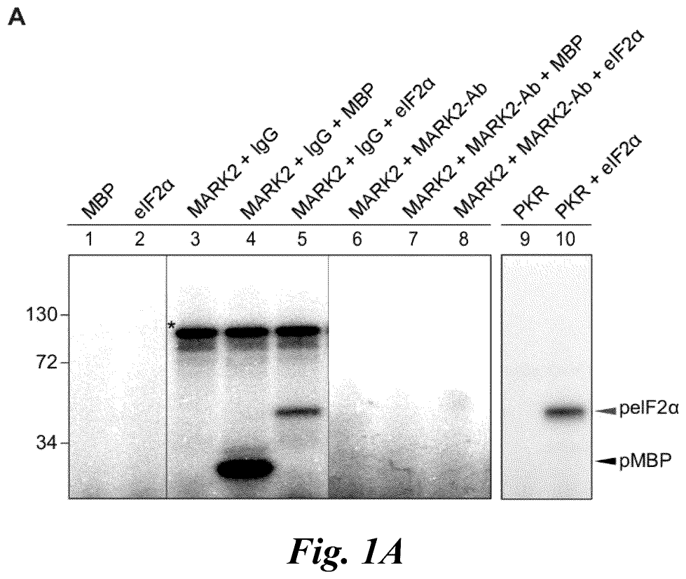

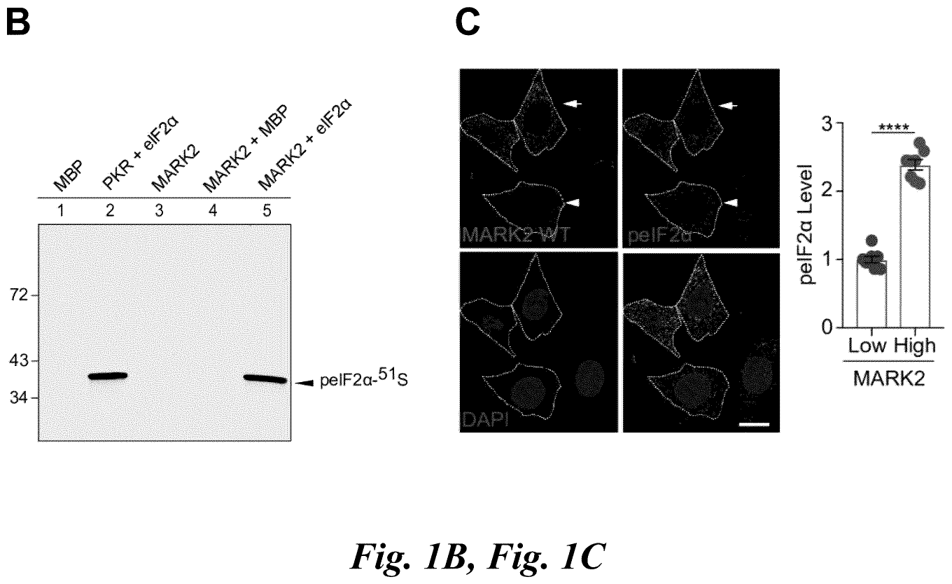

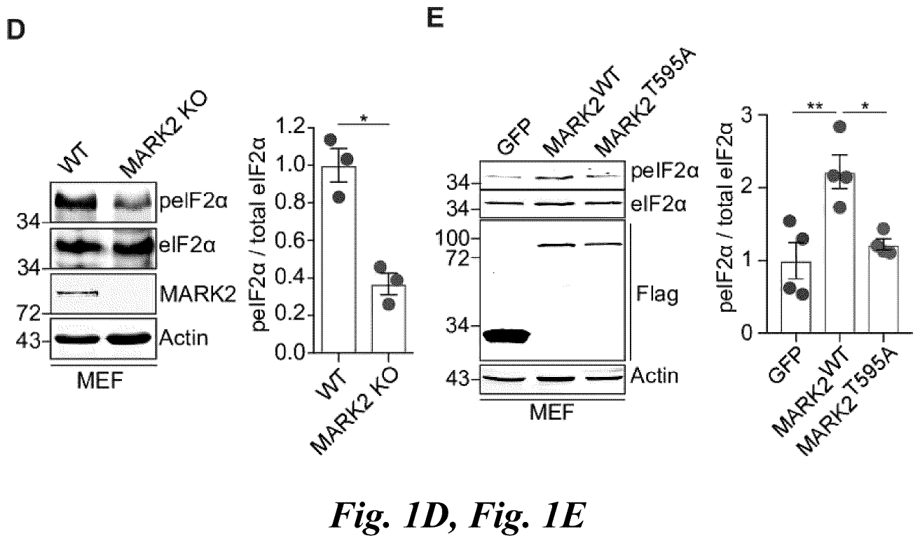

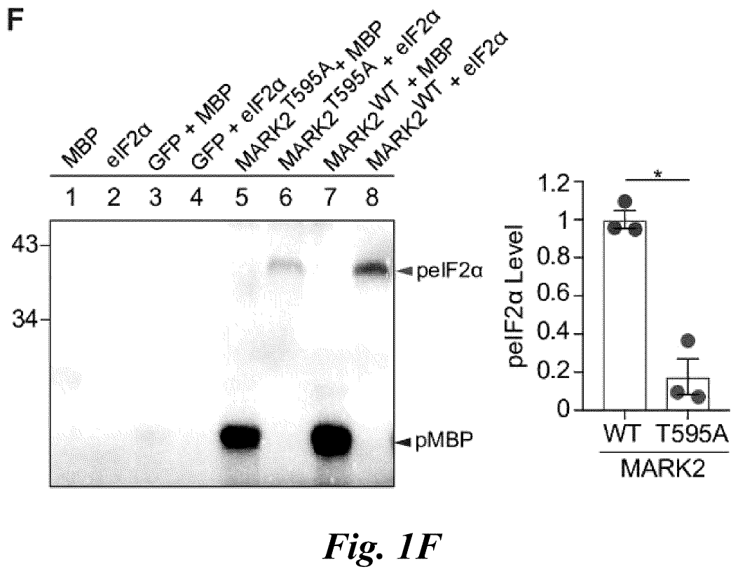

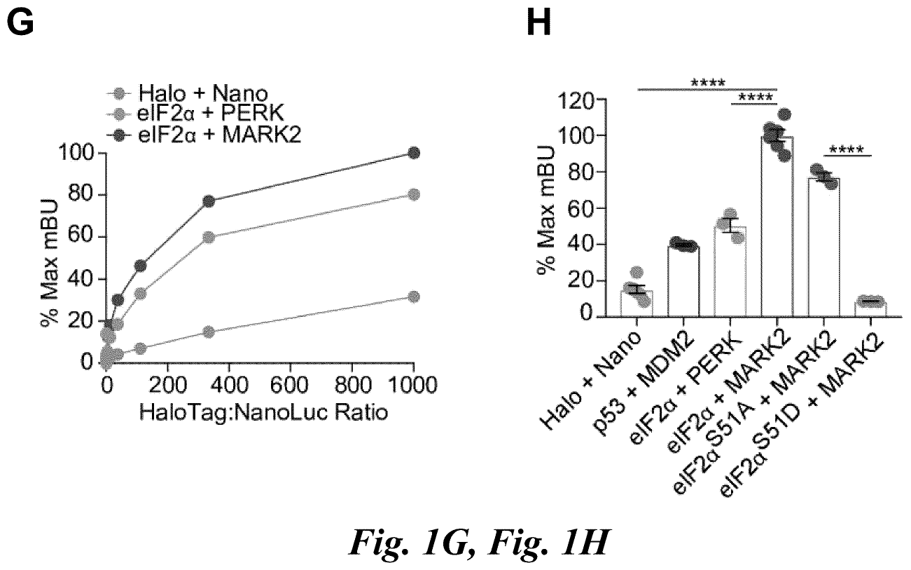

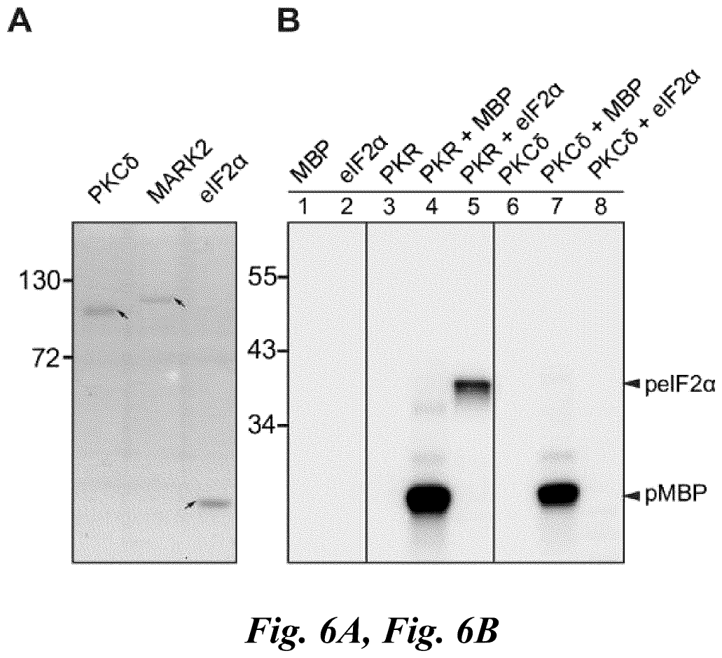

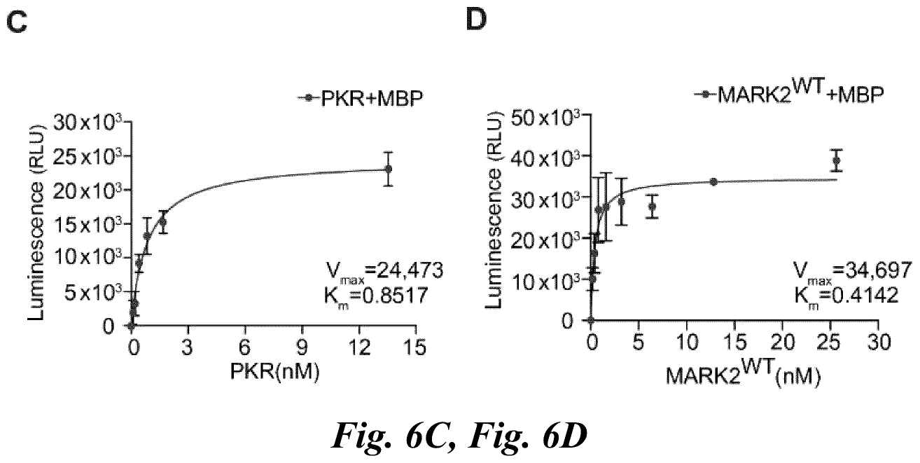

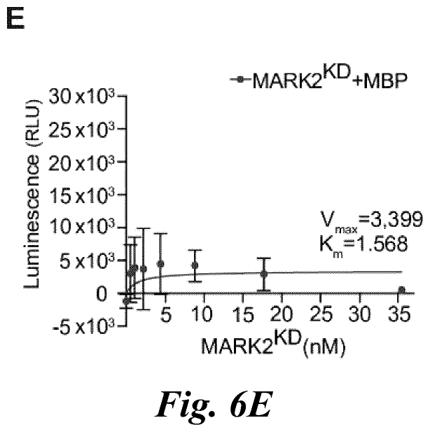

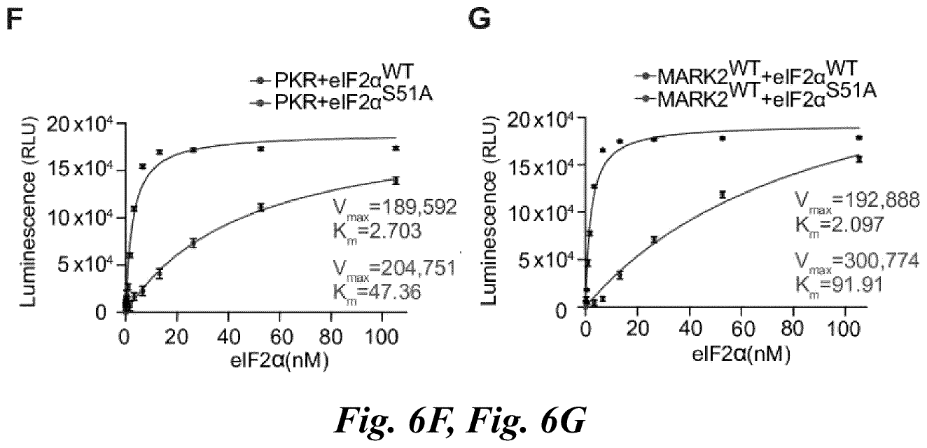

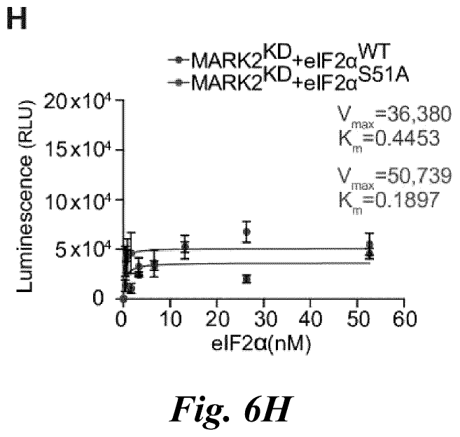

The protein array screen suggested at least four candidate kinases for eIF2α: protein tyrosine kinase 2 beta (PYK2), TTK protein kinase (TTK), bone morphogenetic protein receptor type 1A (BMPR1A), and MARK2. To determine which of these candidate kinases is capable of phosphorylating eIF2α, we performed in vitro kinase assays with radiolabeled ATP and proteins purified from Sf9 insect cells, including the eIF2α substrate and each of the 4 candidate kinases. Only MARK2 showed kinase activity, phosphorylating eIF2α in vitro (FIG. 1A, lane 5 and FIG. 6A and FIG. 6B). Myelin basic protein (MBP), a common substrate for diverse kinases, and PKR, a positive control kinase, were used to confirm that all the tested kinases were enzymatically active. Notably, the phosphorylation of eIF2α by MARK2 was completely blocked by a MARK2-specific antibody but not by an immunoglobulin G (IgG) control (FIG. 1A, lane 8), confirming that the observed activity for the eIF2α kinase was specifically associated with the MARK2 protein.

To characterize the kinase activity of MARK2 toward eIF2α, we purified a series of WT and mutant MARK2 and eIF2α proteins using an E. coli expression system and performed in vitro enzyme kinetics analyses. The Kinase-Glo assay was used to measure kinase activities by quantifying ATP consumption via luminescent signals. First, by using MBP as a shared substrate, we observed that MARK2 and the positive control eIF2α kinase PKR showed similar reactivity as shown in Michaelis-Menten kinetics curves (FIG. 6C and FIG. 6D). Additionally, we generated a kinase-dead mutant MARK2^{KD}, which lacks the catalytic domain, Drewes et al., 1997, as a negative control and confirmed the absence of kinase activity for this mutant (FIG. 6E). Then, using the Km concentrations of MARK2 and PKR established above (FIG. 6C and FIG. 6D), we studied the kinetics of their kinase activity toward eIF2α. We found that MARK2 exhibited a robust kinase activity for eIF2α comparable to that of PKR, as evidenced by the similarity of Km and Vmax values in Michaelis-Menten kinetics curves between the 2 sets of reactions (FIG. 6F and FIG. 6G). As expected, the kinase-dead MARK2^{KD} mutant did not show activity toward eIF2α (FIG. 6H). Translational control via the phosphorylation of eIF2α at serine 51 is a point of convergence for integrated stress response pathways. Dever, 2002.

Using a phosphorylation-dependent antibody against phospho-eIF2α-⁵¹S, we showed that the radiolabeled phospho-eIF2α signal seen in the kinase assay with MARK2 was positively recognized by the antibody against the phosphorylated eIF2α-⁵¹S (FIG. 1B). To further validate the phosphorylation of eIF2α by MARK2 at serine 51, we purified the phosphor-null mutant eIF2α^{S51A}. Using the in vitro kinase assay based on ATP radiolabeling and gel electrophoresis, we confirmed that eIF2α^{S51A} was not phosphorylated, when compared to the WT form of the substrate, by either PKR or MARK2 (FIG. 6I). The kinase-dead MARK2K mutant did not show activity toward eIF2α^{WT} or eIF2α^{S51A} (FIG. 6I). Consistently, in the kinetics analysis, the eIF2α^{S51A} mutant protein showed substantially lower reactivity for PKR or MARK2 than WT eIF2α, as evidenced by the curve slopes and Km values (FIG. 6F and FIG. 6G). Together, these results demonstrate that MARK2 directly phosphorylates eIF2α at serine 51.

1.2.2 MARK2 is a Kinase for eIF2α in Mammalian Cells

To study the physiologically relevant kinase activity of MARK2 in vivo, we analyzed the kinase activity of MARK2 on eIF2α in mammalian cells. Using mouse embryonic fibroblasts (MEFs), we compared cells expressing different levels of MARK2 for their correlation with the levels of phosphorylated eIF2α-⁵¹S: The cells with relatively higher levels of cytoplasmic MARK2 showed higher levels of phosphorylated eIF2α-⁵¹S, whereas the neighboring cells with less MARK2 showed lower levels of phosphorylated eIF2α-⁵¹S, as demonstrated by immunofluorescent staining for both MARK2 and eIF2α (FIG. 1C), suggesting that MARK2 positively regulates eIF2α phosphorylation in the cells. Next, we examined MARK2-mediated regulation of eIF2α phosphorylation by immunoblot analysis. Using MEFs in which the MARK2 locus was disrupted by the deletion of exons 2 to 4, Hurov et al., 2001, we found that the absence of MARK2 resulted in a significant reduction in the level of phosphorylated eIF2α-⁵¹S, without any detectable change in the level of total eIF2α protein (FIG. 1D). This finding was validated in human HAP1 cells in between MARK2 and eIF2α in the live cells. The MARK2 and eIF2α pair showed a hyperbolic curve, indicating that the energy transfer value reached a maximum when all the donors were saturated with the acceptors (FIG. 1G). Interestingly, the interaction between MARK2 and eIF2α indicated by the BRET signal of the pair was stronger than that between the known kinase PERK and eIF2α, which itself was stronger than the nonspecific interaction between unfused NanoLuc and HaloTag, in the donor saturation assay (FIG. 1G). Moreover, the interaction between MARK2 and eIF2α was stronger than that of another positive control interacting pair, p53 and MDM2, in the quantitative NanoBRET assay (FIG. 1H).

To determine how phosphorylation of eIF2α at the serine 51 site affects its interaction with MARK2, we generated the phosphor-null and phosphor-mimicking mutants S51A and S51D, respectively, for eIF2α and subjected them to the NanoBRET assay. Whereas the eIF2α^{S51A} mutant retained much of the interaction with MARK2, the eIF2α^{S51D}mutant showed no interaction with MARK2 (FIG. 1H), suggesting that the phosphorylation event diminishes the interaction between MARK2 and eIF2α. Collectively, these results support the notion that MARK2 is a direct and specific kinase of eIF2α at its serine 51 site both in vitro and in vivo.

1.2.3 MARK2 Mediates eIF2α Phosphorylation Independently of Previously Known Kinases

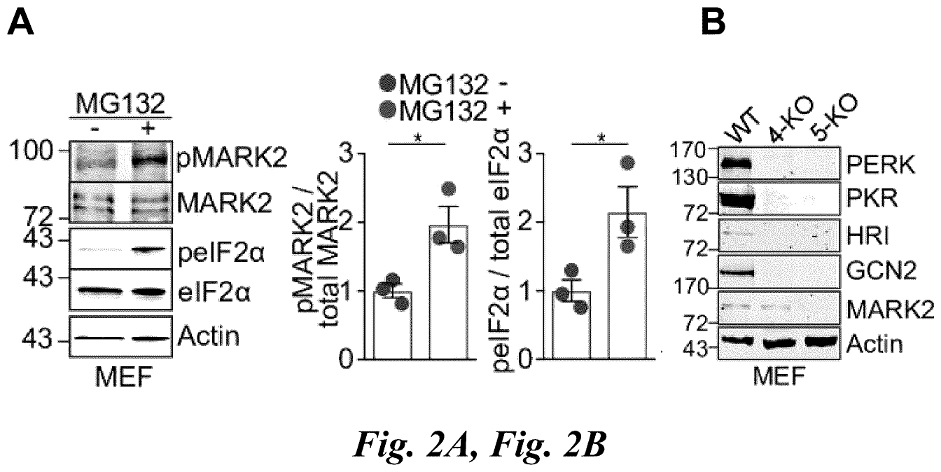

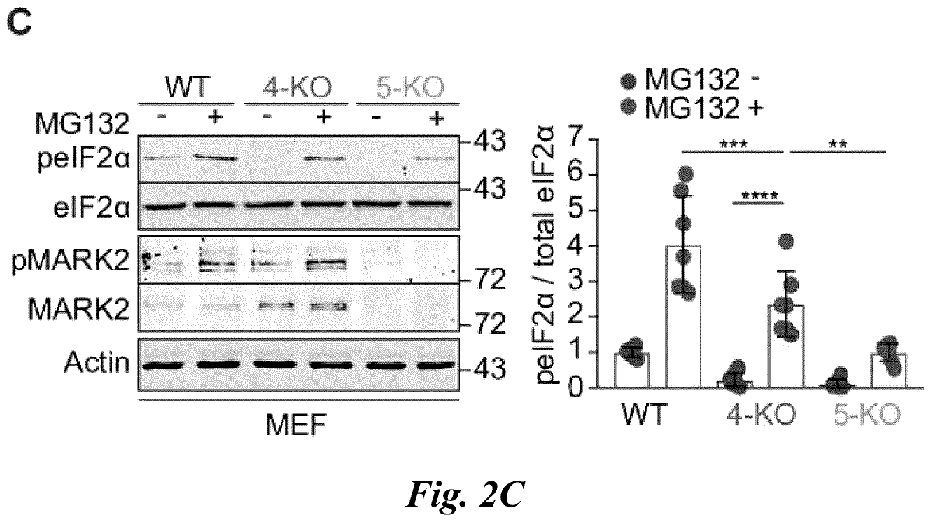

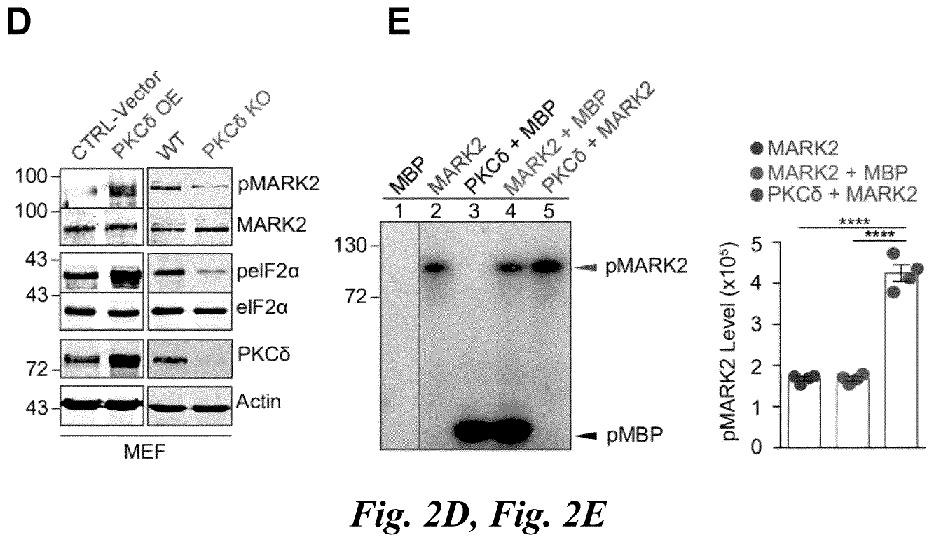

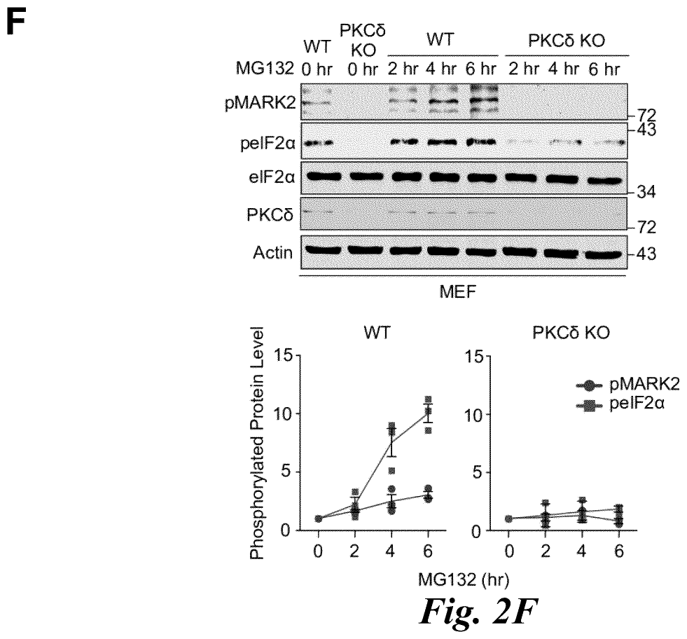

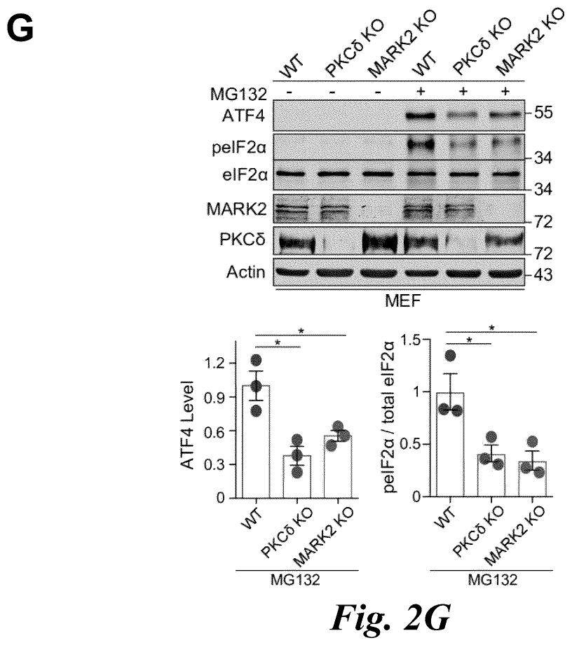

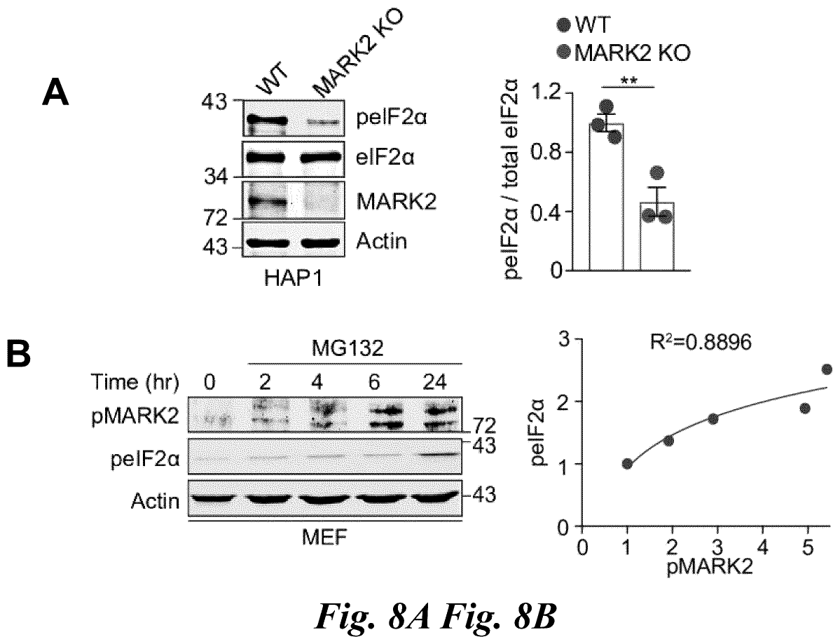

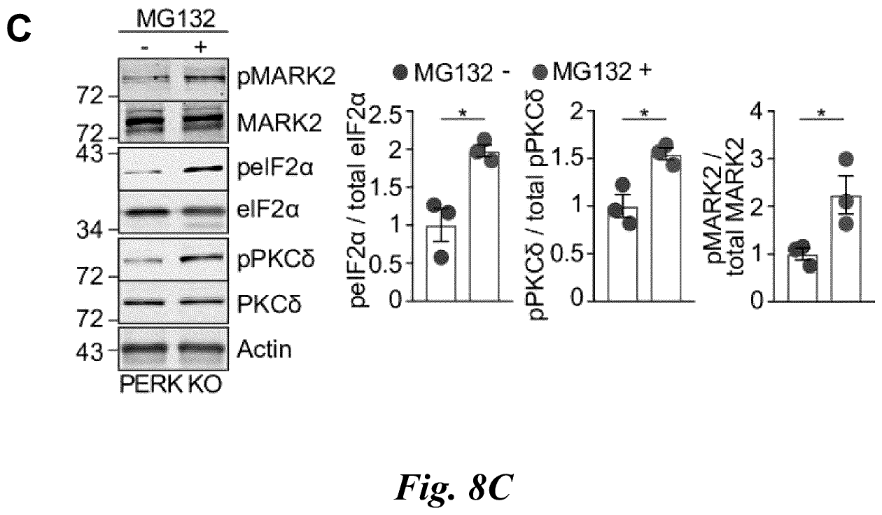

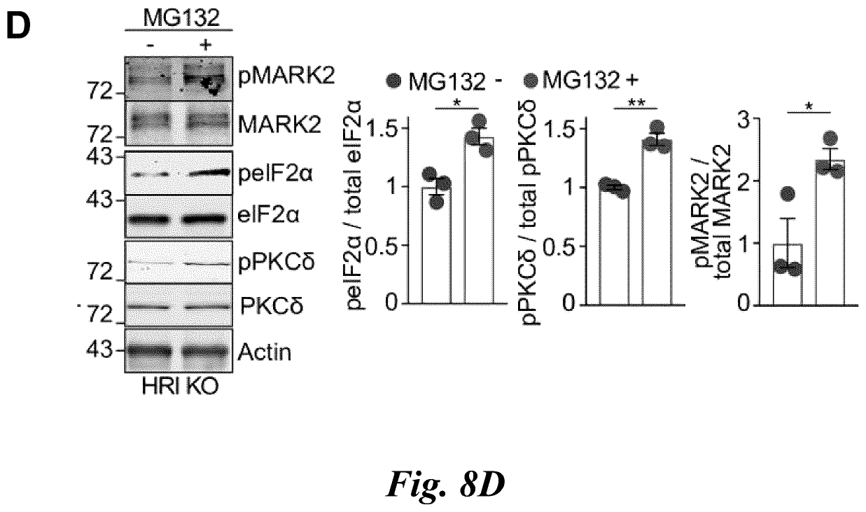

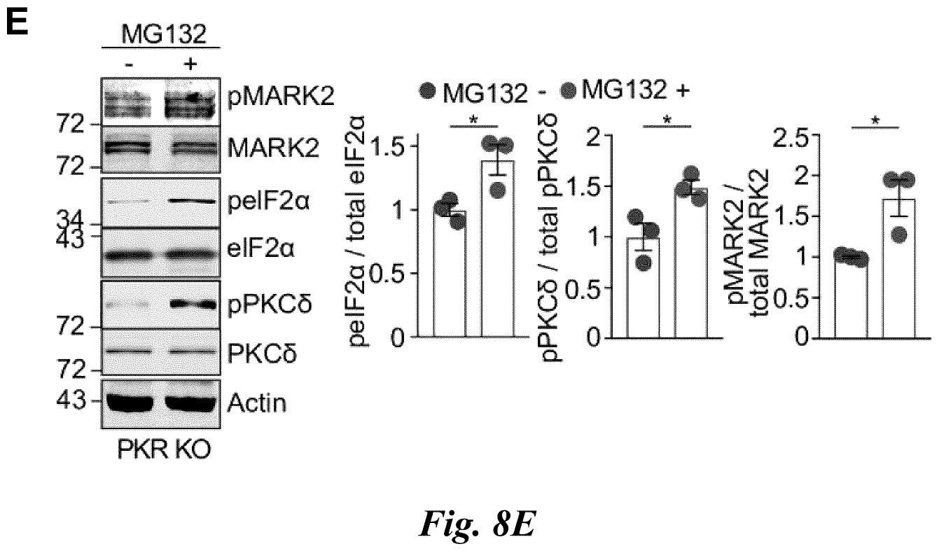

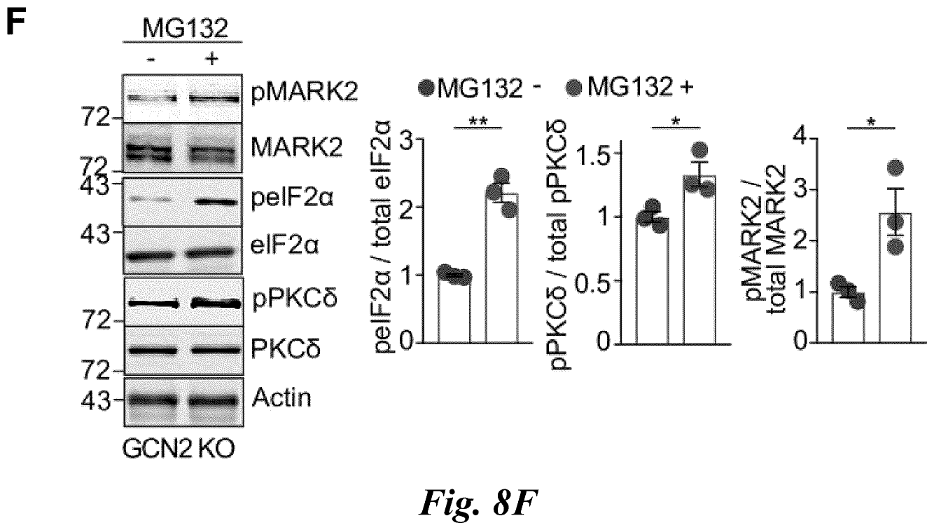

Since the phosphorylation of MARK2 at threonine 595 is required for its positive regulation of eIF2α phosphorylation (FIG. 1E), we asked whether this form of phosphorylated MARK2 is regulated upon cytosolic protein misfolding stress. To induce the protein misfolding stress, we treated MEF cells with the proteasome inhibitor MG132, and it elicited a substantial increase in the levels of phosphorylated MARK2-⁵⁹⁵T, as well as a corresponding increase in the levels of phosphorylated eIF2α-⁵¹S (FIG. 2A). In a time course study using the treatment with 500 nM MG132 for 24 h, the proteotoxic stressor led to a time-dependent increase in the levels of phosphorylated MARK2-⁵⁹⁵T, together with concomitantly increased levels of phosphorylated eIF2α-⁵¹S (FIG. 8B). These data suggest that the MARK2-eIF2α signaling pathway is activated in response to the proteotoxic stress.

Next we tested the activation of MARK2 by proteotoxic stress in the absence of PERK, HRI, PKR, or GCN2. In all four types of knockout MEFs lacking each of the four kinases, MARK2 was activated under the MG132-induced stress, as indicated by the increased phosphorylation at its threonine 595 site (FIG. 8C-FIG. 8F). Accordingly, the phosphorylation of eIF2α-⁵¹S also was significantly increased (FIG. 8C-FIG. 8F). These data indicate that the activation of the MARK2-eIF2α pathway by the protein misfolding stress does not require any of the four previously known eIF2α kinases.

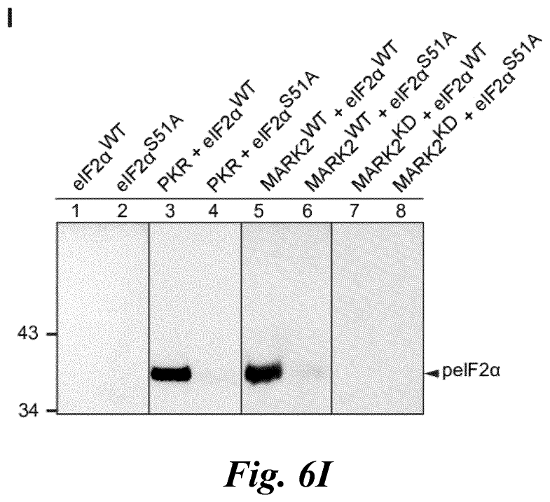

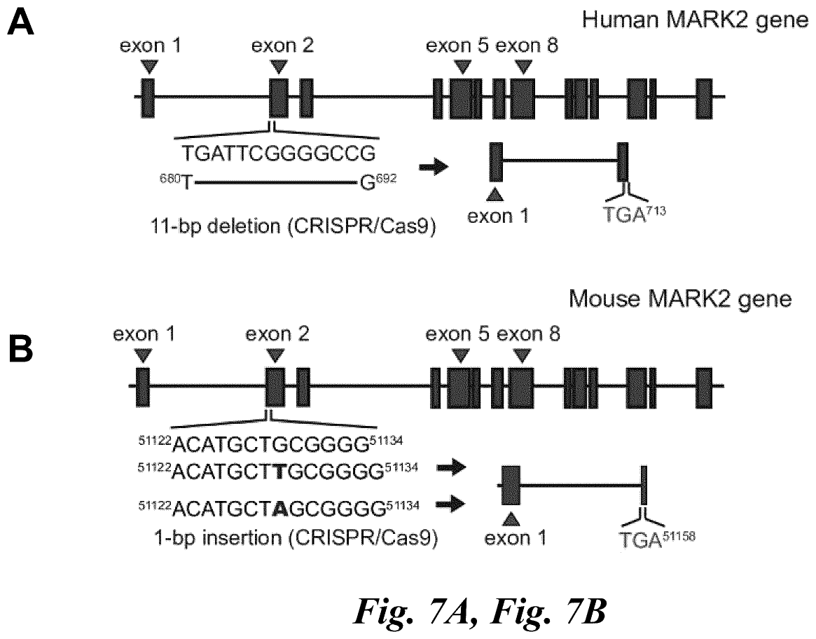

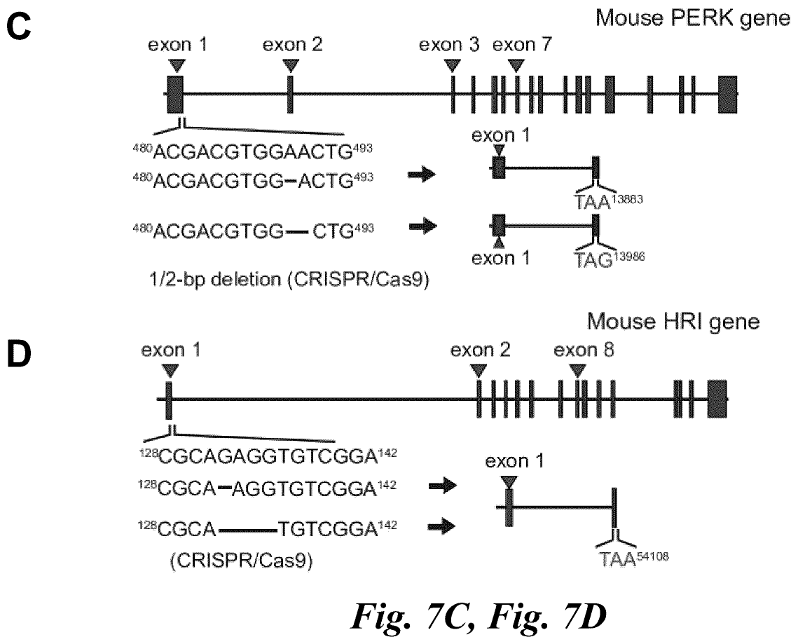

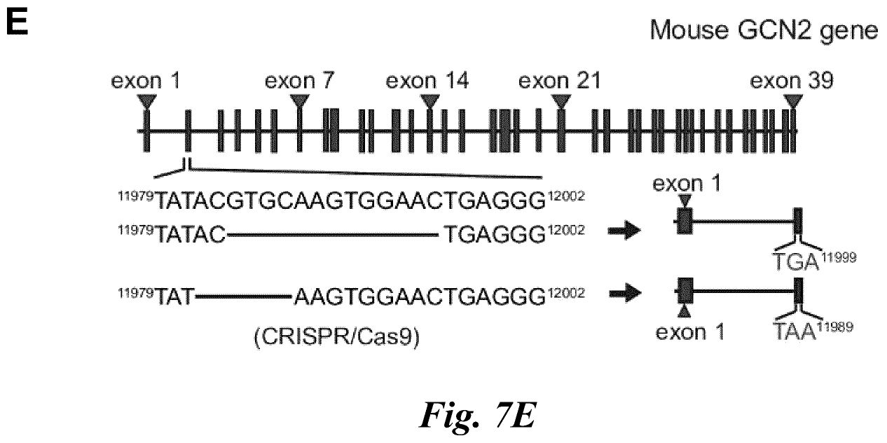

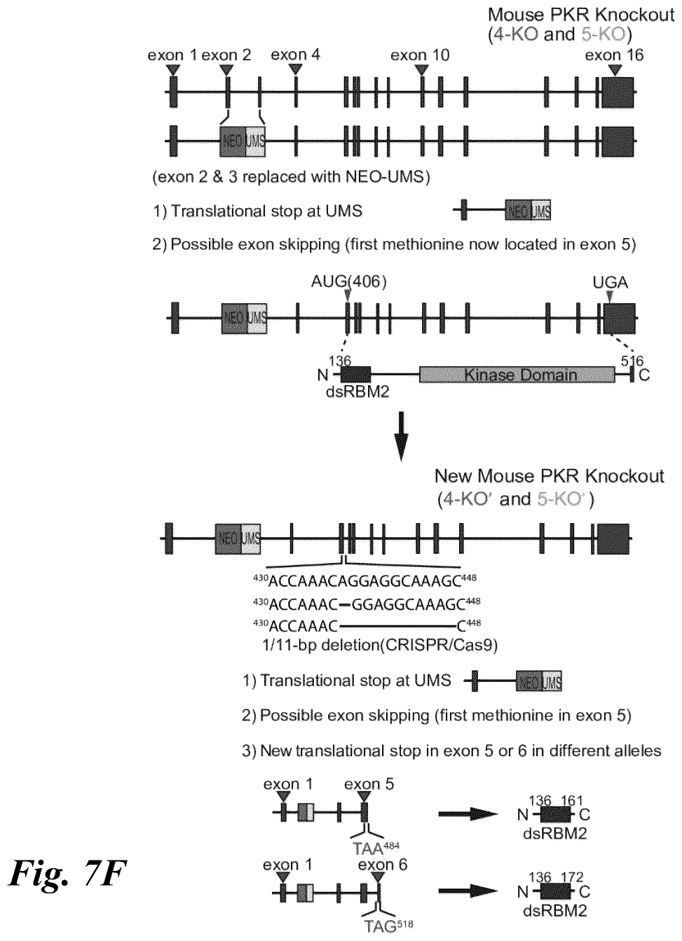

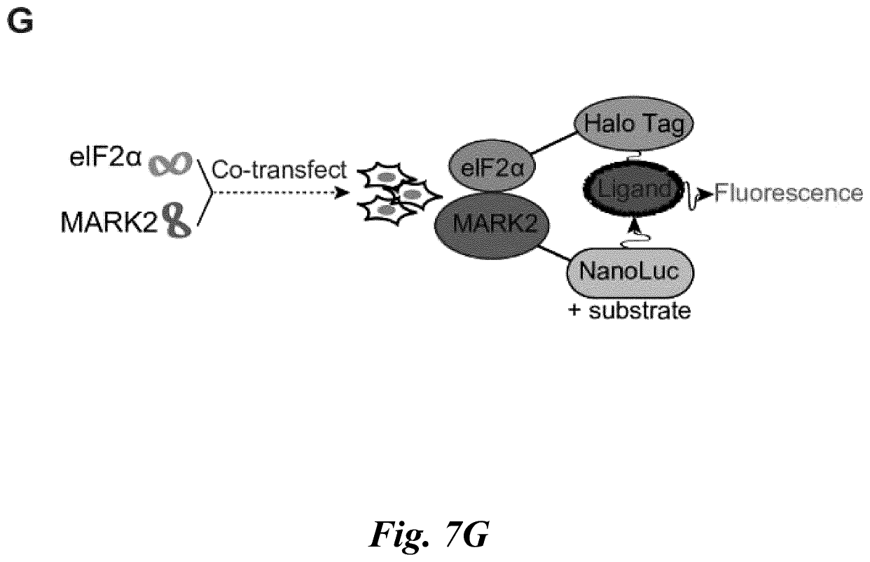

To further demonstrate that MARK2 alone is sufficient to promote the phosphorylation of eIF2α in the absence of all 4 previously known eIF2α kinases, we used multiplex CRISPR-Cas9 gene editing to knock out PERK, GCN2, and HRI in an existing PKR-knockout MEF line, Yang et al., 1995, creating 4-KO MEF lines (FIG. 2B and FIG. 7C-FIG. 7F).

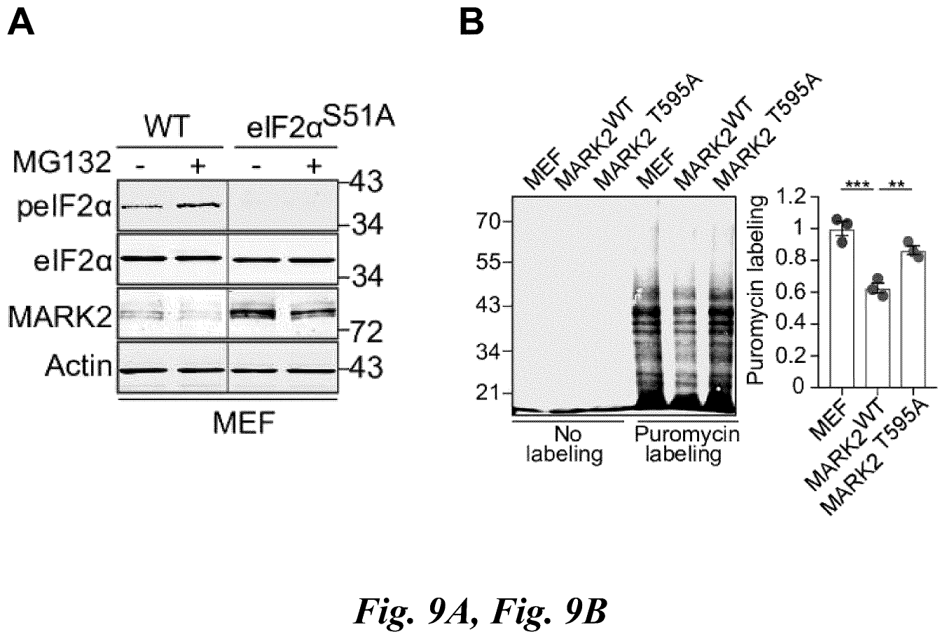

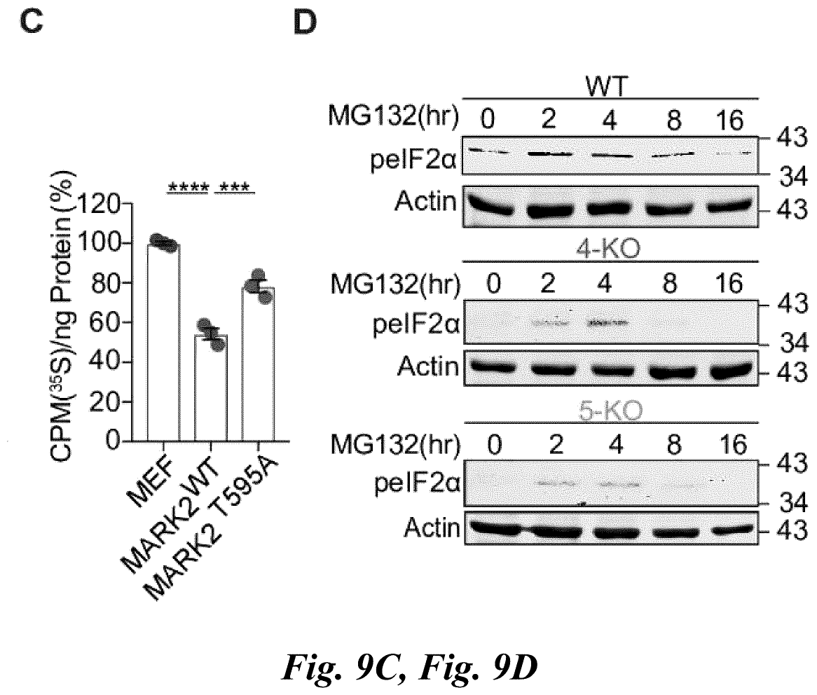

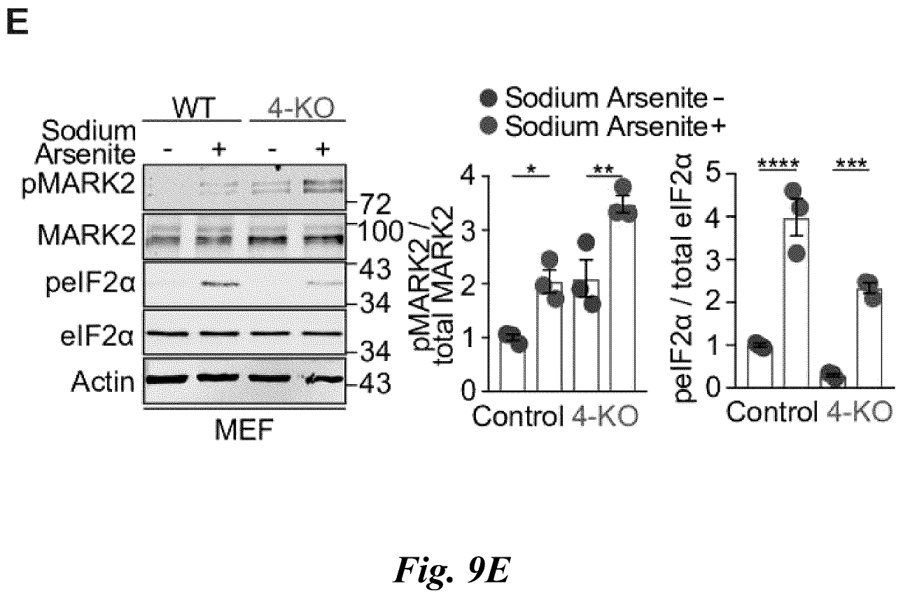

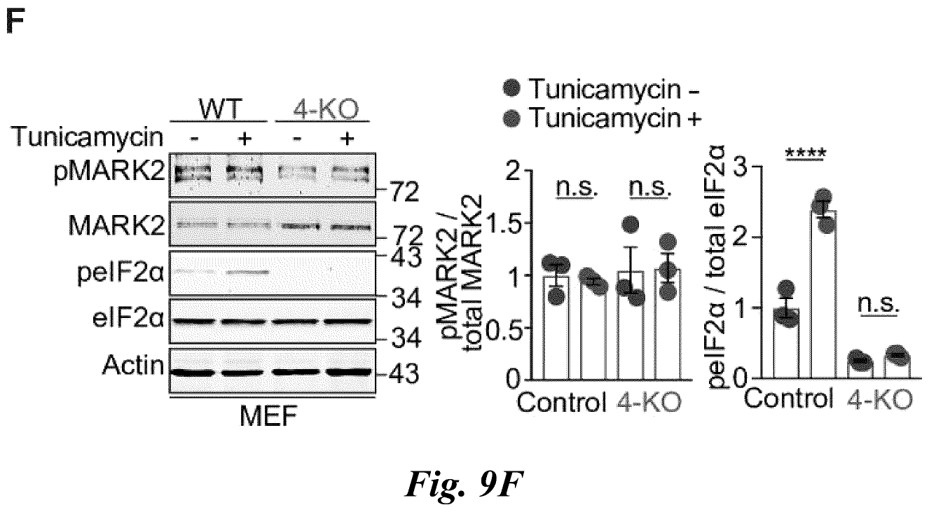

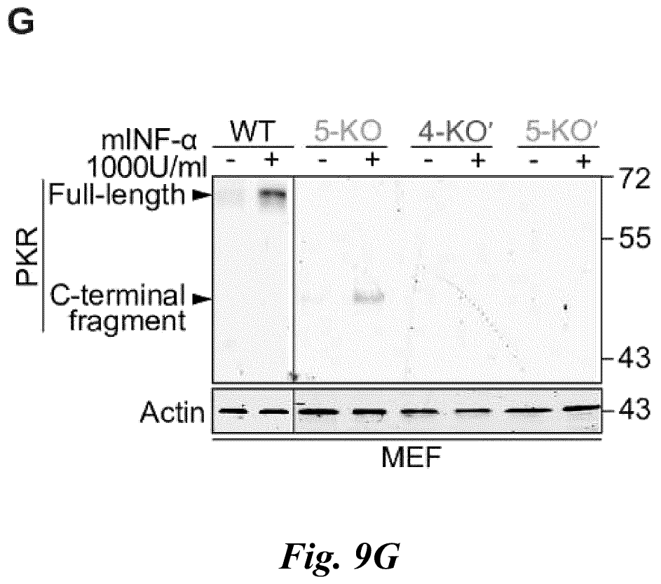

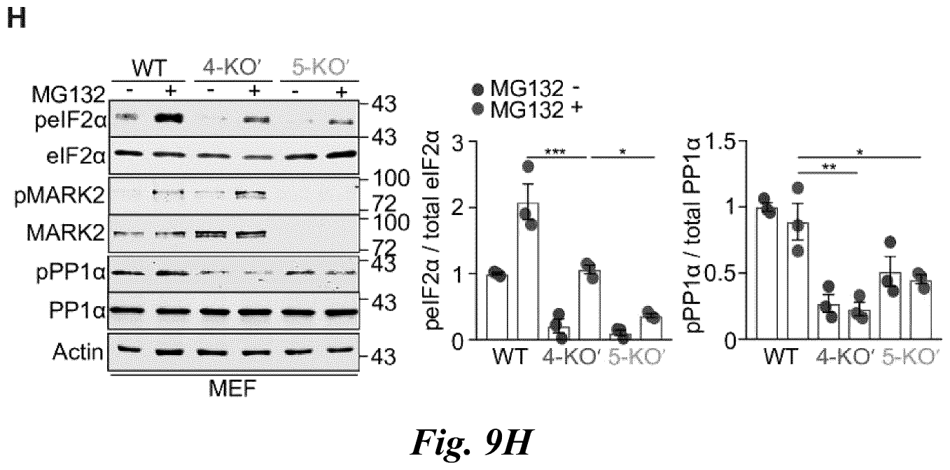

Next, we applied the proteasome inhibitor MG132 to the 4-KO MEF cells and found that the stress response, as indicated by eIF2α phosphorylation, was still intact. The phosphorylation of eIF2α-⁵¹S induced by the proteotoxic stress was lower in the 4-KO MEFs than in the WT MEFs (FIG. 2C); however, when compared to the unstressed cells, the phosphorylation of eIF2α in the absence of all 4 previously established kinases remained clearly detectable (FIG. 2C). In the 4-KO MEFs, the stress-induced change in the levels of phosphorylated eIF2α-⁵¹S was correlated with the increase in the level of MARK2-⁵⁹⁵T (FIG. 2C). To test whether MARK2 mediates the phosphorylation of eIF2α, we created independent 5-KO MEF lines by knocking out MARK2 in the 4-KO MEFs (FIG. 2C). When the 5-KO MEFs were treated with MG132, the phosphorylation of eIF2α was still enhanced, but the increase was significantly less than that in the 4-KO MEFs (FIG. 2C), indicating that MARK2 is capable of promoting eIF2α phosphorylation independently of the 4 previously known kinases. The condition of MG132 treatment at 20 μM for 4 h was chosen for optimal induction of eIF2α phosphorylation in the analysis of these MEFs (FIG. 9D). To test whether activation of MARK2 also can be induced by other types of stress, we subjected the cells to oxidative stress, such as sodium arsenite treatment, or ER stress, such as tunicamycin treatment. The sodium arsenite treatment, known to cause protein damages throughout the cell, was able to induce the phosphorylation of MARK2-⁵⁹⁵T and eIF2α-⁵¹S in both WT MEFs and those lacking the four previously known kinases (FIG. 9E), consistent with the activation of MARK2 by proteotoxic stress. In comparison, the tunicamycin treatment, known to activate PERK, has no effect on the phosphorylation of MARK2-⁵⁹⁵T, induced the phosphorylation of eIF2α-⁵¹S in WT MEFs but not in the cells lacking the 4 previously known kinases including PERK (FIG. 9F), consistent with the notion that MARK2 and ER stress act via independent pathways to regulate eIF2α phosphorylation. It was reported that the PKR KO MEF line that we used to generate the 4-KO and 5-KO cells expresses a remnant C-terminal fragment of the PKR protein. Yang et al., 1995; Balzis et al., 2002.

We used CRISPR editing to create additional deletion mutations in the PKR gene in the exiting 4-KO and 5-KO cells (FIG. 7F) and confirmed the disruption of the remnant C-terminal fragment, as validated by the absence of its expression under interferon-α stimulation, in the newly generated 4-KGO and 5-KGO MEF lines (FIG. 9G). Examination of the phosphorylation of eIF2α-⁵¹S induced by MG132 in the 4-KO0 and 5-KO0 MEFs showed similar results as those in the 4-KO and 5-KO cells (FIG. 9H), confirming the MARK2-dependent phosphorylation of eIF2α in the absence of the 4 previously known kinases. Furthermore, we examined the activity of protein phosphatase 1 (PP1), which catalyzes the dephosphorylation of eIF2α. Novoa et a 1., 2001. PP1α is phosphorylated at threonine 320 (320T), which inhibits its phosphatase activity. Kwon et al, 1997.

We observed a decrease in the phosphorylation of PP1α-³²⁰T in 4-KO0 and 5-KO0 MEFs compared to WT MEFs, suggesting that the activation of PP1 contributes to the decrease in eIF2α phosphorylation in the 4-KO0 and 5-KO0 MEFs (FIG. 9H). Additionally, there was a trend for an increase in the phosphorylation of PP1α-³²⁰T in the 5-KO0 MEFs compared to the 4-KO0 MEFs (FIG. 9H). Since the reduced activity of PP1 in the 5-KO0 MEFs as compared to that in the 4-KO0 MEFs would enhance eIF2α phosphorylation in the 5-KO0 MEFs, the significant decrease in the phosphorylation of eIF2α-⁵¹S observed in the 5-KO0 MEFs as compared to the 4-KO0 MEFs, as a result of the loss of MARK2, supports the notion that MARK2 plays a major role in directly promoting eIF2α phosphorylation.

1.2.4 A PKCδ-MARK2-eIF2α signaling pathway in response to protein misfolding stress

To understand the regulation of MARK2 activation under conditions of protein misfolding, we sought to identify the upstream kinase that is responsible for activating MARK2 in response to the stress conditions. In the course of our studies, we tested PKCδ, a member of the PKC family, as a potential kinase of MARK2, because PKCδ was shown to be activated during stress responses, and another member of the PKC family was previously reported to phosphorylate MARK2 in the regulation of cell polarity. Suzuki et al., 2004; Hurov et al, 2004.

First, we generated MEF cell lines that stably expressed PKCδ and observed a substantial increase in the level of phosphorylated MARK2-⁵⁹⁵T, suggestive of the activation of MARK2 when the PKCδ level was elevated (FIG. 2D, left). The phosphorylation of eIF2α-⁵¹S was increased in correlation with that of MARK2-⁵⁹⁵T. Conversely, in PKCδ-knockout MEFs, the signals for phosphorylated MARK2, and accordingly that of eIF2α, were abolished (FIG. 2D, right). As controls, the total levels of MARK2 and eIF2α protein were not altered.

Next, we asked whether PKCδ would exhibit any direct kinase activity toward MARK2. Using in vitro kinase assays with purified proteins, we found that PKCδ significantly increased the level of phosphorylated MARK2, despite a background of autophosphorylation of MARK2, indicating that PKCδ has intrinsic kinase activity for MARK2 (FIG. 2E). In comparison, PKCδ did not show any kinase activity toward eIF2α in the in vitro kinase assay, indicating that PKCδ is not a direct kinase of eIF2α (FIG. 6B, lane 8). These results therefore point to a PKCδ-MARK2-eIF2α signaling pathway that operates in the cell. To determine whether PKCδ is required for the activation of MARK2-eIF2α signaling downstream, we applied MG132-induced proteotoxic stress to MEF cells with or without PKCδ. In the WT MEFs, MG132 elicited a robust up-regulation of phosphorylated MARK2-⁵⁹⁵T, reflective of increased MARK2 kinase activity; concomitantly, we observed a parallel increase in the phosphorylation of eIF2α-⁵¹S (FIG. 2F). In contrast, in the PKCδ-knockout MEFs, we saw a substantially diminished increase in the levels of phosphorylated MARK2-⁵⁹⁵T or eIF2α-⁵¹S in response to MG132, indicating inhibition of MARK2 kinase activity toward eIF2α (FIG. 2F). Furthermore, consistent with the activation of the MARK2-eIF2α pathway by MG132 treatment in the knockout MEFs lacking each of the 4 known kinases of eIF2α (PERK, HRI, PKR, and GCN2), PKCδ also was activated, as demonstrated by the phosphorylation of its threonine 505 (505T) site (FIG. 8C-FIG. 8F). Steinberg, 2004.

This result indicated that the PKCδ-MARK2-eIF2α signaling is independent of the previously known eIF2α kinases. Although the phosphorylation of eIF2α leads to attenuation of the translation of most transcripts, it also increases the translation of specific mRNAs such as activating transcription factor 4 (ATF4), Harding et al., 2000, as part of the stress response. Indeed, treatment of MEF cells with MG132, which induces proteotoxicity, increased the level of the ATF4 protein (FIG. 2G). The increase in the ATF4 level, however, was significantly reduced in knockout MEFs lacking PKCδ or MARK2 (FIG. 2G). Accordingly, the levels of eIF2α phosphorylation were significantly reduced in the absence of PKCδ or MARK2 (FIG. 2G). These observations demonstrate that PKCδ and MARK2 are major positive regulators of eIF2α phosphorylation during MG132-induced proteotoxic stress, providing evidence for a PKCδ-MARK2-eIF2α signaling cascade.

1.2.5 HSP90 Interacts with PKCδ and Mediates Stress-Dependent Activation of PKCδ

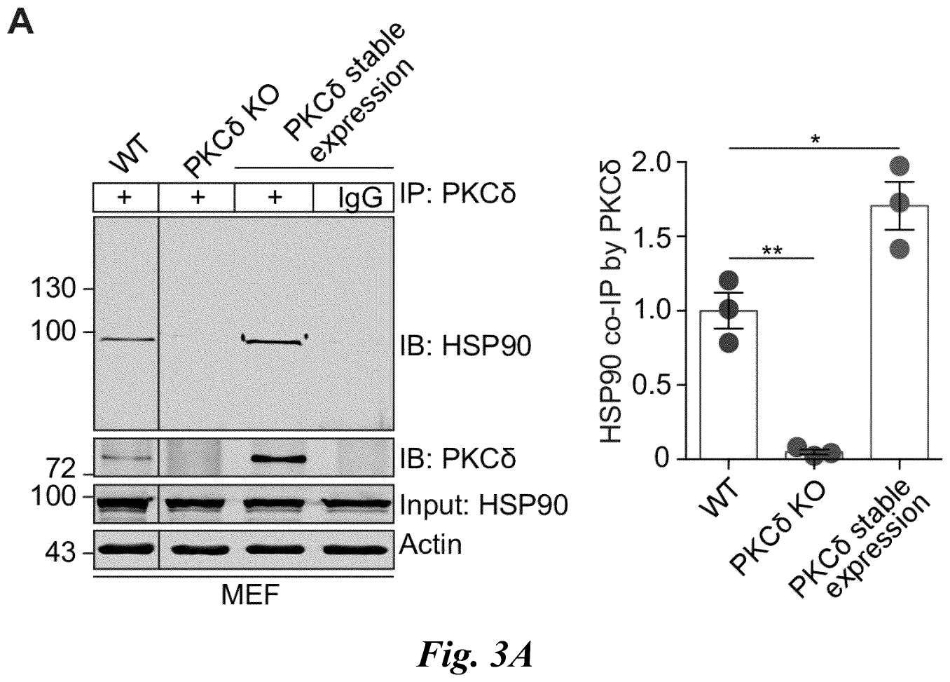

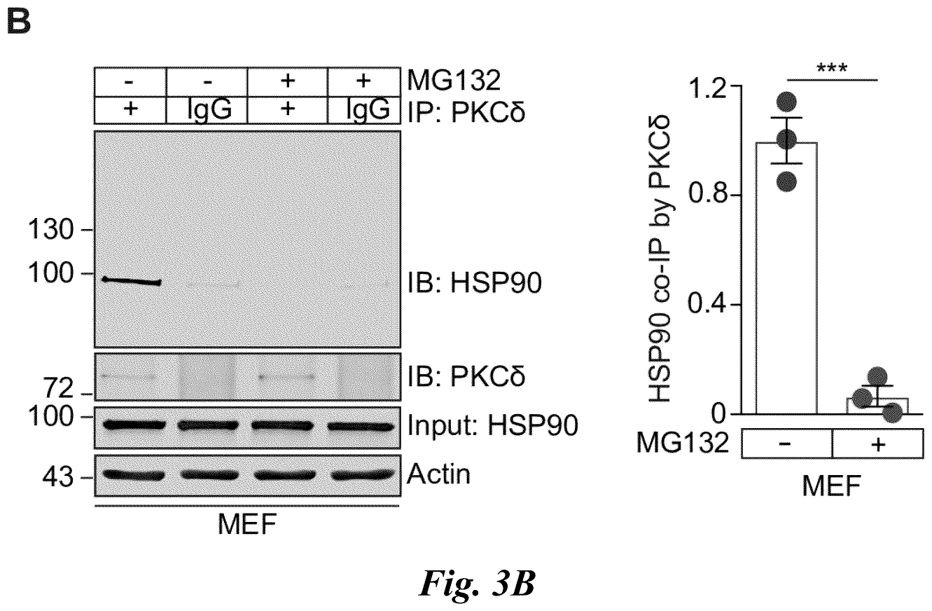

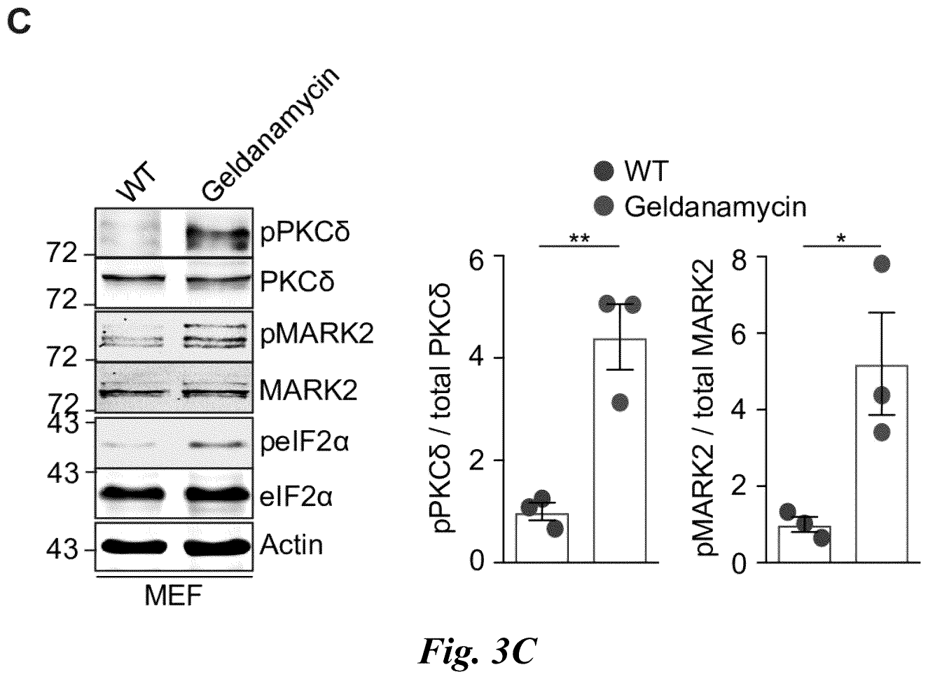

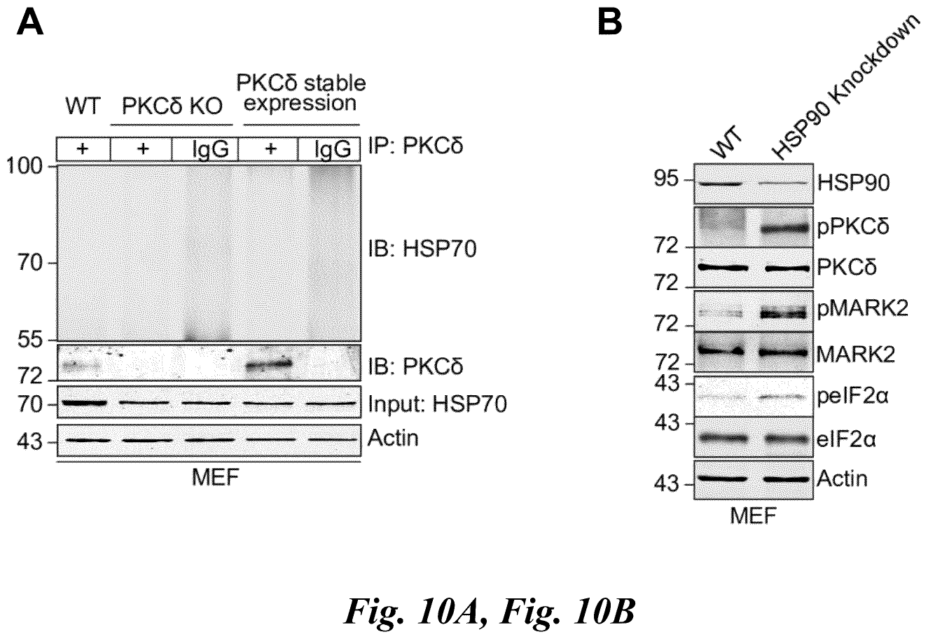

We next investigated how the PKC&-MARK2-eIF2α signaling pathway senses protein misfolding stress. Since misfolded proteins, including SOD1, are prone to interact with molecular chaperone proteins, Wang et al., 2009; Tummala et al., 2005, we reasoned that the upstream regulator PKCδ could potentially interact with a molecular chaperone and that disruption of this interaction by misfolded proteins during proteotoxic stress might be a mechanism to account for the activation of the PKCδ kinase. We searched a protein-protein interaction database, Szklarczyk et al., 2015, and identified several candidate interactors of PKCδ, including HSP90, HSP90a, and HSP70. To evaluate potential interactions between PKCδ and these molecular chaperones, we performed coimmunoprecipitation experiments with endogenous proteins in MEF cells. After immunoprecipitation of PKCδ, only HSP90 was pulled down by PKCδ (FIG. 3A). When the level of PKCδ protein was elevated via stable expression in MEF cells, more HSP90 protein could be coimmunoprecipitated by PKCδ. Conversely, depletion of PKCδ from MEFs or use of an IgG control instead of the anti-PKCδ antibody abolished the coimmunoprecipitation of HSP90, confirming the specific interaction between PKCδ and HSP90 (FIG. 3A). In comparison, we did not detect an interaction between PKCδ and HSP70 in the same immunoprecipitation assays using either WT MEFs or cells with stable expression or depletion of PKCδ (FIG. 10A). To test whether increased levels of misfolded proteins could disrupt the interaction between PKCδ and HSP90, we performed coimmunoprecipitation assays in MEF cells treated with MG132. We found that the MG132-induced proteotoxic stress completely abolished the interaction between PKCδ and HSP90, suggesting that the accumulated misfolded proteins compete for the binding of HSP90 and release PKCδ from the complex (FIG. 3B). To determine whether HSP90 regulates the activation of PKCδ, we tested the effect in MEF cells of a specific inhibitor of HSP90, geldanamycin, Whitesell et al., 1994, on the phosphorylation of PKCδ at residue threonine 505, which is required for the kinase activity of PKCδ. Steinberg, 2004.

We found that this HSP90 inhibitor substantially enhanced the phosphorylation of PKC-⁵⁰⁵T (FIG. 3C). Concomitant with the activation of PKCδ, there was an increase in the phosphorylation of MARK2-⁵⁹⁵T and eIF2α-⁵¹S in the cells treated with the HSP90 inhibitor (FIG. 3C). Similar results were observed for the increase in the phosphorylation of PKCδ-⁵⁰⁵T, MARK2-⁵⁹⁵T, and eIF2α-⁵¹S, when HSP90 was genetically knocked down in MEFs (FIG. 10B). These data demonstrate that inhibition of HSP90 results in the activation of the PKCδ-MARK2-eIF2α signaling pathway. Collectively, these results suggest that PKCδ and HSP90 form a complex that can sense protein misfolding stress through the intrinsic affinity of HSP90 for misfolded proteins and thereby regulate the activation of PKCδ-MARK2-eIF2α signaling.

### 1.2.6 Misfolded SOD1 Induces PKCδ-MARK2-eIF2α Signaling

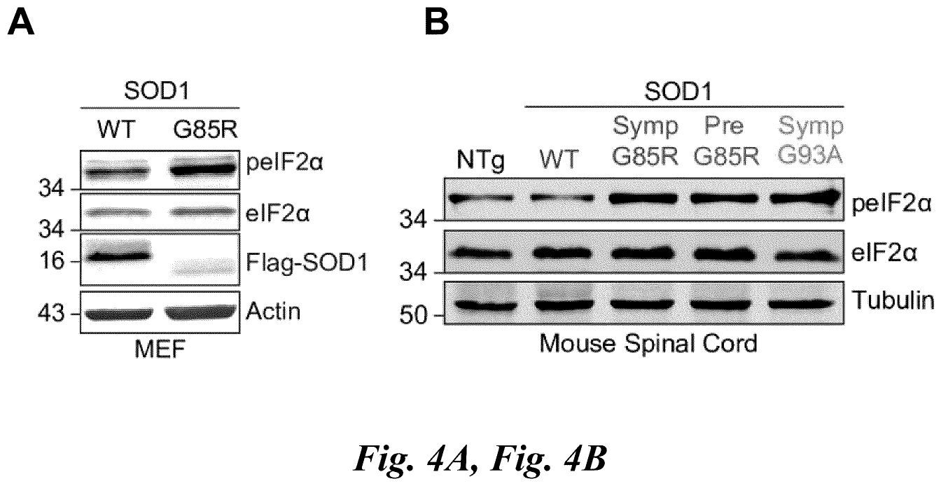

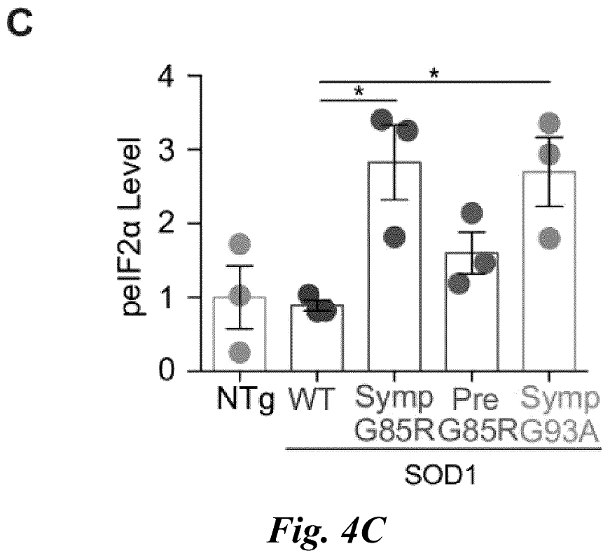

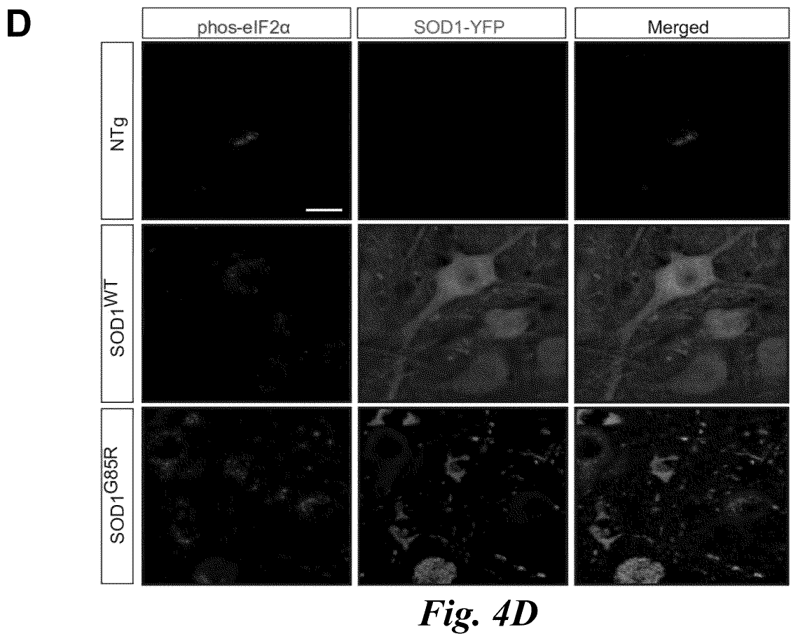

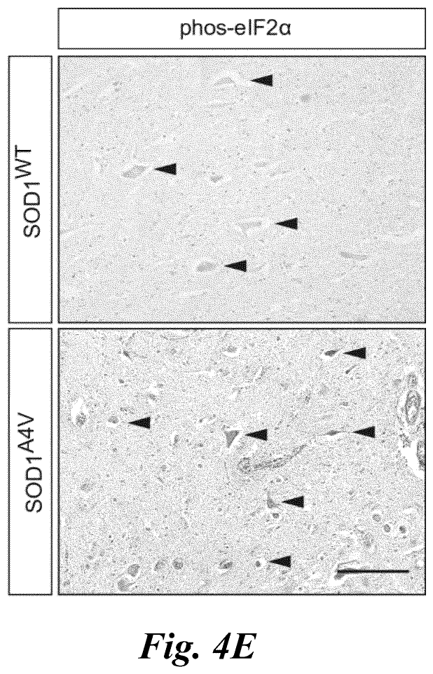

ALS-linked mutant SOD1 proteins, including the G85R variant, are prone to misfolding and aggregation, providing a sensitive molecular model for studying proteotoxicity. Wang et al., 2009; Periz et al., 2015. Next, we asked whether the misfolded mutant SOD1^{G85R} affects the phosphorylation of eIF2α. We generated stable MEF lines that express SOD1^{WT} or SOD1G^{85R} in an inducible manner. Upon induction, we found that SOD1^{G85R} caused a marked increase in the phosphorylation of eIF2α-⁵¹S when compared to the SOD1^{WT} control (FIG. 4A). We then used a set of transgenic mouse models expressing WT or disease-associated SOD1 variants in order to investigate the regulation of eIF2α phosphorylation. We have previously shown that SOD1^{G85R-YFP} causes age-dependent motor neuron degeneration in mice, with a marked accumulation of protein aggregates upon disease onset; in contrast, control SOD1^{WT-YFP} mice are free of disease symptoms and protein aggregation pathology. Wang et al., 2009.

When compared to nontransgenic or SOD1^{WT-YFP} controls, SOD1^{G85R-YFP} mice at the presymptomatic stage showed a moderate increase in eIF2α-⁵¹S phosphorylation in the spinal cords, as measured by immunoblotting; however, in the symptomatic SOD1^{G85R-YFP} mice, the phosphorylation of eIF2α-⁵¹S was remarkably increased (FIG. 4B and FIG. 4C). The increase in eIF2α-⁵¹S phosphorylation in SOD1^{G85R-YFP} motor neurons, which harbor pronounced cytoplasmic aggregates in affected mice, was confirmed by immunofluorescent staining of spinal cord sections (FIG. 4D). A similar increase in eIF2α-⁵¹S phosphorylation was observed and quantified by immunoblotting in transgenic mice expressing another disease-associated mutant, SOD1^{G93A} at the symptomatic stage (FIG. 4B and FIG. 4C). Furthermore, in a human ALS patient carrying the SOD1^{A4}v mutation, we found by immunohistochemical analysis that the eIF2α phosphorylation also was markedly increased in spinal cord motor neurons (FIG. 4E). These results suggest that eIF2α phosphorylation and subsequent translational regulation are a pathological consequence of mutant SOD1 in mammalian systems.

1.2.7 Increased Phosphorylation of PKCδ-⁵⁰⁵T and MARK2-⁵⁹⁵T in ALS Mice and Patients

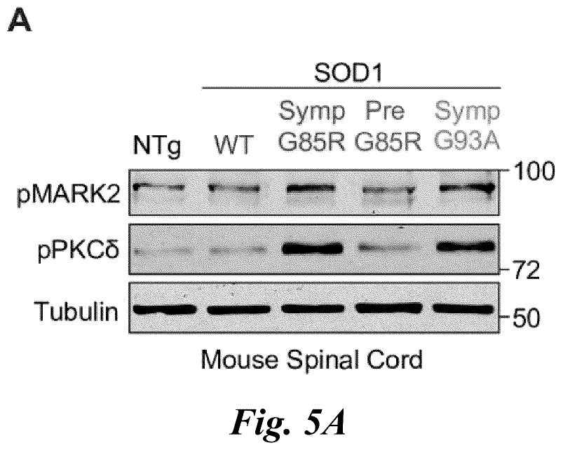

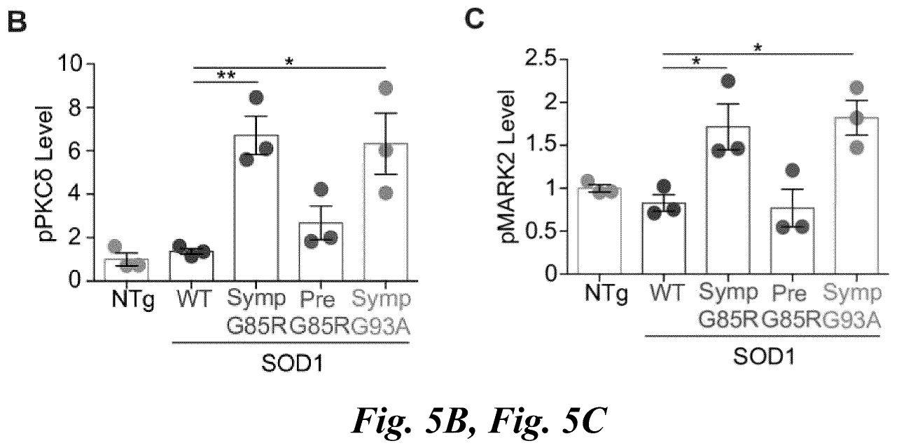

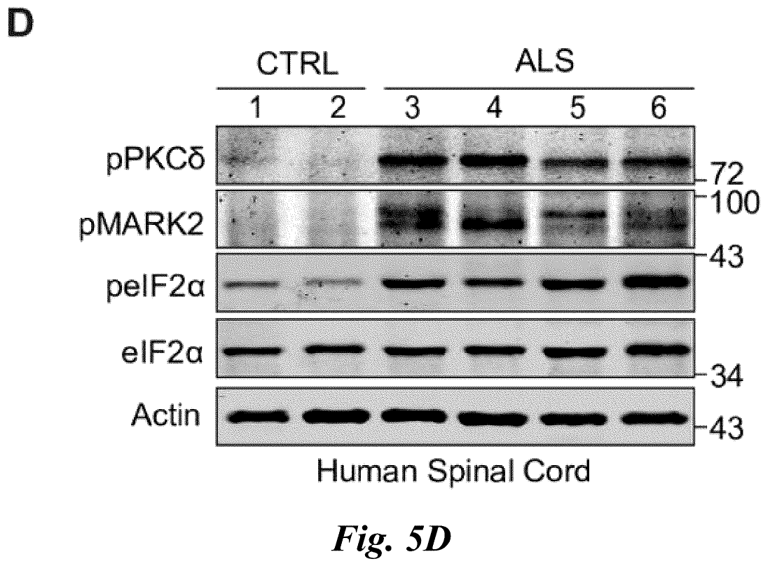

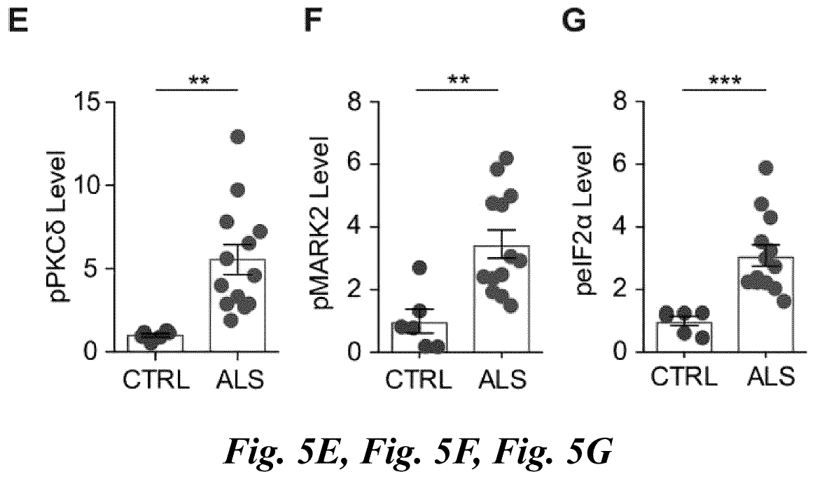

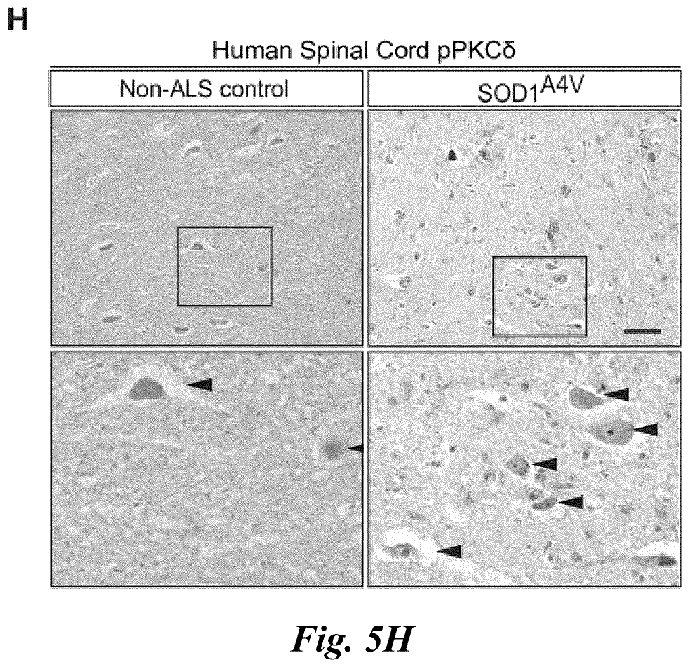

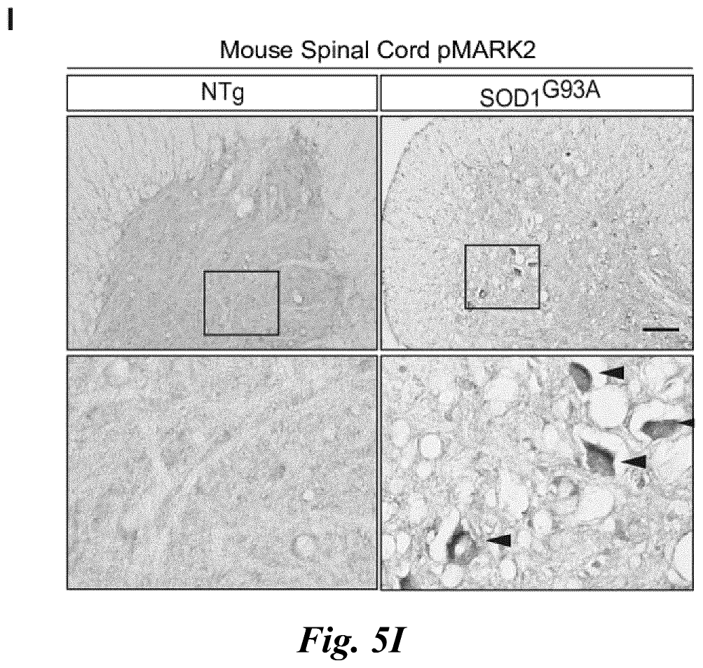

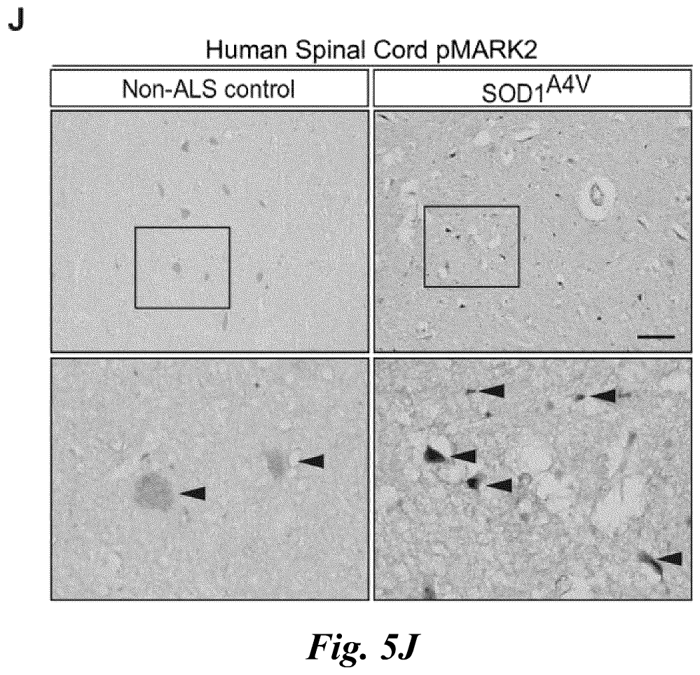

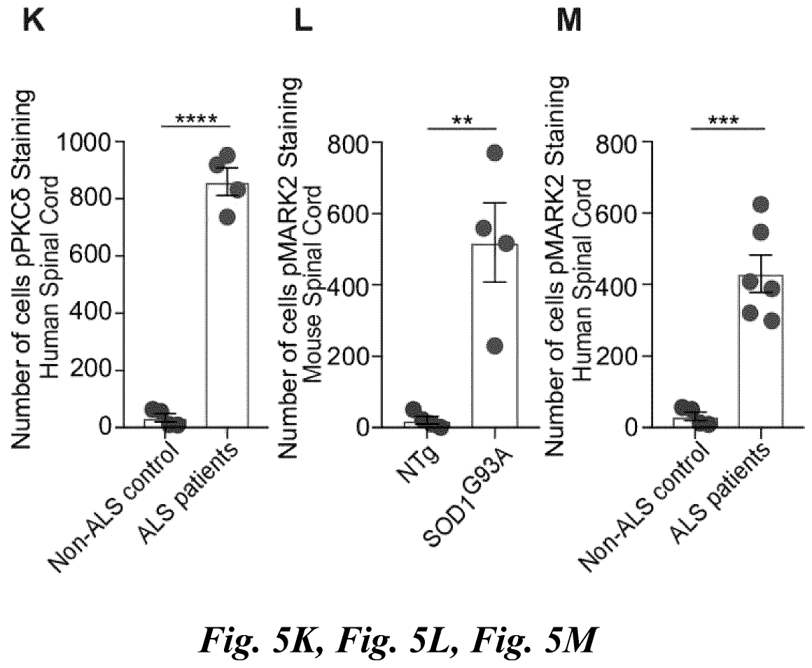

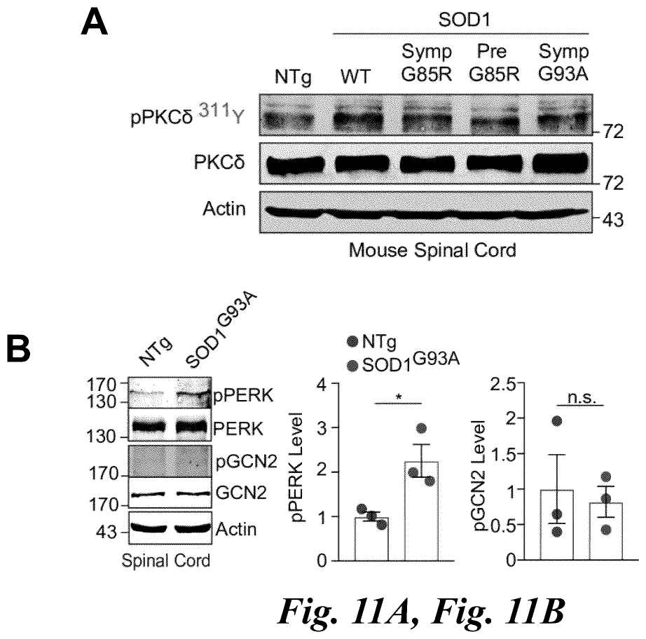

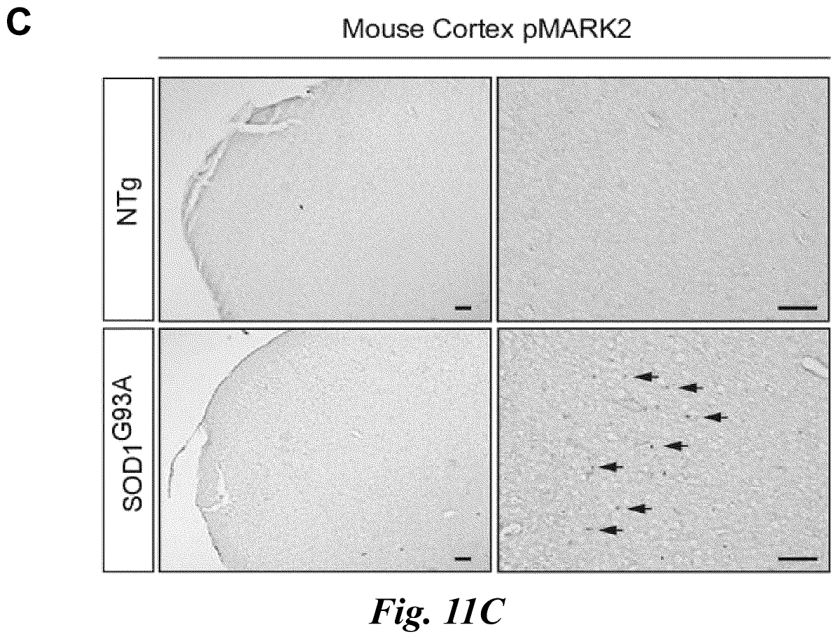

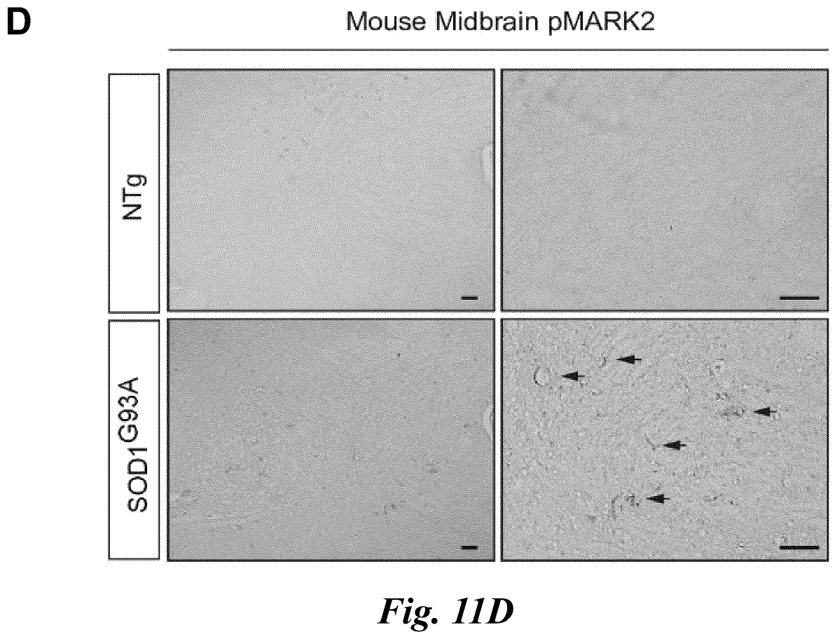

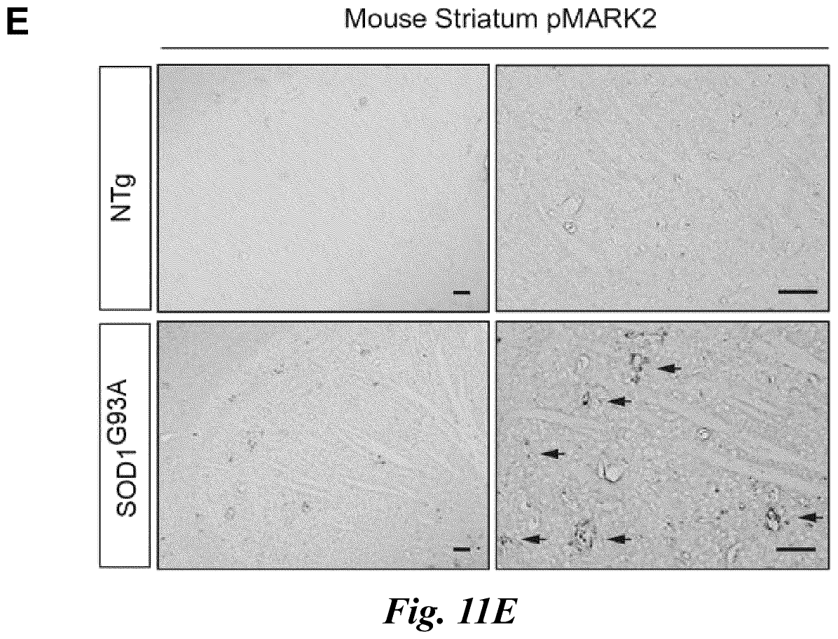

Next, we examined the status of the PKCδ-MARK2-eIF2α signaling pathway in ALS mouse models and patients. To address whether PKCδ is activated by misfolded SOD1 proteins, and since the phosphorylation of PKCδ-⁵⁰⁵T is necessary for its kinase activity, Steinberg, 2004, we analyzed the phosphorylation of PKCδ-⁵⁰⁵T in the spinal cords of transgenic mice expressing various ALS-linked SOD1 mutants. When compared to age-matched nontransgenic and SOD1^{WT-YFP} transgenic mice, SOD1^{G85R-YFP} mice at the symptomatic stage exhibited a substantial increase in the phosphorylation of PKCδ-⁵⁰⁵T, as shown by immunoblotting of spinal cord tissues (FIG. 5A). Presymptomatic SOD1^{G85R-YFP} mice did not show such changes in the phosphorylation of PKCδ, whereas in transgenic mice expressing another disease mutant, SOD1^{G93A} similar levels of up-regulation of PKCδ phosphorylation were seen at the symptomatic stage (FIG. 5A). As a control, we asked whether phosphorylation of PKCδ at another known site, tyrosine 311 (311Y), a docking site unrelated to kinase activity but used for signal-regulated scaffolding, Steinberg, 2004, is affected by mutant SOD1. In the spinal cords of symptomatic mutant SOD1 transgenic mice, immunoblots showed no change in the levels of phosphorylated PKCδ-311Y as compared to SOD1^{WT} or nontransgenic controls (FIG. 11A). Furthermore, the changes in the levels of phosphorylated MARK2-⁵⁹⁵T, which indicate MARK2 activity toward eIF2α, were in complete accordance with the activation of PKCδ in the mouse spinal cords (FIG. 5A-FIG. 5C). Additionally, consistent with the previous reports of ER stress in the SOD1 mouse models of ALS, Nishitoh et al., 2008; Kikuchi et al, 2006, the phosphorylation of PERK, but not that of GCN2, was increased in the spinal cord tissues from symptomatic mutant SOD1 mice (FIG. 11B), suggesting that both PERK and MARK2 could contribute to the phosphorylation of eIF2α in these mice. Together, these results suggest that the PKCδ-MARK2-eIF2α signaling is a previously unrecognized pathway implicated in the neurodegenerative mouse models associated with misfolded SOD1 proteins.

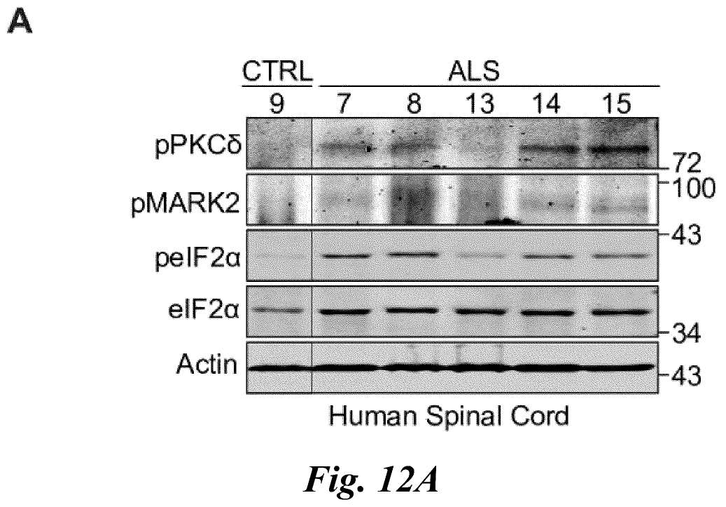

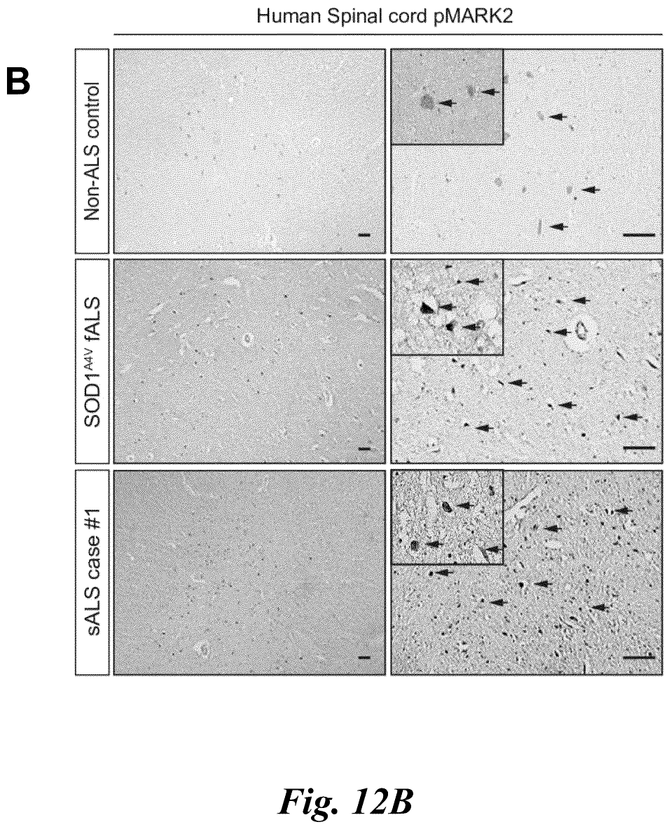

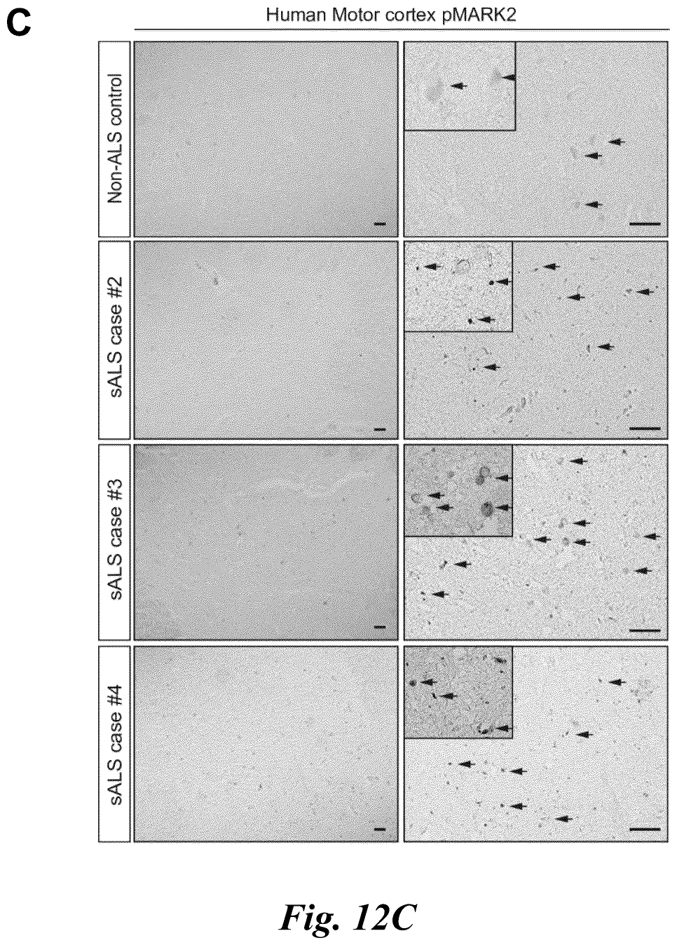

To extend our findings to human patients, we performed immunoblot analysis of PKCδ and MARK2 in spinal cord tissues from ALS patients and non-ALS controls. Of the 13 ALS patients' spinal cords examined, most showed a substantial increase in the phosphorylation of PKCδ-⁵⁰⁵T when compared to non-ALS controls (FIG. 5D and FIG. 5E, FIG. 12A), suggesting that activation of PKCδ in the spinal cord is a general feature in ALS patients. In the patients' tissues that showed up-regulated phosphorylation of PKCδ-⁵⁰⁵T, the phosphorylation of MARK2-⁵⁹⁵T also was significantly increased (FIG. 5D and FIG. 5F, and FIG. 12A). The increases in the phosphorylation of PKCδ and MARK2 were correlated with the increase in the phosphorylation of eIF2α-⁵¹S in the patients' spinal cords (FIG. 5D and FIG. 5G, and FIG. 12A). Furthermore, we performed immunohistochemical staining of the spinal cords from ALS patients including one carrying the mutation SOD1^{A4V} using antibodies against phosphorylated PKCδ-⁵⁰⁵T and confirmed that the PKCδ phosphorylation was markedly increased in the affected spinal cords (FIG. 5H and FIG. 5K). These results demonstrate that the increase in the catalytically active form of PKCδ, as marked by phosphorylated PKCδ-⁵⁰⁵T, is a pathological feature of neurodegeneration in ALS patients.

We also investigated the neuropathology of MARK2 in ALS patients and animal models. In nontransgenic control mice, immunostaining with an antibody specific for MARK2-⁵⁹⁵T showed a diffuse pattern in the gray matter of the anterior horn of the spinal cord. However, in age-matched symptomatic SOD1^{G93A} mice, the staining was markedly increased in both the gray and white matter (FIG. 5I and FIG. 5L). A subset of neurons had intense staining in the soma. A similar increase in the staining of phosphorylated MARK2-⁵⁹⁵T was observed in the brain cortex, striatum, and midbrain of the SOD1^{G93A} mice (FIG. 11C-FIG. 11E). Furthermore, we studied the pattern of phosphorylated MARK2 in human spinal cord tissues. In heathy control cases, the immunohistochemical staining of phosphorylated MARK2 was relatively light, except for a set of well-demarcated large neurons. In contrast, in the familial ALS patient with the SOD1^{A4V} mutation, the signals for phosphorylated MARK2 in the cells were increased in both number and intensity (FIG. 5J and FIG. 5M). Moreover, in a set of four sporadic ALS patients that were examined, a similar increase in the staining of phosphorylated MARK2-⁵⁹⁵T also was observed in both the spinal cord and motor cortex (FIG. 12B and FIG. 12C). These results suggest that the activation of MARK2 is a notable pathologic feature in the spinal cords of ALS patients who suffer from the motor neuron degeneration.

### 1.3 Discussion

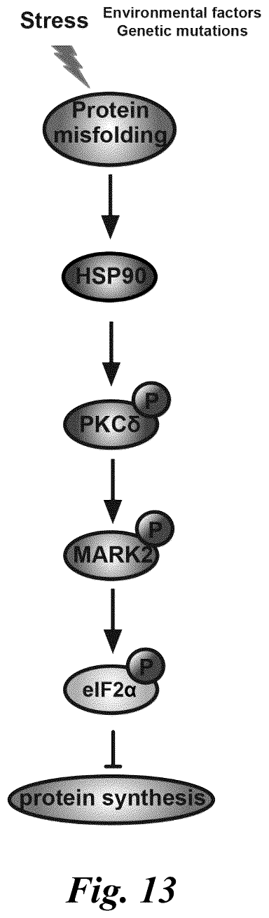

In the present study, we describe a previously unrecognized signaling pathway whereby the proteotoxic stress regulates translation through eIF2α phosphorylation (FIG. 13). We identified MARK2 as a direct kinase for eIF2α in this stress-response signaling. PKCδ acts upstream of MARK2 and senses the protein misfolding stress through its interaction with the molecular chaperone HSP90. The identification of this pathway unveils a distinct stress response mechanism that is important for cytosolic protein homeostasis. Furthermore, the activation of the PKCδ-MARK2-eIF2α pathway in ALS mouse models and human patients associated with protein misfolding suggests that this stress signaling may be important for the pathogenesis of relevant neurodegenerative diseases. The rate-limiting step in protein synthesis in eukaryotes is at the level of translation initiation. Holcik and Sonenberg, 2005.

One of the key regulatory factors for translation initiation, eIF2α, is phosphorylated at the conserved residue serine 51 by four previously known kinases, including PKR, PERK, HRI, and GCN2, which mediate different stress signals in an integrated stress response network. Although it was previously suggested that there are only four eIF2α kinases, Taniuchi et al., 2016, we provide both in vitro and in vivo evidence, including results from knockout cells lacking the four known eIF2α kinases, that MARK2 is a previously unrecognized kinase for eIF2α and that it plays an important role in mediating the phosphorylation of eIF2α upon proteotoxic stress. The four previously known eIF2α kinases are closely related phylogenetically and their kinase domains are similar structurally, while MARK2 is phylogenetically distinct, Manning et al., 2002, thus it would be interesting to investigate the structural basis for MARK2 to recognize eIF2α in future studies.

The observation that cells lacking all 5 eIF2α kinases, including MARK2, were still capable of exhibiting a trend for enhanced phosphorylation of eIF2α, albeit to a diminished degree, in response to stress suggests that there may be other factors influencing eIF2α phosphorylation or dephosphorylation. Together, these results expand our understanding of the pathways in the integrated stress response and reveals MARK2 as a distinct signaling hub for the regulation of translation.

In the present study, we also identified PKCδ as an upstream kinase that promotes both basal and induced phosphorylation of eIF2α. PKCδ does not directly phosphorylate eIF2α, but instead acts as a direct kinase of MARK2. It is a multifunctional kinase that influences several cellular processes, including growth, differentiation, and apoptosis. Kikkawa et al., 2002; Jackson and Foster, 2004. The PKCδ-MARK2-eIF2α pathway identified in this study demonstrates a role for PKCδ in the fundamental cellular regulation of translational control. Our identification of a stress-dependent interaction between PKCδ and HSP90 suggests that PKCδ can sense changes in the levels of misfolded proteins through its competitive binding to HSP90. This scenario is reminiscent of the mechanism underlying the activation of HSF1 by unfolded proteins through a dynamic interaction between HSF1 and HSP90, Zou et al., 1998, or the activation of PERK by unfolded proteins in the ER lumen through a dynamic interaction between PERK and the ER chaperone protein BiP (immunoglobulin heavy-chain-binding protein). Bertolotti et al., 2000.

We have demonstrated that PKCδ-MARK2-eIF2α signaling is activated by protein misfolding stress, independently of PERK. Thus, the identification of the PKCδ-MARK2-eIF2α pathway provides a mechanism for direct signal transduction from cytosolic protein misfolding to translational control. Most neurodegenerative diseases are associated with toxicities resulting from the accumulation of misfolded proteins, but the molecular and cellular consequences of the protein misfolding stress have not been fully determined. The activation of the PKCδ-MARK2-eIF2α pathway seen in the ALS models and patients' tissues examined in the present study suggests that translational regulation is one of the pathological consequences of the disease. The translational attenuation as a result of the activated PKCδ-MARK2-eIF2α pathway may first serve as an adaptive stress response that lowers the protein burden during proteotoxic stress. A prolonged activation of the pathway under chronic stress, however, could induce built-in mechanisms of cell death. Basu and Pal, 2010; Rutkowski and Kaufman, 2007.

In agreement with the importance of translational regulation for neuronal health, increased eIF2α phosphorylation is a common pathological hallmark of major neurodegenerative diseases. Moreno et al., 2012; Chang et al., 2002; Ryu et al., 2002. Moreover, modulation of eIF2α phosphorylation have been shown to affect the phenotypes of animal models of neurodegeneration with different outcomes of alleviation or aggravation of disease phenotypes associated. Ma et al., 2013; Kim et al., 2014; Wang et al., 2014.

In sum, our results in the present study provide a previously unrealized mechanism for regulating translation during stress and neurodegenerative conditions. Further studies of this signaling pathway may expand our understanding of the regulation of protein homeostasis and its role in the development of relevant human diseases.

### 1.4 Methods

1.4.1 DNA constructs

For mammalian expression, human SOD1^{WT} and SOD1^{G85R} were subcloned into the pEFBOS plasmid as previously described. Periz et al., 2015. The pEGFP-C3 expression plasmid was obtained from Addgene (6082-1). The human MARK2 expression plasmid (HsCD00074644) was obtained from the DNASU repository. Both eIF2α and MARK2 were subcloned into the pDEST plasmid using the Gateway system (Thermo Fisher, United States of America). Kinasedead MARK2^{KD} mutant was generated by PCR, amplifying the fragment without the kinase domain. eIF2α^{S51A} and eIF2α^{S51D} mutants were constructed using the Q5 Site-Directed Mutagenesis Kit (New England Biolab E0554). For the NanoBRET assay, eIF2α^{WT}, eIF2α^{S51A}, or eIF2α^{S51D} was subcloned into the pHTN HaloTag CMV-neo Vector (Promega JF920304). MARK2 or PERK was subcloned into the pNLF1-C [CMV/Hygro] Vector (Promega KF811458). The HaloTag and NanoLuc expression vectors as well as the positive control of p53 and MDM2 fusion expression vectors are included in the NanoBRET PPI Systems Kit (Promega, USA).

### 1.4.2 Genome Editing Using the CRISPR-Cas9 System

The specific gRNA sequences were selected by using the CRISPR design tool from Benchling. The gRNAs were cloned into the gRNA/Cas9 expression vector pLenti-CRISPR v2, conferring resistance to puromycin (Addgene 52961) or blasticidin (Addgene 98293), or the gRNA multiplexing system STAgR. Breunig et al., 2018. After cell transduction with the lentiviruses expressing the Cas9/gRNAs, single cell colonies were isolated based on puromycin or blasticidin resistance. The resulting cell lines were verified for their genotypes by sequencing the targeted locus or probing the targeted protein through immunoblot analysis.

### 1.4.3 Cell Lines

MEFs were grown in Dulbecco's modified Eagle's medium (DMEM) supplemented with 10% fetal bovine serum (FBS) and antibiotic-antimycotic solution at 37° C. with 5% CO₂. The MEFs include knockout lines lacking PERK, Harding et al., 2000, GCN2, Zhang et al., 2002, HRI, McEwen et al., 2005, PKCδ, Humphries et al., 2006, MARK2, Hurov et al., 2001, or PKR, Yang et al., 1995, and a knock-in line eIF2α^{S51A} MEFs. Scheuner et al., 2001. MARK2 stable cell lines were generated by transfecting WT MEF cells with the MARK2^{WT} expression construct (DNASU HsCD00074644) or the MARK2^{T595A} mutant version and then passaged into selective medium containing 3 μg/mL puromycin. Human SOD1^{WT} and SOD1^{G85R} stable cell lines were generated by transfecting WT MEF cells with the CMV.TO-3XnFlag-SOD1^{WT}-pkg-tetR-Puro or CMV. TO-3XnFlag-SOD1^{G85R}-pkg-tetR-Puro vector and then selecting with 3 g/mL puromycin. To generate the MEF lines lacking multiple eIF2α kinases, the CRISPR-Cas9 system was used to knock out PEKR, GCN2, HRI, and MARK2 in the existing PKR knockout MEF line. Yang et al., 1995.

A remnant C-terminal fragment of PKR in the knockout MEF line was further deleted using the CRISPR-Cas9 system. The detection of the C-terminal fragment of PKR was achieved by treating cells with mIFN-α (mouse interferon-α, Biolegend 752804, USA) to induce PKR expression followed by immunoblotting with an antibody against PKR (Santa Cruz SC-6282, USA). Baltzis et al., 2002.

The production of lentiviruses and cell transduction were performed using a previously described protocol with modifications. Kutner et al., 2009. Single cell colonies that survived puromycin selection were individually expanded in the selective medium to establish independent lines. HSP90 knockdown in MEFs was achieved by infecting cells with virus derived from pLenti-CRISPR v2 harboring the HSP90-specific gRNA (50-ACCCCAGTAAACTGGACTCG-30), and a population of puromycin-selected cells were used. Human MARK2 knockout cells were generated using CRISPR-Cas9 editing in a haploid human HAP1 cell line (HZGHC000328c013) (Horizon Discovery, United Kingdom). HAP1 cell lines were cultured in Iscove's modified Dulbecco's medium (IMDM) with 10% FBS. HEK293 cells were grown in DMEM with 10% FBS.

### 1.4.4 Immunoblotting

Cells were washed twice with 1× PBS and then lysed and harvested on ice in RIPA solution (50 mM Tris-HCl (pH 7.6); 150 mM NaCl; 1% NP-40; 1% SDS; 100 mM sodium fluoride; 17.5 mM β-glycerophosphate; 0.5% sodium deoxycholate; 10% glycerol). The RIPA buffer was supplemented with EDTA-free protease inhibitor cocktail (Roche, USA), phosphatase inhibitor cocktail 2 and phosphatase inhibitor cocktail 3 (Sigma-Aldrich, USA), 1 μM phenylmethanesulfonyl fluoride, and 2 μM sodium orthovanadate. Lysates were kept cold on ice, pulse-sonicated for 10 min, and then centrifuged at 12,000 g at 4° C. for 10 min. The protein content of each sample was determined by a bicinchoninic acid (BCA) assay (Thermo Fisher). Equal amounts of total protein extract were resolved by SDS-PAGE and transferred to nitrocellulose membranes (Millipore HATF08550, USA). The blots were blocked with 5% w/v BSA and 0.05% NaN₃ in TBST and incubated with primary antibodies at 4° C. overnight, then finally incubated with appropriate secondary antibodies. The antibodies used include those against eIF2α (Cell Signaling 5324, USA), peIF2α (Cell Signaling 9721), PERK (Cell Signaling 3192), pPERK-⁹⁸⁰T (Cell Signaling 3179), PKR (Santa Cruz SC-708 and SC-6282), HRI (Millipore 07-728), GCN2 (Cell Signaling 3302), pGCN2-899T (Abcam ab75836, USA), ATF4 (Cell Signaling 11815), HSP90 (Cell Signaling 8165), HSP70 (Cell Signaling 4872), Flag (Sigma F1804), Actin (Santa Cruz SC-47778), Tubulin (Proteintech 10068-1-AP, USA), PKCδ (Santa Cruz SC-937; Cell Signaling 2058), pPKCδ-311Y (Cell Signaling 2055), pPKCδ-⁵⁰⁵T (Cell Signaling 9374), MARK2 (Abcam ab135816; Santa Cruz SC-365405), pMARK2-⁵⁹⁵T (Abcam ab34751), PP1a (Cell Signaling 2582), pPP1α-³²⁰T (Cell Signaling 2581), and puromycin (Millipore MABE343). Images were captured with an Odyssey imager and analyzed with Image Studio software (Licor 9120, USA).

### 1.4.5 In Vivo Labeling for Protein Synthesis Analysis

Stable MEF cell lines overexpressing MARK2^{WT} or MARK2^{T595A} were plated onto 6-well plates (2×10⁵ cells per well) overnight. For puromycin labeling, cells were treated with 10 μg/mL puromycin in culture medium for 10 min and then washed 3 times with 1× PBS and lysed with RIPA buffer as described above. The cell lysate was analyzed by immunoblotting against puromycin. For ³⁵S labeling, cells were incubated with methionine- and cysteine-free DMEM supplemented with 10% FBS (MilliporeSigma F0392) for 1 h. A total of 200 μCi of [³⁵S]-methionine and [³⁵S]-cysteine (PerkinElmer NEG772002MC, USA) was then added to each dish to metabolically label the cells for 1 h. After radiolabeling, the cells were washed 3 times with 1× PBS and lysed with RIPA buffer as described above. A total of 20 ng of cell lysate was added into 4 mL of liquid scintillation cocktail (MP Biomedicals 01882475-CF, USA), and the radioactivity was detected using LS6500 Liquid Scintillation Counter (Beckman Coulter, USA).

### 1.4.6 Immunoprecipitation

Cells were washed twice with 1× PBS and then lysed on ice in lysis buffer (50 mM Tris-HCl (pH 7.5); 150 mM NaCl; 1% NP-40; 1 mM EDTA; 0.5% sodium deoxycholate). The lysis buffer was supplemented with EDTA-free protease inhibitor cocktail (Roche). The cell lysates were immunoprecipitated with anti-PKCδ antibody (Cell Signaling 2058) using protein A/G magnetic beads. The beads were washed 3 times with washing buffer (50 mM Tris-HCl (pH 7.5); 150 mM NaCl; 1% NP-40; 1 mM EDTA) and then eluted with low-pH elution buffer at room temperature for 10 min. The eluents were neutralized with 1MTris-HCl (pH 8.0) and separated by SDS-PAGE and immunoblotted with antibodies against heat shock proteins including HSP90. For the coimmunoprecipitation analyses of heat shock proteins, MEF cells were heat shocked at 44° C. as previously described. Zou et al., 1998. Aliquots of the whole-cell lysates were immunoblotted using Actin and PKCδ antibodies.

### 1.4.7 Protein Purification

The protein kinases and substrates used in the in vitro kinase activity assays were expressed from bacterial, insect, or mammalian cells. Proteins purified using the E. coli strain Rosetta include MARK2^{WT}, MARK2^{T95A}, eIF2α^{WT}, and eIF2α^{S51A}. The cDNAs encoding these proteins were cloned into the pET28a plasmid with His tags, and the protein expression was induced by IPTG. E. coli cells were grown until the OD600 reached 0.4 to 0.6 before induction with 0.1 mM IPTG at 16° C. for 24 h. E. coli cells were harvested and suspended using lysis buffer (50 mM NaH₂PO₄; 300 mM NaCl; 10 mM imidazole; 0.05% Tween 20 (pH 8.0); and EDTA-free protease inhibitor cocktail [Roche]). Cells were kept cold on ice, lysed with a French pressure cell for 10 to 15 min, and then centrifuged at 10,000 g at 4° C. for 30 min. The lysates were immunoprecipitated using Ni-NTA agarose (Qiagen 30210, USA) at 4° C. for 1 h. The Ni-NTA agarose was washed with washing buffer (50 mM NaH2PO4; 300 mM NaCl; 20 mM imidazole; 0.05% Tween 20 (pH 8.0)) twice and the protein eluted by using elution buffer (50 mM NaH₂PO₄; 300 mM NaCl; 250 mM imidazole; 0.05% Tween 20 (pH 8.0)). The eluted proteins were passed through molecular weight cut-off centrifugal filters (Millipore) to remove imidazole and stored in buffer (20 mM Tris-HCl; 150 mM NaCl; 0.1 mM DTT) at −80° C. In addition, recombinant proteins expressed in Sf9 insect cells after infection with recombinant baculovirus include GST-tagged PKR, PKCδ, TTK, BMPR1A, and MARK2 and His-tagged PYK2 and eIF2α. These proteins were purified using a standard protocol with affinity column chromatography on glutathione columns by SignalChem (Canada). The purified proteins were diluted in a kinase buffer with 0.05 nM DTT. MBP proteins that were used as the universal kinase substrate were obtained from SignalChem (M42-51N). For mammalian expression of recombinant proteins, MARK2WT, MARK2^{T595}A, and a GFP control were expressed and purified from HEK293 cells. The cDNAs were cloned into a modified pCDNA3.1 plasmid to express Flag-tagged proteins in a tet-inducible manner as described previously. Periz et al., 2015.

The constructs were transfected into HEK293 cells, which were treated with 0.5 ug/mL doxycycline to induce expression. The cells were lysed in RIPA buffer (50 mM Tris-HCl; 150 mM NaCl; 1% NP-40; 1 mM PMSF (pH 8.0); and EDTA-free protease inhibitor cocktail [Roche]). The cell lysates were incubated with anti-Flag M2 magnetic beads (SIGMA M8823) for 24 h at 4° C. The beads were then washed with washing buffer (50 mM Tris-HCl (pH 7.5); 150 mM NaCl) several times. The proteins were eluted from the beads by adding 5 volumes of 5 μg/L 3xFlag peptide solution, followed by incubation at 4° C. for 1 h.

### 1.4.8 In Vitro Kinase Activity and Kinetics Assays

For the in vitro kinase activity assay based on radiolabeling and gel electrophoresis, the reaction mix included a kinase protein at 0.04 μg/μL (MARK2 and MARK2^{T595A} at 0.51 μM, PKR at 0.54 M, PKCδ at 0.51 μM, and the control GFP at 1.48 M) and a substrate protein at 0.2 μg/μL (MBP at 9.3 μM, eIF2α at 5.26 μM, and MARK2 at 2.56 μM), 50 μM cold ATP, and [γ-³²P]-ATP (1 mCi/100 μL, PerkinElmer) diluted 1:300 in the kinase assay buffer (Signal-Chem, KO1-09). The reactions were incubated at 30° C. for 15 min before being analyzed by SDS-PAGE. Radioactive signals were detected with a FLA7000 imager (Fujifilm FLA7000, USA). For the in vitro kinase kinetic analysis, the Kinase-Glo assay was used to measure kinase activities by quantifying ATP consumption via luminescent signals (Promega V6711). In the initial round of analysis, the kinase proteins were serially diluted as indicated, while the substrate protein MBP was kept constant at 0.1 μg/μL (4.65 μM) with 5 μM of ATP supplemented. In the subsequent round of analysis, the Km concentrations of PKR and MARK2 as determined above were used, while eIF2α as the substrate was serially diluted as indicated, with 5 μM of ATP supplemented. The MARK^{KD} mutant was used at the same concentrations as those of its WT counterpart. The reactions were incubated at 30° C. for 60 min before addition of 1:1 volume of the Kinase-Glo reagent (Promega), followed by incubation at room temperature for 10 min. The Luminescence was detected with the Synergy H1 microplate reader (Bio-Tek, USA).

### 1.4.9 NanoLuc-Based Bioluminescence Resonance Energy Transfer Assays

The NanoBRET assays were performed according to the manufacturer's protocol (Promega NanoBRET Protein:Protein Interaction System), with some modifications. For each individual population, cells were seeded at 2×10⁵ cells/mL into 96-well plates (Corning Costar 3917 white opaque assay plates) and incubated in DMEM supplemented with 10% FBS and antibiotic-antimycotic solution at 37° C. with 5% CO₂ for 24 h. After 24 h, the cells were cotransfected with a combination of a NanoLuc fusion protein vector and a HaloTag fusion protein vector using jetPRIME Transfection Reagent and incubated at 37° C., 5% CO₂ for 16 to 24 h. After 24 h, NanoBRET Nano-Glo Substrate (Promega) was added to the transfected cells, and the fluorescence signal was measured at 460 nm and 618 nm within 10 min of substrate addition. A Synergy H1 Hybrid Reader (BioTek) with a custom filter cube (450 nm/610 nm) was used to measure the luminescence values (6 mm read height, 1 s integration time, 100 to 160 gain value). Mean corrected milliBRET (mBU) values were calculated using equations from the manufacturer's protocol (Promega, NanoBRET PPI Systems, N1821).

### 1.4.10 Mouse and Human Tissues

The SOD1 transgenic mice used in this study have been previously characterized: the SOD1^{G93A} line [B6SJL-TgN (SOD1^{G93A})1Gur; Jackson Laboratory], Gurney et al., 1994, and the SOD1^{G85R-YFP} and SOD1^{WT-YFP} lines. Wang et al., 2009. Transgenic mice were identified by PCR amplification of DNA extracted from tail biopsies. Mice were euthanized in a CO₂ chamber, and fresh tissues were harvested by flash-freezing in liquid nitrogen and then stored at −80° C. For immunoblot analysis, spinal cords were rinsed with cold PBS and homogenized with cold RIPA buffer using glass tissue grinders. The homogenates were then centrifuged at 4° C. at 1,000 g for 10 min, and the supernatants were centrifuged again at 16,000 g for 10 min, with the final supernatant used for immunoblot analysis. The animal protocol (MO18H105) was approved by the Animal Care and Use Committee of the Johns Hopkins Medical Institutions. Human postmortem brain and spinal cord tissues used in this study are deidentified by independent sources.

### 1.4.11 Immunofluorescent Staining and Immunohistochemistry

For immunofluorescent staining, mouse tissues were fixed in 4% paraformaldehyde and then sectioned at 20 μm on a cryostat. Slices were rinsed 3 times with PBS and treated with blocking solution (5% normal goat serum, 0.1% Tween 20 in 1× TBS) for 1 h at room temperature. Slices were incubated with a primary antibody (peIF2α, Cell Signaling; pPKCδ-⁵⁰⁵T, Cell Signaling; pMARK2-⁵⁹⁵T, Abcam) at 4° C. overnight. Then the slices were washed 3 times with PBS and incubated with a fluorochrome-conjugated secondary antibody (anti-rabbit, Alexa Fluor 594; Invitrogen, USA, 1:400) for 2 h at room temperature. After 3 to 5 times of washes with PBS, the slices were coverslipped in mounting medium containing DAPI. The ALS patient samples were fixed with 4% PFA prior to paraffin embedding. Paraffin-embedded tissue blocks were sectioned at 10 μm using a microtome. Tissue sections were mounted on Superfrost Plus slides, left to dry at room temperature for 24 h, and stored in −80° C. For use, the sections were heated at 65° C. for 30 min, cleared with xylene, deparaffinized, and hydrated through a series of graded anhydrous, histological grade ethanol solutions, then washed 3 times with xylenes and 100% EtOH, one time with the graded EtOH series of decreasing concentrations, and twice with deionized water. The sections were then rinsed with TBS and underwent antigen unmasking by incubating the slides in sub-boiling 10 mM citrate buffer (pH 6.0) for 10 min. Sections were cooled to room temperature and then underwent three 5-min washes with TBS. Endogenous peroxidase activity was quenched using a 10% methanol and 3% H₂O₂ solution in TBS for 10 min at room temperature. Afterwards, sections were washed twice with TBS and incubated with blocking buffer solution (0.3% Triton-X 100, 5% normal goat serum, 1% BSA in TBS) inside a humidified chamber for 30 min at room temperature. Blocking solution was aspirated, and sections were incubated with a primary antibody diluted in the blocking buffer solution (MARK2; 1:400) overnight at 4° C. The next day, after equilibrating to room temperature, sections were washed with TBST and treated with a micropolymerized peroxidase reporter, ImmPRESS Reagent Anti-Rabbit IgG (Vector Laboratories, USA), in a humidified chamber for 30 min at room temperature. The tissues were rinsed with TBST, and the peroxidase reporter was detected using the ImmPACT DAB (3,30-diaminobenzidine tetrahydrochloride) peroxidase substrate (Vector Laboratories). Tissue sections were incubated in the substrate working solution at room temperature until suitable staining developed, which was approximately 3 min. Slides were then rinsed again 3 times for 10 min in TBS and underwent dehydration with a series of graduated alcohols. Finally, the sections were cleared with three 5-min incubations in xylene and coverslipped with VectaMount Permanent Mounting Medium (Vector Laboratories).

### 1.4.12 Microscopy

Mouse tissue immunofluorescent staining was viewed with a Leica SP8 confocal fluorescence microscope. Z-stack images were taken and processed into a maximal projected image. Human samples stained by immunohistochemistry were viewed using brightfield microscopy on a Nikon Eclipse Ti—S microscope equipped with a high-definition color camera head, DS-Fi2, and DS-U3 control unit. Images were taken and assessed with NIS Elements Documentation Imaging Software (Nikon, USA) and analyzed using ImageJ software.

### 1.4.13 Statistical Analysis

The statistical analyses were performed with Student t tests for 2-group comparisons and one-way ANOVA with the Tukey post hoc test for multiple group comparisons using GraphPad Prism software. The sample size “n” represents independent experiments unless otherwise indicated. P values less than 0.05 were considered significant.

### Example 2

### Targeting an Eif2α Kinase to Regulate Translation Under Stress

2.1. MARK2 is a Direct Kinase for eIF2α

Phosphorylation of eIF2α is a key step in the translational attenuation that occurs in response to a variety of stresses in mammalian cells. Dever, 2002. To identify previously unrecognized eIF2α kinases, we searched a protein array dataset that suggested potential kinase and substrate relationships using microarrays composed of 4,191 unique human full-length proteins subjected to phosphorylation reactions with over 200 purified human kinases. Newman et al., 2013; Hu et al., 2009.

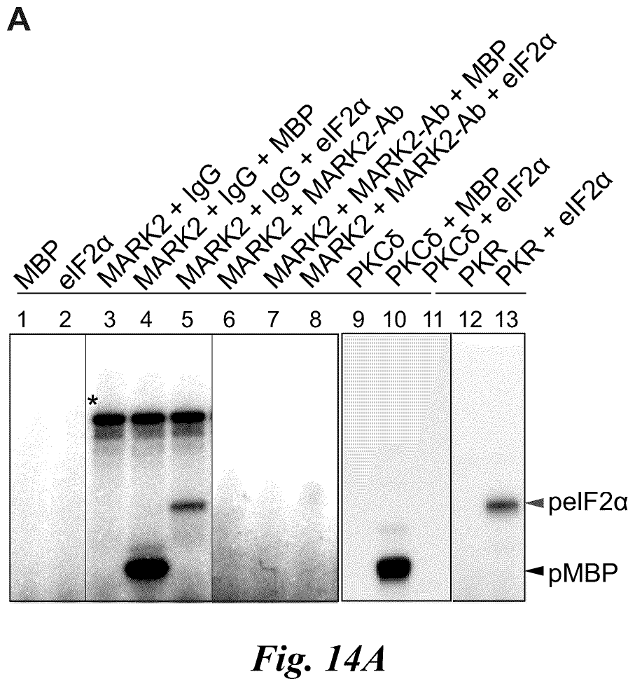

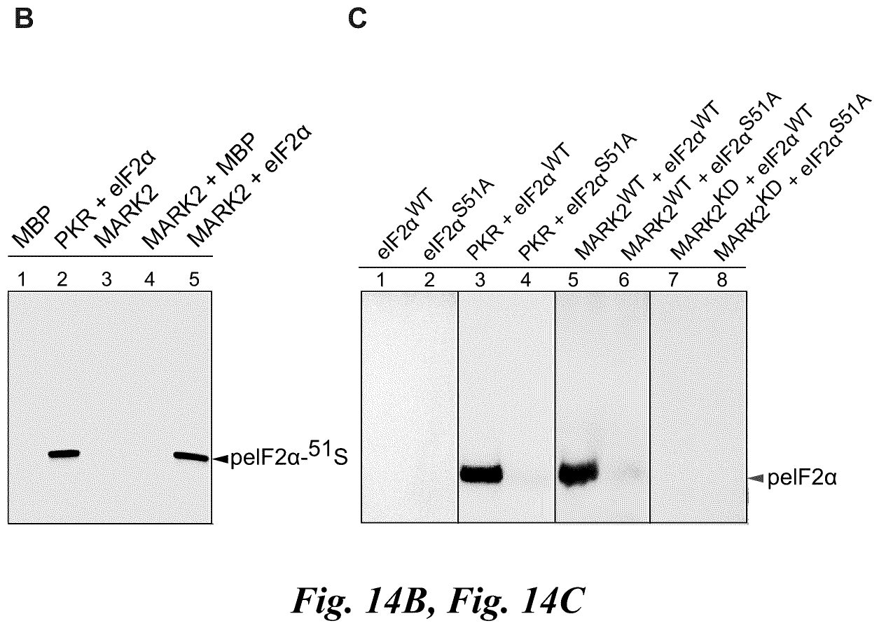

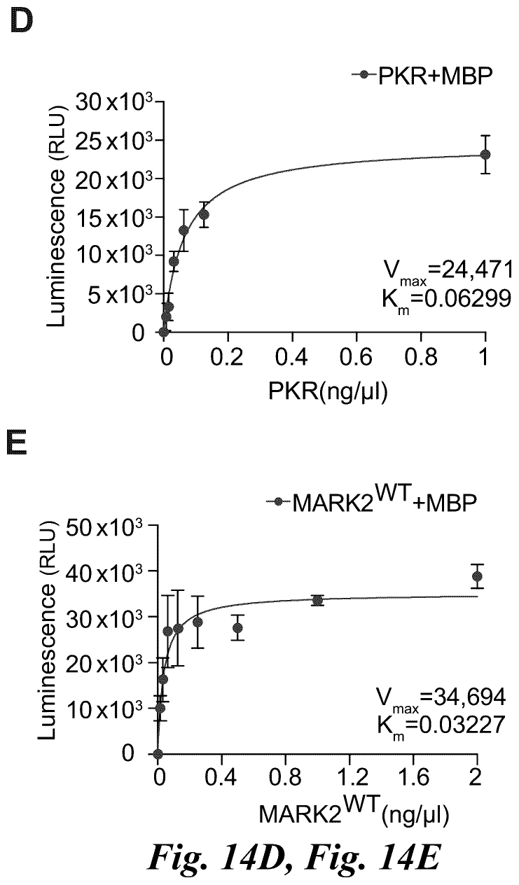

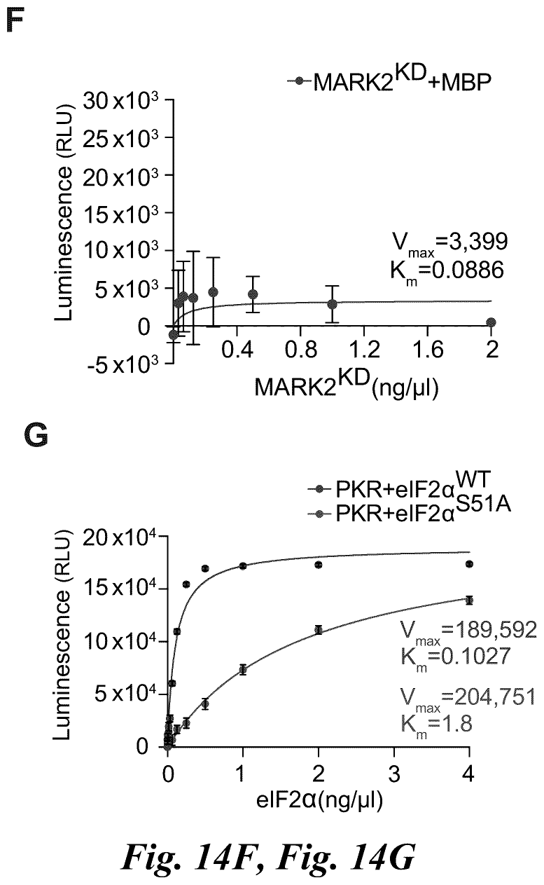

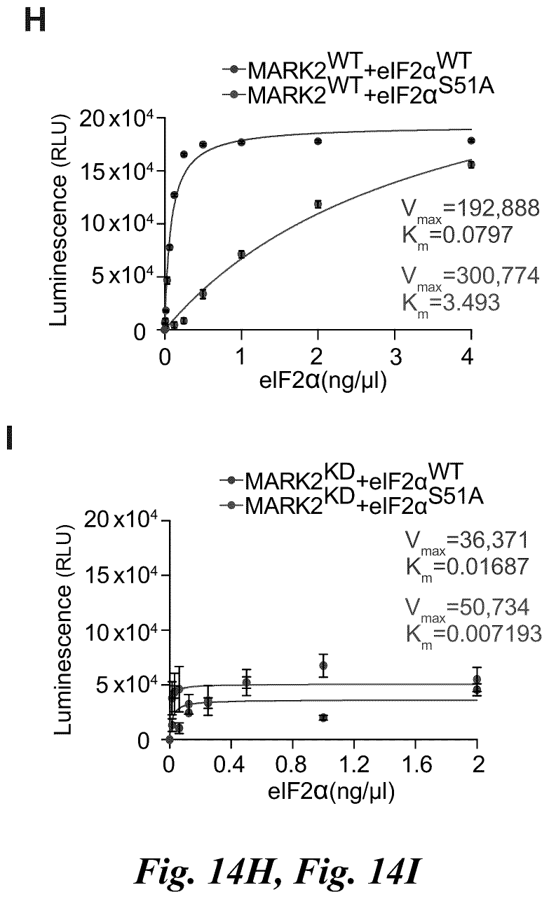

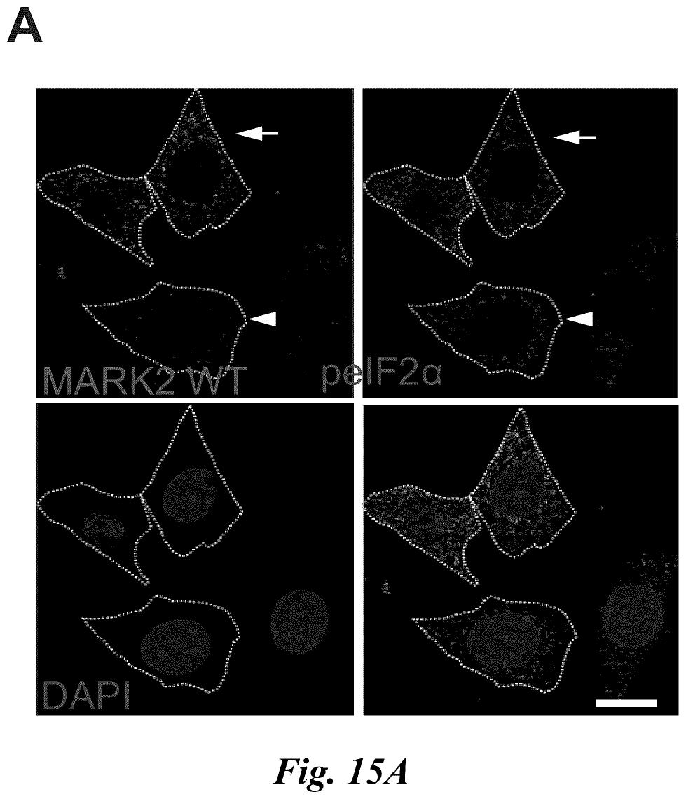

The protein array screen suggested at least four candidate kinases for eIF2α: protein tyrosine kinase 2 beta (PYK2), TTK protein kinase (TTK), bone morphogenetic protein receptor type 1A (BMPR1A), and MARK2. To determine which of these candidate kinases is capable of phosphorylating eIF2α, we performed in vitro kinase assays with radiolabeled ATP and proteins purified from Sf9 insect cells, including the eIF2α substrate and each of the four candidate kinases. Only MARK2 showed kinase activity, phosphorylating eIF2α in vitro (FIG. 14A). Myelin basic protein (MBP), a common substrate for diverse kinases, and PKR, a positive control kinase, were used to confirm that all the tested kinases were enzymatically active. Notably, the phosphorylation of eIF2α by MARK2 was completely blocked by a MARK2-specific antibody but not by an IgG control (FIG. 14A, lane 8), confirming that the observed activity for the eIF2α kinase was specifically associated with the MARK2 protein.

To characterize the kinase activity of MARK2 towards eIF2α, we purified a series of WT and mutant MARK2 and eIF2α proteins using an E. coli expression system and performed in vitro enzyme kinetics analyses. The Kinase-Glo assay was used to measure kinase activities by quantifying ATP consumption via luminescent signals. First, by using MBP as a shared substrate, we observed that MARK2 and the positive control kinase PKR showed similar reactivity as shown in Michaelis-Menten kinetics curves (FIG. 14D and FIG. 14E). Additionally, we generated a kinase-dead mutant MARK2′, that lacks the catalytic domain, Drewes et al., 1997, as a negative control and confirmed the absence of kinase activity for the mutant (FIG. 14F). Then, using the Km concentrations of MARK2 and PKR established above (FIG. 14D and FIG. 14E), we studied the kinetics of their kinase activity towards eIF2α. We found that MARK2 exhibited a robust kinase activity for eIF2α comparable to that of PKR, as evidenced by the similarity of Km and Vmax values in Michaelis-Menten kinetics curves between the two sets of reactions (FIG. 14G and FIG. 14H). As expected, the kinase-dead MARK2^{KD} mutant did not show activity towards eIF2α (FIG. 14I).

Translational control via the phosphorylation of eIF2α at serine 51 (⁵¹S) is a point of convergence for integrated stress response pathways. Dever, 2002. Using a phosphorylation-dependent antibody against phospho-eIF2α-⁵¹S, we showed that the radiolabeled phospho-eIF2α signal seen in the kinase assay with MARK2 was positively recognized by the antibody against the phosphorylated eIF2α-⁵¹S (FIG. 14B). To further validate the phosphorylation of eIF2α by MARK2 at serine 51, we purified the phosphor-null mutant eIF2α^{S51A}. Using the in vitro kinase assay based on ATP radiolabeling and gel electrophoresis, we confirmed that eIF2α^{S51A} was not phosphorylated, when compared to the WT form of the substrate, by either PKR or MARK2 (FIG. 14C). The kinase-dead MARK2′ mutant did not show activity towards eIF2α^{WT} or eIF2α^{S51A} (FIG. 14C). Consistently, in the kinetics analysis, the eIF2α^{S51A} mutant protein showed substantially lower reactivity for PKR or MARK2 than WT eIF2α, as evidenced by the curve slopes and Km values (FIG. 14G and FIG. 14H). Together, these results demonstrate that MARK2 directly phosphorylates eIF2α at serine 51.

2.2. MARK2 is a Kinase for eIF2α in Mammalian Cells

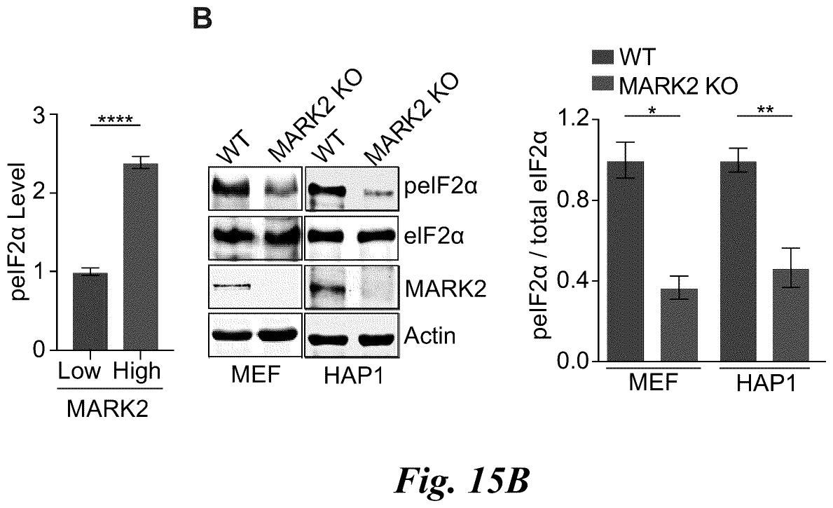

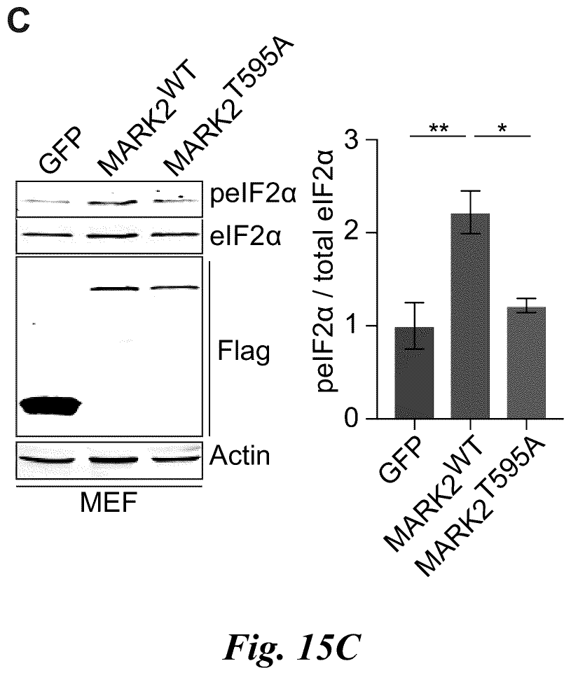

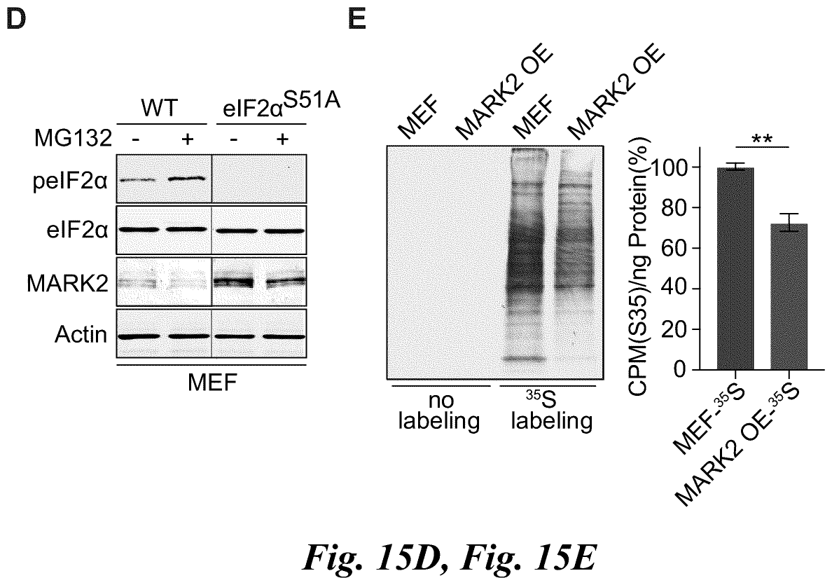

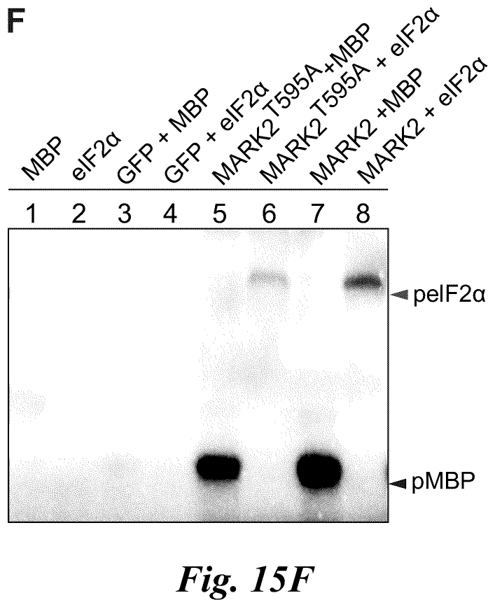

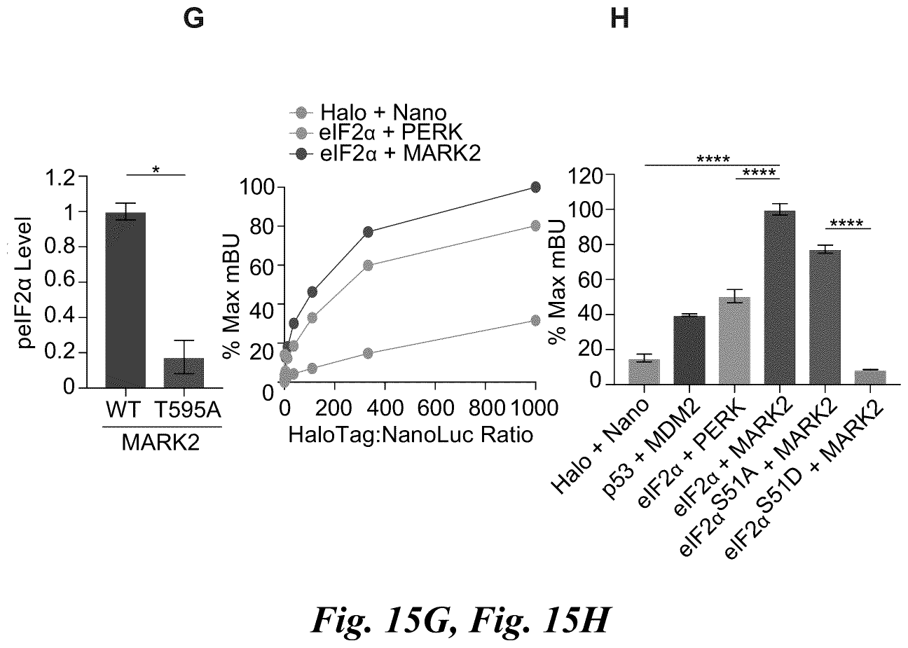

To study the physiologically relevant kinase activity of MARK2 in vivo, we analyzed the kinase activity of MARK2 on eIF2α in mammalian cells. Using mouse embryonic fibroblasts (MEFs), we compared cells expressing different levels of MARK2 for their correlation with the levels of phosphorylated eIF2α-⁵¹S: The cells with relatively higher levels of cytoplasmic MARK2 showed higher levels of phosphorylated eIF2α-⁵¹S, whereas the neighboring cells with less MARK2 showed lower levels of phosphorylated eIF2α-⁵¹S, as demonstrated by immunofluorescent staining for both MARK2 and eIF2α (FIG. 15A), suggesting that MARK2 positively regulates eIF2α phosphorylation in the cells. Next, we examined MARK2-mediated regulation of eIF2α phosphorylation by immunoblot analysis. Using MEFs in which the MARK2 locus was disrupted by the deletion of exons 2-4, Hurov et al., 2001, we found that the absence of MARK2 resulted in a significant reduction in the level of phosphorylated eIF2α-⁵¹S, without any detectable change in the level of total eIF2α protein (FIG. 15B). This finding was validated in human HAP1 cells in which the MARK2 gene was disrupted with a CRISPR-engineered premature stop codon in exon 2. Consistent with the result in MEFs, the human MARK2 knockout cells showed substantially lower levels of phosphorylated eIF2α-⁵¹S than did the WT control cells (FIG. 15B). Conversely, in MEFs that stably expressed elevated levels of MARK2, the phosphorylation of eIF2α-⁵¹S was significantly increased, without changing the level of total eIF2α protein (FIG. 15C). The specificity of the antibody against phosphorylated eIF2α-⁵¹S was verified in an eIF2α^{S51A} knock-in mutant MEF line, in which the S51A mutation abolished the immunoblot signal of phosphorylated eIF2α-⁵¹S (FIG. 15D). In line with the regulation of eIF2α phosphorylation by MARK2, we observed that elevated expression of MARK2 led to attenuation of global translation, as shown by reduction of the newly synthesized proteins, in MEFs metabolically labeled with ³⁵S-Methionine and ³⁵S-Cysteine and analyzed by autoradiography and liquid scintillation counting (FIG. 15E). Together, these results demonstrate that MARK2 promotes the phosphorylation of eIF2α-⁵¹S and influences translation in the cell.

MARK2 itself is phosphorylated, and the most-studied phosphorylation site is at its threonine 595 (T595) residue. Suzuki et al., 2004; Hurov et al., 2004. To test whether the kinase activity of MARK2 on eIF2α was dependent on its phosphorylation at T595, the T595 residue was mutated to alanine (A), and stable MEF lines were generated expressing the MARK2^{T595A} mutant. In the MEF cells, the MARK2^{T595A} mutant did not exhibit the ability to promote the phosphorylation of eIF2α-⁵¹S when compared to the MARK2^{WT} (FIG. 15C). To confirm this result, we performed in vitro kinase activity assays using WT and mutant MARK2 proteins purified from HEK293 cells. MARK2^{T595A} showed a much lower level of eIF2α kinase activity than did MARK2^{WT} in the radiolabeled eIF2 phosphorylation assay (FIG. 15F). These results indicate that the MARK2 kinase activity for eIF2α requires the phosphorylation of MARK2 at T595.

To confirm the direct interaction between MARK2 and eIF2α in live cells, we employed a proximity-based protein-protein interaction assay, NanoBRET, based on bioluminescence resonance energy transfer (BRET), to study the dynamic interaction between MARK2 and eIF2α in natural cellular environment. MARK2 was fused with the energy donor NanoLuc, and eIF2α was fused with the energy acceptor HaloTag; an interaction between MARK2 and eIF2α would bring the energy donor and acceptor into proximity and give rise to detectable BRET signals (FIG. 15G). When the MARK2 and eIF2α fusion proteins were co-expressed in HEK293 cells, a strong BRET signal was detected, while the unfused NanoLuc and HaloTag proteins served as the negative control and showed much lower background BRET signals. Donor saturation assays were performed to monitor the kinetics of the direct interaction between MARK2 and eIF2α in the live cells. The MARK2 and eIF2α pair showed a hyperbolic curve, indicating that the energy transfer value reached a maximum when all the donors were saturated with the acceptors (FIG. 15G). Interestingly, the interaction between MARK2 and eIF2α indicated by the BRET signal of the pair was stronger than that between the known kinase PERK and eIF2α, which itself was stronger than the non-specific interaction between unfused NanoLuc and HaloTag, in the donor saturation assay (FIG. 15G). Moreover, the interaction between MARK2 and eIF2α was stronger than that of another positive control interacting pair, p53 and MDM2, in the quantitative NanoBRET assay (FIG. 15H). To determine how phosphorylation of eIF2α at the ⁵¹S site affects its interaction with MARK2, we generated the phosphor-null and phosphor-mimicking mutants S51 A and S51D, respectively, for eIF2α and subjected them to the NanoBRET assay. Whereas the eIF2α^{S51A} mutant retained much of the interaction with MARK2, the eIF2α^{S51D} mutant showed no interaction with MARK2 (FIG. 15I), suggesting that the phosphorylation event diminishes the interaction between MARK2 and eIF2α. Collectively, these results support the notion that MARK2 is a direct and specific kinase of eIF2α at its serine 51 site both in vitro and in vivo.

2.3 MARK2 Mediates eIF2α Phosphorylation Independently of Previously Known Kinases

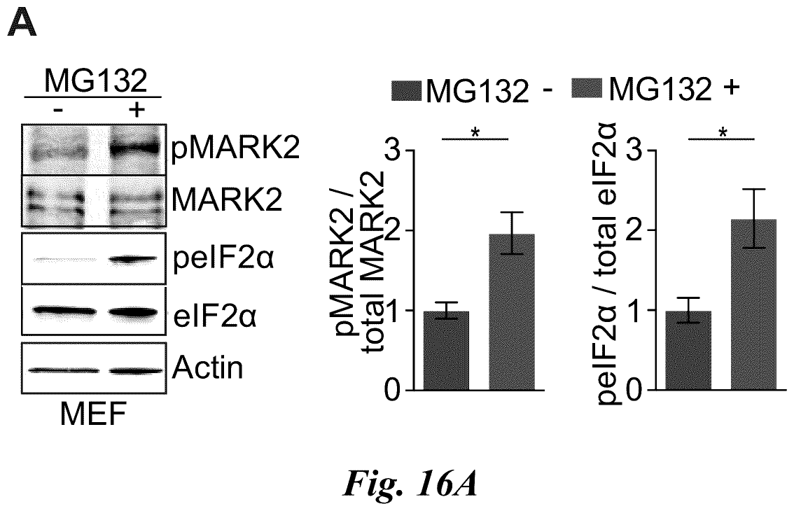

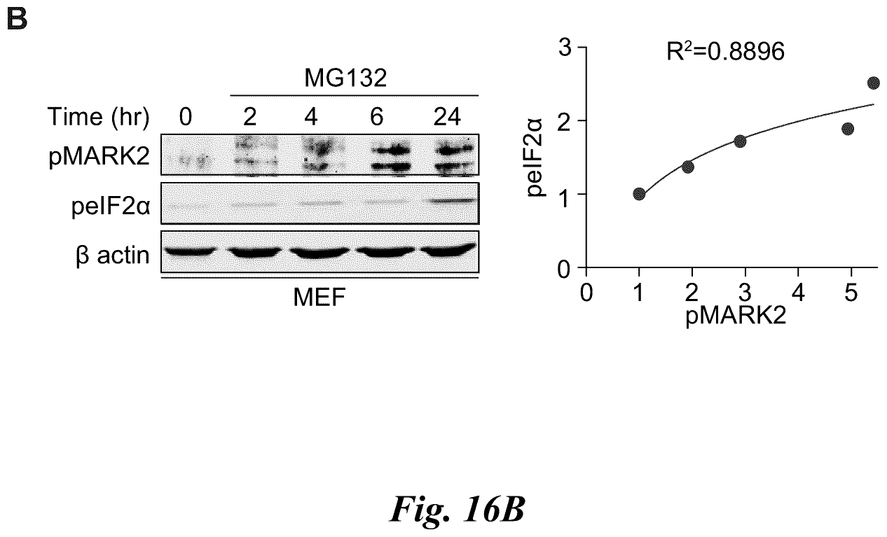

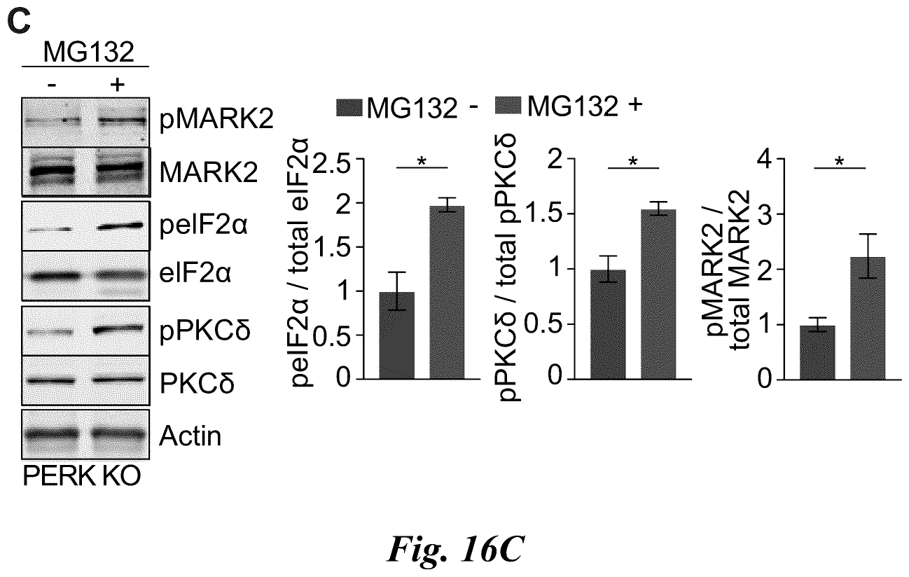

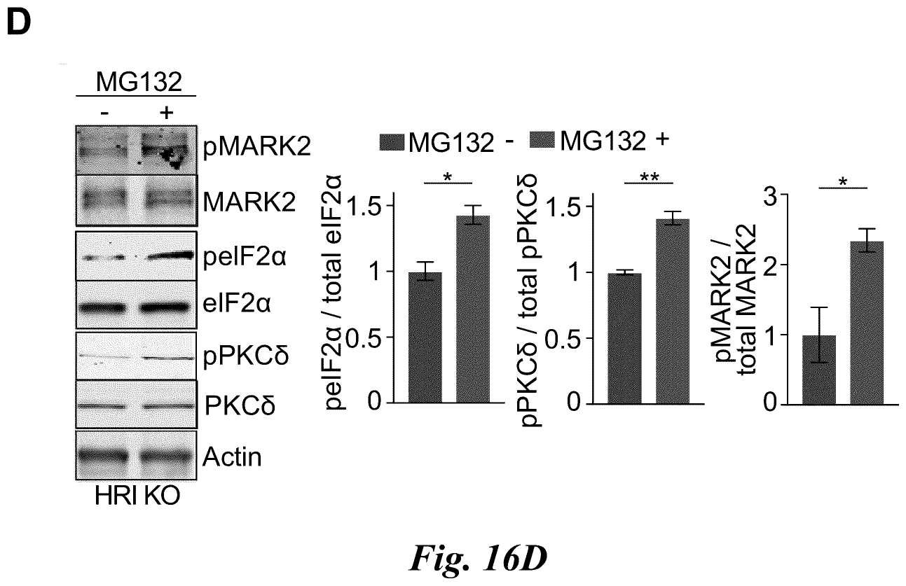

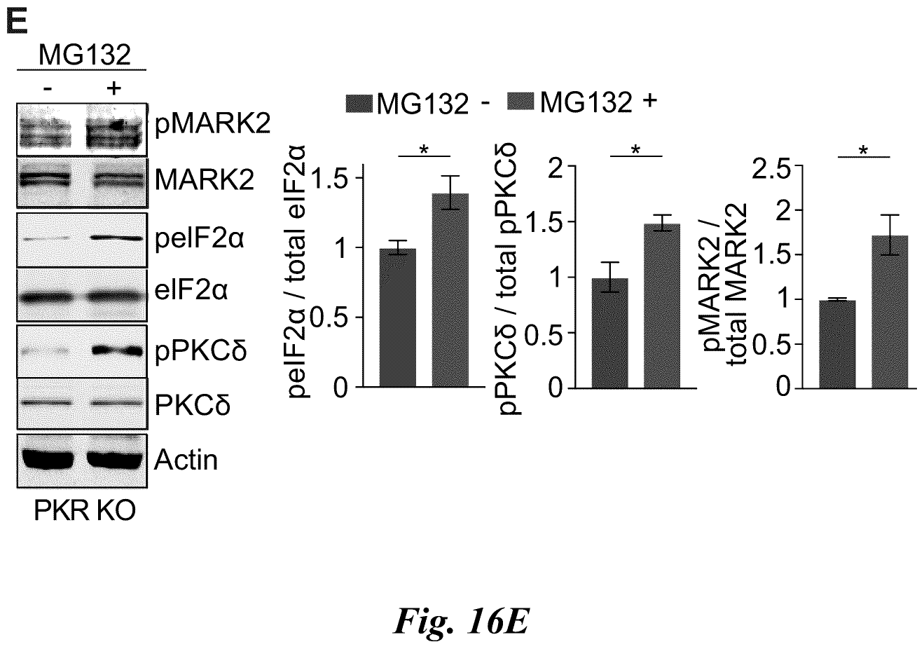

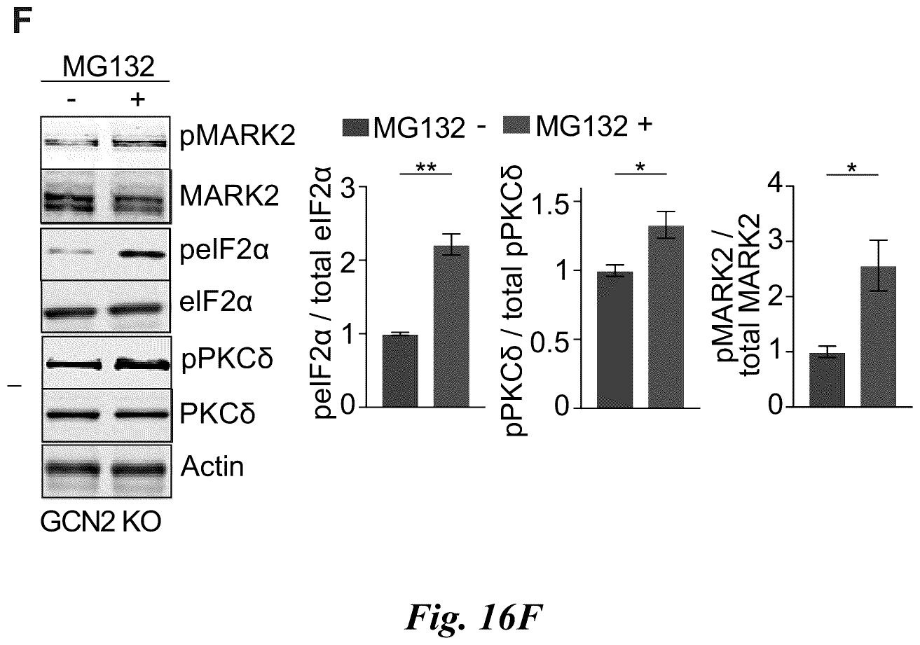

Since the phosphorylation of MARK2 at T595 is required for its positive regulation of eIF2α phosphorylation (FIG. 15C), we asked whether this form of phosphorylated MARK2 is regulated upon cytosolic protein misfolding stress. To induce the protein misfolding stress, we treated MEF cells with the proteasome inhibitor MG132, which elicited a substantial increase in the levels of phosphorylated MARK2-⁵⁹⁵T as well as a corresponding increase in the levels of phosphorylated eIF2α-⁵¹S (FIG. 16A). In a time course study using the treatment with 500 nM MG132 for 24 hr, the proteotoxic stressor led to a time-dependent increase in the levels of phosphorylated MARK2-⁵⁹⁵T, together with concomitantly increased levels of phosphorylated eIF2α-⁵¹S (FIG. 16B). These data suggest that the MARK2-eIF2α signaling pathway is activated in response to the proteotoxic stress.

Next we tested the activation of MARK2 by proteotoxic stress in the absence of PERK, HRI, PKR, or GCN2. In all four types of knockout MEFs lacking each of the four kinases, MARK2 was activated under the MG132-induced stress, as indicated by the increased phosphorylation at its 595T site (FIG. 16C-FIG. 16F). Accordingly, the phosphorylation of eIF2α-⁵¹S also was significantly increased (FIG. 16C-FIG. 16F). These data indicate that the activation of the MARK2-eIF2α pathway by the protein misfolding stress does not require any of the four previously known eIF2α kinases.

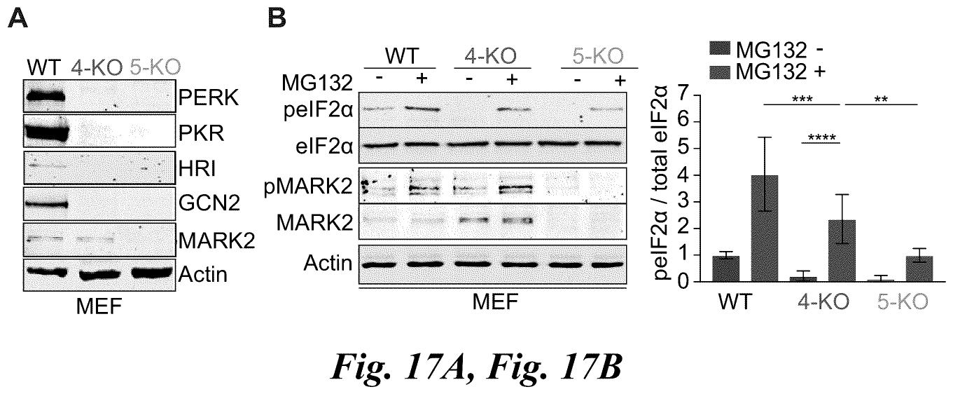

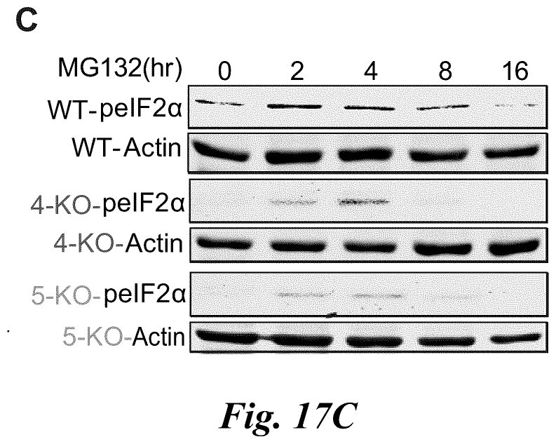

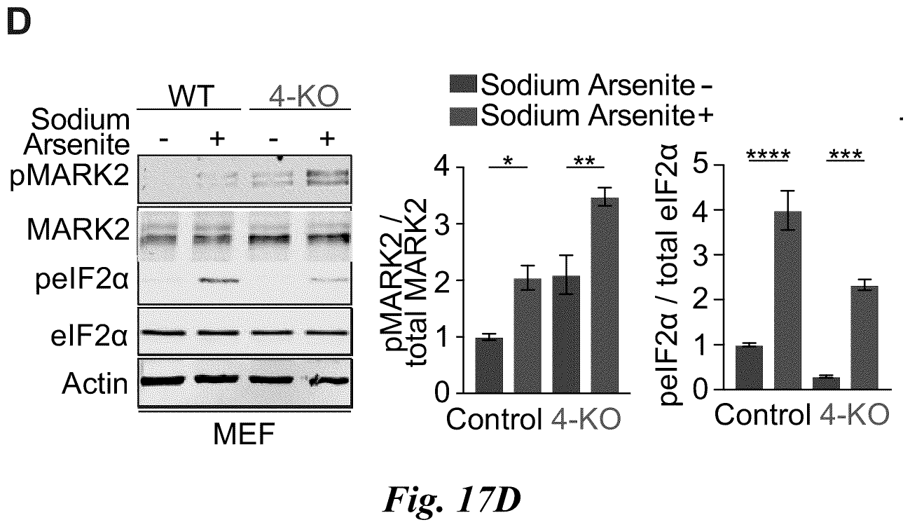

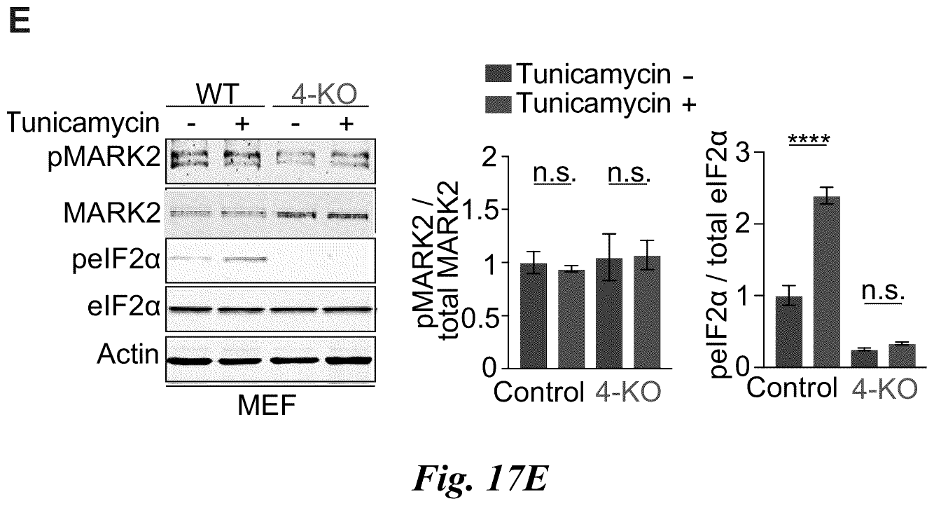

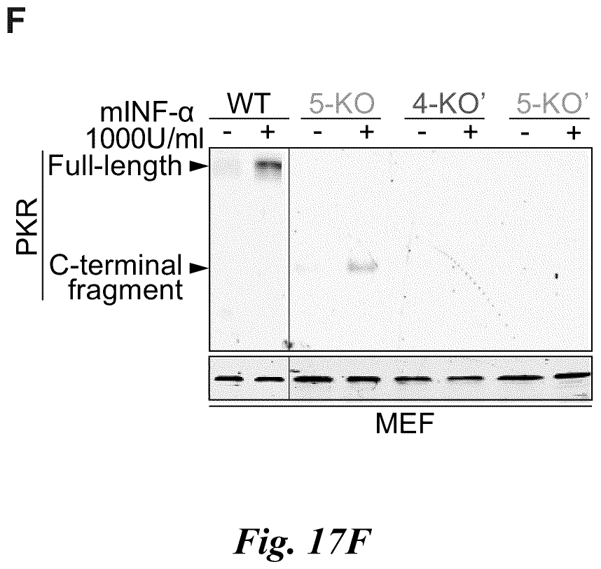

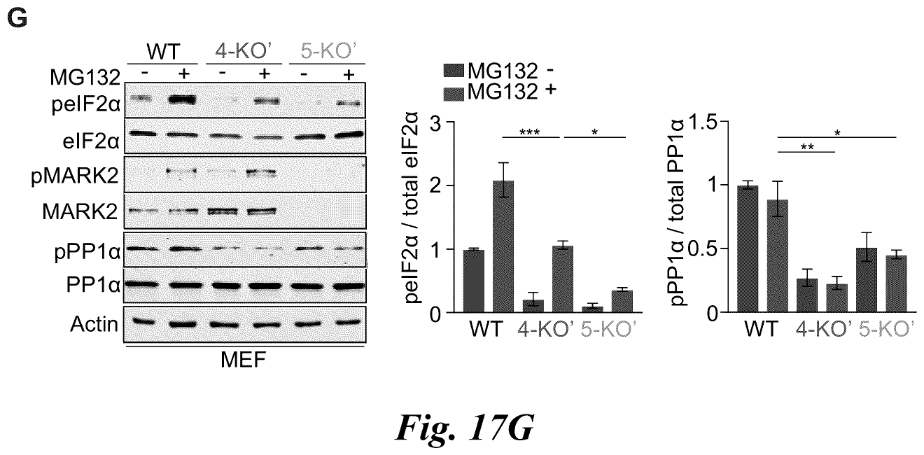

To further demonstrate that MARK2 alone is sufficient to promote the phosphorylation of eIF2α in the absence of all four previously known eIF2α kinases, we used multiplex CRISPR-Cas9 gene editing to knock out PERK, GCN2, and HRI in an existing PKR-knockout MEF line, Yang et al., 1995, creating 4-KO MEF lines (FIG. 17A). Next, we applied the proteasome inhibitor MG132 to the 4-KO MEF cells and found that the stress response, as indicated by eIF2α phosphorylation, was still intact. The phosphorylation of eIF2α-⁵¹S induced by the proteotoxic stress was lower in the 4-KO MEFs than in the WT MEFs (FIG. 17B); however, when compared to the unstressed cells, the phosphorylation of eIF2α in the absence of all four previously established kinases remained clearly detectable (FIG. 17B). In the 4-KO MEFs, the stress-induced change in the levels of phosphorylated eIF2α-⁵¹S was correlated with the increase in the level of MARK2-⁵⁹⁵T (FIG. 17B). To test whether MARK2 mediates the phosphorylation of eIF2α, we created independent 5-KO MEF lines by knocking out MARK2 in the 4-KO MEFs (FIG. 17B). When the 5-KO MEFs were treated with MG132, the phosphorylation of eIF2α was still enhanced, but the increase was significantly less than that in the 4-KO MEFs (FIG. 17B), indicating that MARK2 is capable of promoting eIF2α phosphorylation independently of the four previously known kinases. The condition of MG132 treatment at 20 μM for 4 hr was chosen for optimal induction of eIF2α phosphorylation in the analysis of these MEFs (FIG. 17C). To test whether activation of MARK2 also can be induced by other types of stress, we subjected the cells to oxidative stress, such as sodium arsenite treatment, or ER stress, such as tunicamycin treatment. The sodium arsenite treatment, known to cause protein damages throughout the cell, was able to induce the phosphorylation of MARK2-⁵⁹⁵T and eIF2α-⁵¹S in both WT MEFs and those lacking the four previously known kinases (FIG. 17D), consistent with the activation of MARK2 by proteotoxic stress. In comparison, the tunicamycin treatment, known to activate PERK, has no effect on the phosphorylation of MARK2-⁵⁹⁵T, induced the phosphorylation of eIF2-⁵¹S in WT MEFs but not in the cells lacking the four previously known kinases including PERK (FIG. 17E), consistent with the notion that MARK2 and ER stress act via independent pathways to regulate eIF2α phosphorylation.

It was reported that the PKR KO MEF line that we used to generate the 4-KO and 5-KO cells expresses a remnant C-terminal fragment of the PKR protein. Yang et al., 1995; Baltzis and Koromilas, 2002. We used CRISPR editing to create additional deletion mutations in the PKR gene in the exiting 4-KO and 5-KO cells (FIG. 17F) and confirmed the disruption of the remnant C-terminal fragment, as validated by the absence of its expression under interferon-α stimulation, in the newly generated 4-KO′ and 5-KO′ MEF lines (FIG. 17F). Examination of the phosphorylation of eIF2α-⁵¹S induced by MG132 in the 4-KO′ and 5-KO′ MEFs showed similar results as those in the 4-KO and 5-KO cells (FIG. 17G), confirming the MARK2-dependent phosphorylation of eIF2α in the absence of the four previously known kinases.

Furthermore, we examined the activity of protein phosphatase 1 (PP1), which catalyzes the dephosphorylation of eIF2α. Novoa et al., 2001; PP1a is phosphorylated at 320T, which inhibits its phosphatase activity. Kwon et al., 1997. We observed a decrease in the phosphorylation of PP1α-³²⁰T in 4-KO′ and 5-KO′ MEFs compared to WT MEFs, suggesting that the activation of PP1 contributes to the decrease in eIF2α phosphorylation in the 4-KO′ and 5-KO′ MEFs (FIG. 17G). Additionally, there was a trend for an increase in the phosphorylation of PP1α-³²⁰T in the 5-KO′ MEFs compared to the 4-KO′ MEFs (FIG. 17G). Since the reduced activity of PP1 in the 5-KO′ MEFs as compared to that in the 4-KO′ MEFs would enhance eIF2α phosphorylation in the 5-KO′ MEFs, the significant decrease in the phosphorylation of eIF2α-⁵¹S observed in the 5-KO′ MEFs as compared to the 4-KO′ MEFs, as a result of the loss of MARK2, supports the notion that MARK2 plays a major role in directly promoting eIF2α phosphorylation.

### 2.4 a PKCδ-MARK2-eIF2α Signaling Pathway in Response to Protein Misfolding Stress

To understand the regulation of MARK2 activation under conditions of protein misfolding, we sought to identify the upstream kinase that is responsible for activating MARK2 in response to the stress conditions. In the course of our studies, we tested PKCδ, a member of the atypical PKC family, as a potential kinase of MARK2, because PKCδ was shown to be activated during stress responses, and another member of the atypical PKC family was previously reported to phosphorylate MARK2 in the regulation of cell polarity. Suzuki et al., 2004; Hurov et al., 2004.

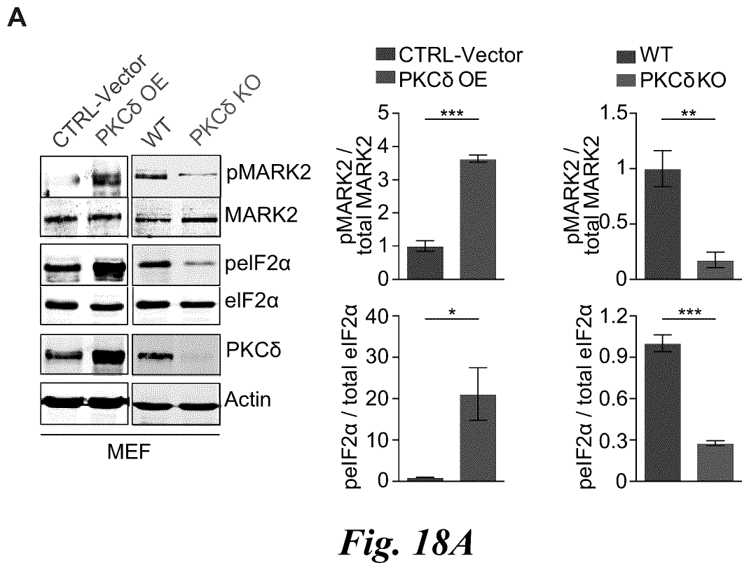

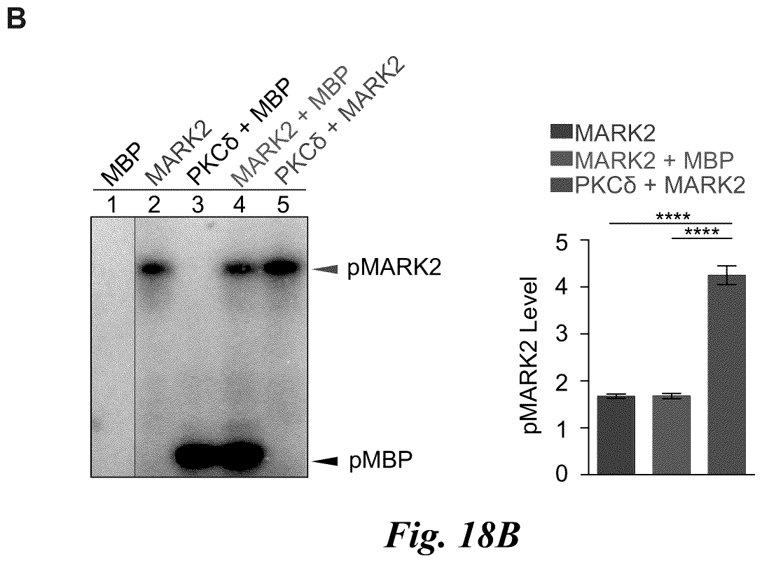

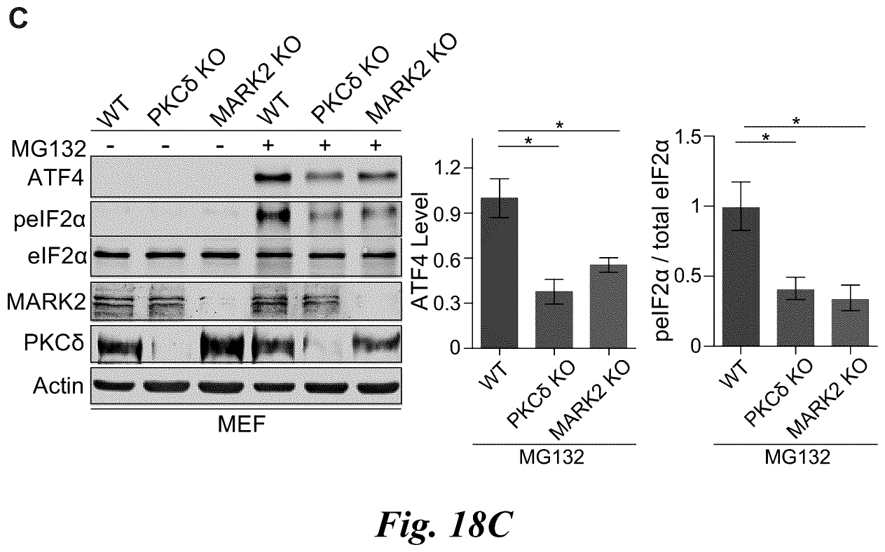

First, we generated MEF cell lines that stably expressed PKCδ and observed a substantial increase in the level of phosphorylated MARK2-⁵⁹⁵T, suggestive of the activation of MARK2 when the PKCδ level was elevated (FIG. 18A, left). The phosphorylation of eIF2α-⁵¹S was increased in correlation with that of MARK2-⁵⁹⁵T. Conversely, in PKCδ-knockout MEFs, the signals for phosphorylated MARK2, and accordingly that of eIF2α, were abolished (FIG. 18A, right). As controls, the total levels of MARK2 and eIF2α protein were not altered. Next, we asked whether PKCδ would exhibit any direct kinase activity toward MARK2. Using in vitro kinase assays with purified proteins, we found that PKCδ significantly increased the level of phosphorylated MARK2, with a background of autophosphorylation of MARK2, indicating that PKCδ has intrinsic kinase activity for MARK2 (FIG. 18B). In comparison, PKCδ did not show any kinase activity toward eIF2α in the in vitro kinase assay, indicating that PKCδ is not a direct kinase of eIF2α (FIG. 14A, lane 11). These results therefore point to a PKCδ-MARK2-eIF2α signaling pathway that operates in the cell.

To determine whether PKCδ is required for the activation of MARK2-eIF2α signaling downstream, we applied MG132-induced proteotoxic stress to MEF cells with or without PKCδ. In the WT MEFs, MG132 elicited a robust up-regulation of phosphorylated MARK2-⁵⁹⁵T, reflective of increased MARK2 kinase activity; concomitantly, we observed a parallel increase in the phosphorylation of eIF2α-⁵¹S (FIG. 18C). In contrast, in the PKCδ-knockout MEFs, we saw a substantially diminished increase in the levels of phosphorylated MARK2-⁵⁹⁵T or eIF2α-⁵¹S in response to MG132, indicating inhibition of MARK2 kinase activity towards eIF2α (FIG. 18C). Furthermore, consistent with the activation of the MARK2-eIF2α pathway by MG132 treatment in the knockout MEFs lacking each of the four known kinases of eIF2α (PERK, HRI, PKR, and GCN2), PKCδ also was activated, as demonstrated by the phosphorylation of its 505T site (FIG. 16C-FIG. 16F). Steinberg, 2004. This result indicated that the PKCδ-MARK2-eIF2α signaling is independent of the previously known eIF2α kinases.

Although the phosphorylation of eIF2α leads to attenuation of the translation of most transcripts, it also increases the translation of specific mRNAs such as activating transcription factor 4 (ATF4), Harding et al., 2000, as part of the stress response. Indeed, treatment of MEF cells with MG132, which induces proteotoxicity, increased the level of the ATF4 protein (FIG. 15G). The increase in the ATF4 level, however, was significantly reduced in knockout MEFs lacking PKCδ or MARK2 (FIG. 18C). Accordingly, the levels of eIF2α phosphorylation were significantly reduced in the absence of PKCδ or MARK2 (FIG. 18C). These observations demonstrate that PKCδ and MARK2 are major positive regulators of eIF2α phosphorylation during MG132-induced proteotoxic stress, providing evidence for a PKCδ-MARK2-eIF2α signaling cascade.

2.5 HSP90 Interacts with PKCδ and Mediates Stress-Dependent Activation of PKCδ

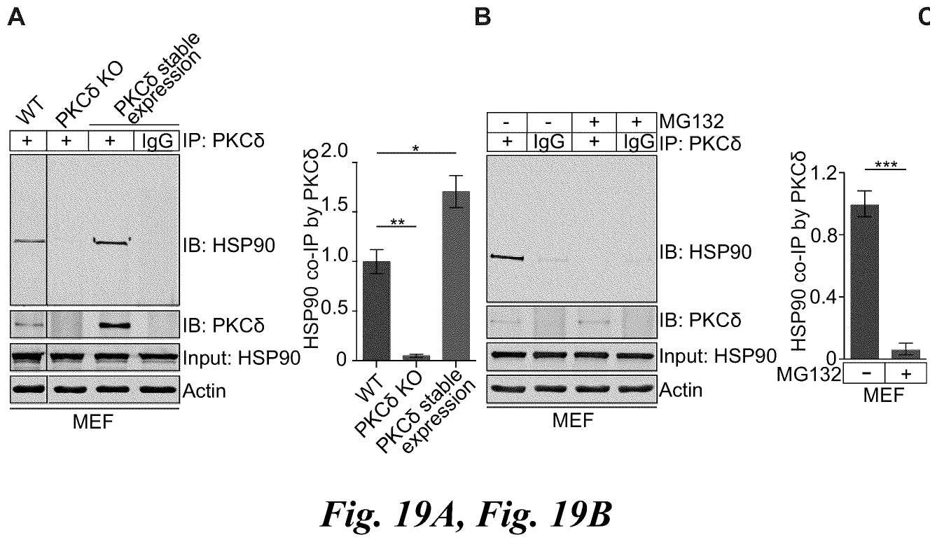

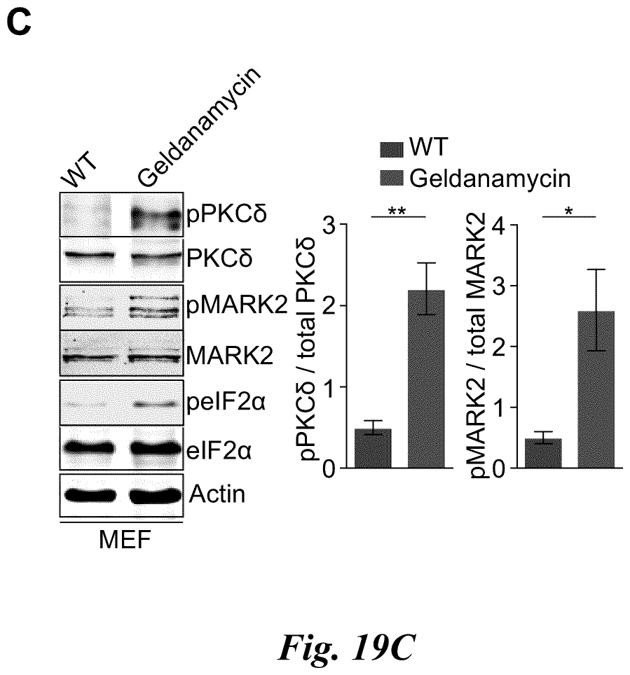

We next investigated how the PKCδ-MARK2-eIF2α signaling pathway senses protein misfolding stress. Since misfolded proteins, including SOD1, are prone to interact with molecular chaperone proteins, Tummala et al., 2005; Wang et al., 2009, we reasoned that the upstream regulator PKCδ could potentially interact with a molecular chaperone and that disruption of this interaction by misfolded proteins during proteotoxic stress might be a mechanism to account for the activation of the PKCδ kinase. We searched a protein-protein interaction database, Szklarczyk et al., 2015, and identified several candidate interactors with PKCδ, including HSP90, HSP90a, and HSP70. To evaluate potential interactions between PKCδ and these molecular chaperones, we performed co-immunoprecipitation experiments with endogenous proteins in MEF cells. After immunoprecipitation of PKCδ, only HSP90 was pulled down by PKCδ (FIG. 19A). When the level of PKCδ protein was elevated via stable expression in MEF cells, more HSP90 protein could be coimmunoprecipitated by PKCδ. Conversely, depletion of PKCδ from MEFs or use of an IgG control instead of the anti-PKCδ antibody abolished the co-immunoprecipitation of HSP90, confirming the specific interaction between PKCδ and HSP90 (FIG. 19A). To test whether increased levels of misfolded proteins could disrupt the interaction between PKCδ and HSP90, we performed co-immunoprecipitation assays in MEF cells treated with MG132. We found that the MG132-induced proteotoxic stress completely abolished the interaction between PKCδ and HSP90, suggesting that the accumulated misfolded proteins compete for the binding of HSP90 and release PKCδ from the complex (FIG. 19B).

To determine whether HSP90 regulates the activation of PKCδ, we tested the effect in MEF cells of a specific inhibitor of HSP90, geldanamycin, Whitesell et al., 1994, on the phosphorylation of PKCδ at residue threonine 505 (505T), which is required for the kinase activity of PKCδ. Steinberg, 2004. We found that this HSP90 inhibitor substantially enhanced the phosphorylation of PKCδ-⁵⁰⁵T (FIG. 19C). Concomitant with the activation of PKCδ, there was an increase in the phosphorylation of MARK2-⁵⁹⁵T and eIF2α-⁵¹S in the cells treated with the HSP90 inhibitor (FIG. 19C). These data demonstrate that inhibition of HSP90 results in the activation of the PKCδ-MARK2-eIF2α signaling pathway. Collectively, these results suggest that PKCδ and HSP90 form a complex that can sense protein-misfolding stress through the intrinsic affinity of HSP90 for misfolded proteins and thereby regulate the activation of PKCδ-MARK2-eIF2α signaling.

2.6 Dysregulated phosphorylation of PKCδ-⁵⁰⁵T and MARK2-⁵⁹⁵T in ALS mice and patients

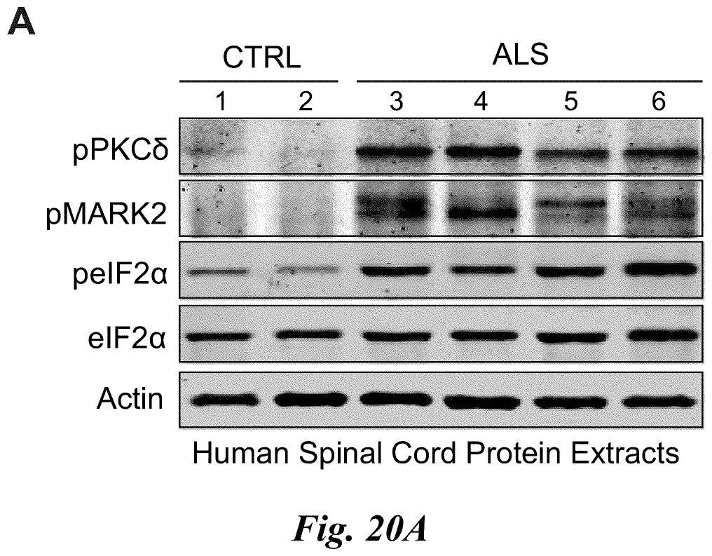

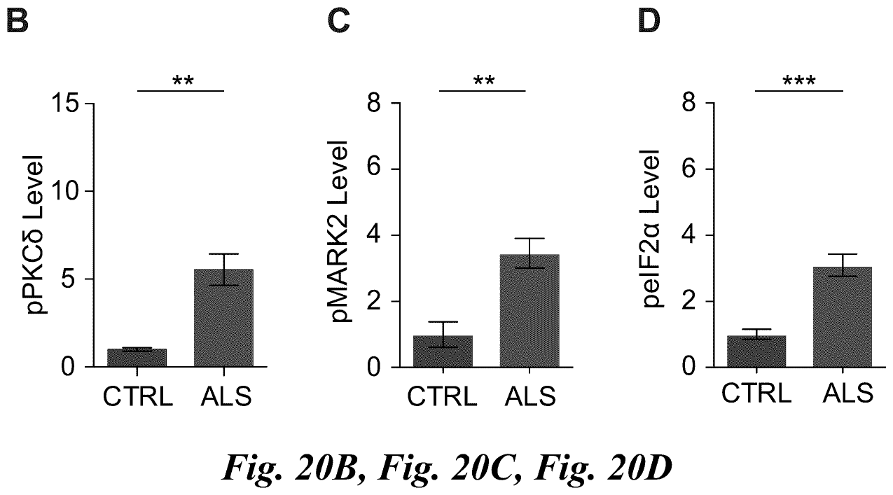

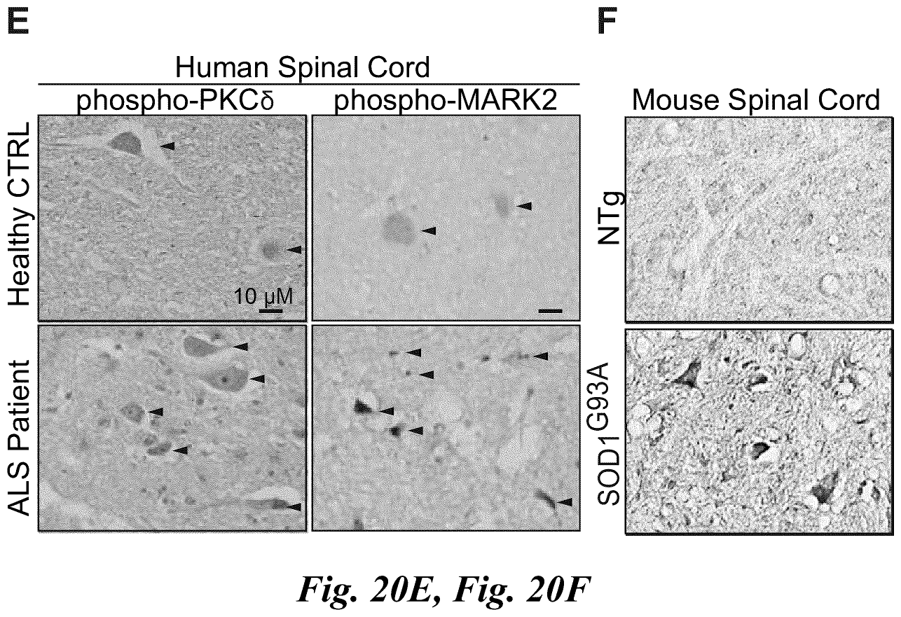

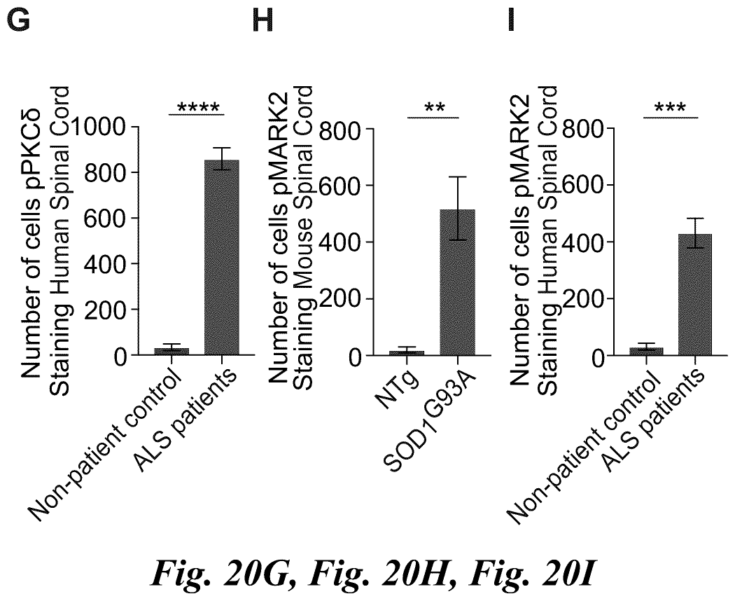

Next, we examined the status of the PKCδ-MARK2-eIF2α signaling pathway in ALS mouse models and patients. To extend our findings to human patients, we performed immunoblot analysis of PKCδ and MARK2 in spinal cord tissues from ALS patients and healthy controls. Of the thirteen ALS patients' spinal cords examined, most showed a substantial increase in the phosphorylation of PKCδ-⁵⁰⁵T when compared to healthy controls (FIG. 20A and FIG. 20B), suggesting that activation of PKCδ in the spinal cord is a general feature in ALS patients. In the patients' tissues that showed up-regulated phosphorylation of PKCδ-⁵⁰⁵T, the phosphorylation of MARK2-⁵⁹⁵T also was significantly increased (FIG. 20A, FIG. 20C). The increases in the phosphorylation of PKCδ and MARK2 were correlated with the increase in the phosphorylation of eIF2α-⁵¹S in the patients' spinal cords (FIG. 20A, FIG. 20D). Furthermore, we performed immunohistochemical staining of the spinal cords from ALS patients including one carrying the mutation SOD1^{A4V} using antibodies against phosphorylated PKCδ-⁵⁰⁵T and confirmed that the PKCδ phosphorylation was markedly increased in the affected spinal cords (FIG. 20E and FIG. 20G). These results demonstrate that the increase in the catalytically active form of PKCδ, as marked by phosphorylated PKCδ-⁵⁰⁵T, is a pathological consequence of mutant SOD1 in mammalian systems.

We also investigated the neuropathology of MARK2 in ALS patients or animal models. In non-transgenic control animals, immunostaining with an antibody specific for MARK2-⁵⁹⁵T showed a diffuse pattern in the gray matter of the anterior horn of the spinal cord. In age-matched symptomatic SOD1^{G93A} mice, however, the staining was markedly increased in both the gray and white matter (FIG. 20F and FIG. 20H). Furthermore, we studied the pattern of phosphorylated MARK2 in human spinal cord tissues. In heathy control subjects, the immunohistochemical staining of phosphorylated MARK2 was relatively light, except for a set of well-demarcated large neurons. In contrast, in the familial ALS patient with the SOD1^{A4V} mutation, the signals for phosphorylated MARK2 in the cells were increased in both number and intensity (FIG. 20E and FIG. 20I). These results suggest that the activation of MARK2 is a notable pathologic feature in the spinal cords of ALS patients who suffer from the motor neuron degeneration.

2.7 a Specific Inhibitor for Inhibiting MARK2 Kinase Activity and Reducing the Phosphorylation of eIF2α

In the present study, we identified the MARK2 as a direct kinase for eIF2α in a stress-response signaling. Next, we aimed to develop specific inhibitors that decrease MARK2 kinase functions and inhibit the MARK2-eIF2α signaling. To our knowledge, there is only one compound that is claimed to inhibit MARK family members, and there is no specific inhibitors known for the MARK2 kinase. To develop novel specific inhibitors for the MARK2 kinase, we first examined the structure and function of the existing MARK family inhibitor and found that this compound not only inhibits the MARK family but also shows inhibits the GSK3β function. A previous report showed that the GSK3β inhibitors SB-216763 and SB-415286 had cross-reactivity with MARK2 and also inhibited MARK kinase function. Bain et al., 2007. Another report showed that an inhibitor of GSK3β inhibited the activities of MARK family members. Timm et al., 2011.

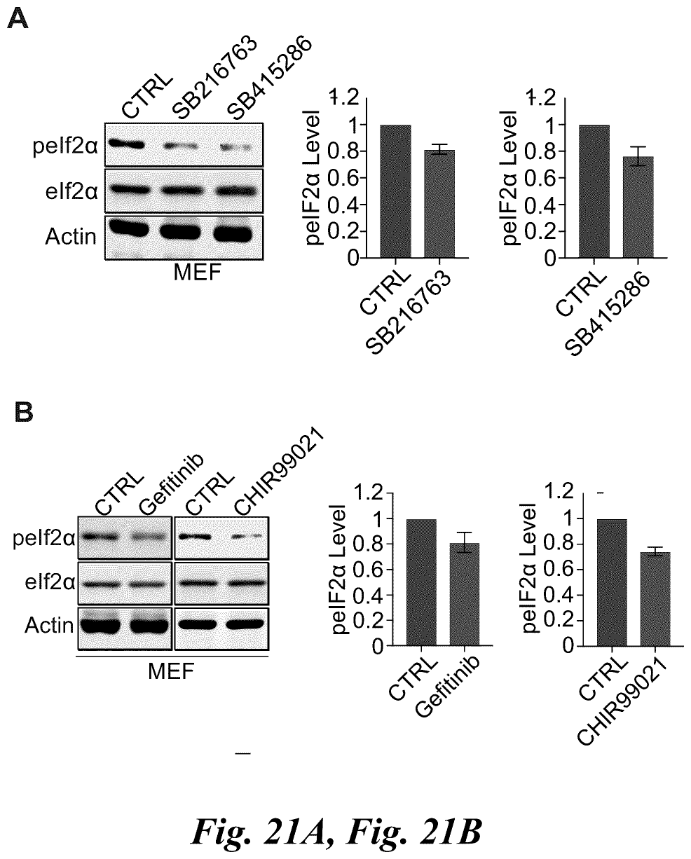

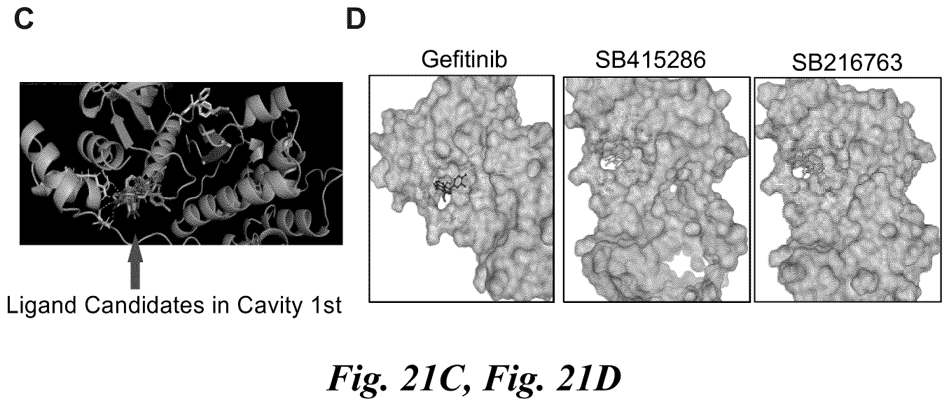

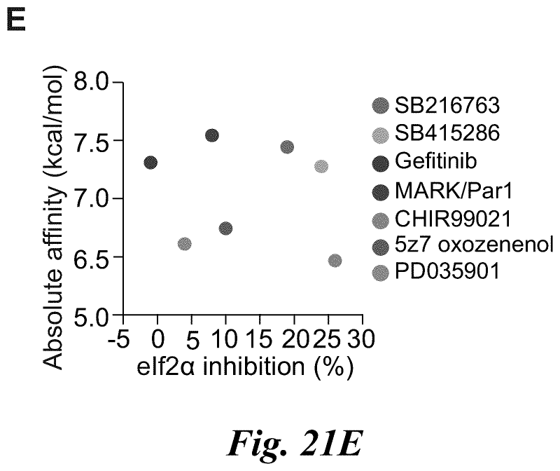

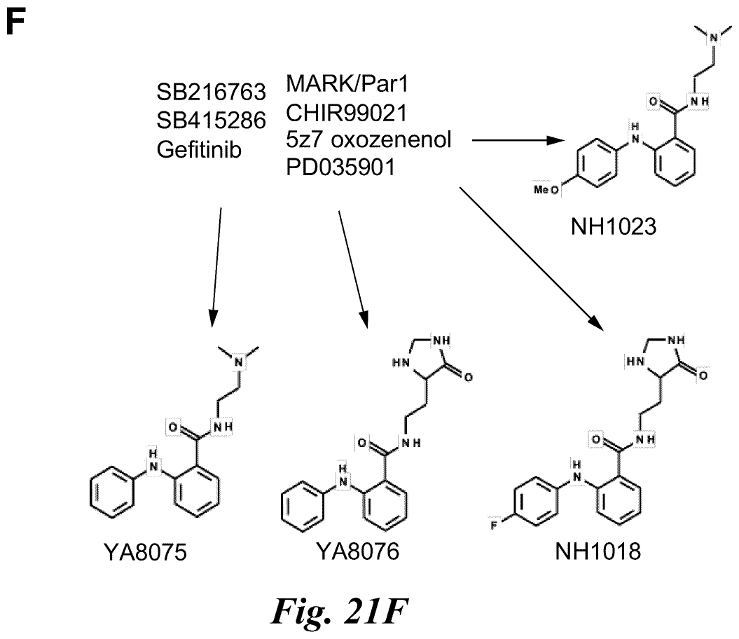

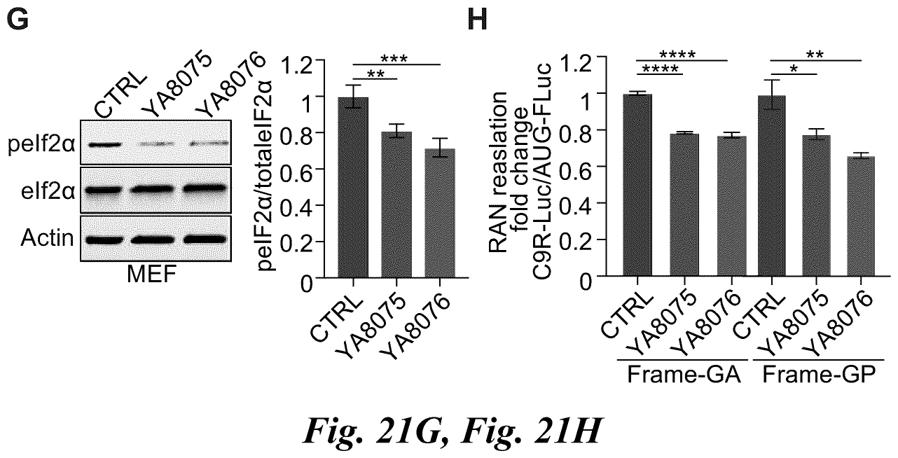

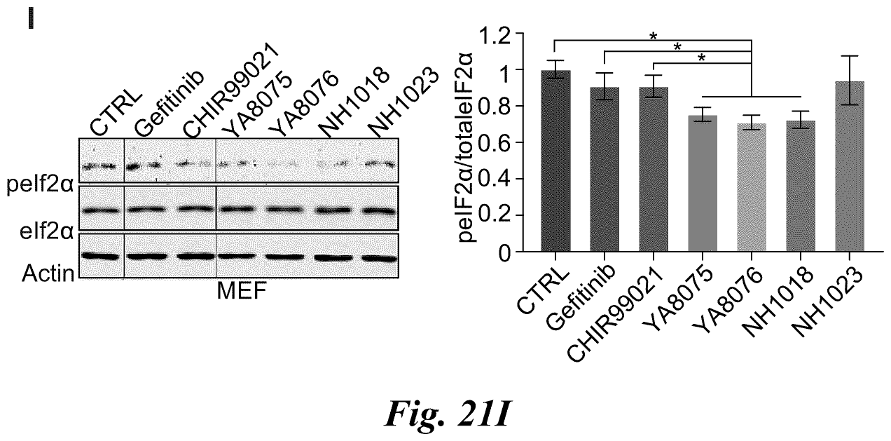

Therefore, we used six different GSK3β inhibitors that also were shown to have some levels of activities of inhibiting MARK2 and performed a comparative analysis of their efficacies in the cell co-treatment with MG132. Among them, SB-216763 and SB-415286 substantially diminished the increase in the levels of phosphorylated eIF2α-⁵¹S in response to MG132, suggesting that these drugs inhibited the MARK2 kinase activity towards eIF2α (FIG. 21A). We tested 5 additional GSK3β drugs and found that at low concentrations CHIR99021 had an effect of substantially lowering the levels of phosphorylated eIF2α-⁵¹S (FIG. 21B). We also analyzed these drugs' structures and interactions with MARK2 protein through computational methods and found that they have a similar interaction site that is close to the end of the kinase domain and the N-terminus of the UBA domain (FIG. 21C-FIG. 21D). We also compared all the seven different drugs and the decreased phosphorylation of eIF2α and made the following observations: CHIR99021 had a high activity in inhibiting the phosphorylation of eIF2α but its affinity with MARK2 protein was lower than those of other drugs; SB-216763 and SB-415286 had high activity in inhibiting the phosphorylation of eIF2α and high affinity for MARK2 protein; Gefitinib showed low activity in inhibiting the phosphorylation of eIF2α but had a high affinity for MARK2 protein (FIG. 21E).

Based on these results, we designed five novel compounds with different structures, including YA8075, NH1010, NH1023, YA8076, and NH1018, in the hope of achieving high activity in inhibiting the phosphorylation of eIF2α and high affinity for MARK2 (FIG. 21F). In a cell-based MARK2 kinase activity assay based on the eIF2α phosphorylation under MG132-induced stress, we found YA8075, YA8076, and NH1018 had similar effects on decreasing the phosphorylation level of eIF2α, with 20% reduction of phosphorylated eIF2α for YA8075, 30% for YA8076, and 27% for NH1018 (FIG. 21G, FIG. 21I). Indeed, the new drugs' activities in reducing phosphorylation level of eIF2α are higher than those of the existing MARK family inhibitors (FIG. 21I). In addition to the three new drugs that showed activities in reducing eIF2α phosphorylation, we also found a new compound, NH1023, which showed no effects on eIF2α phosphorylation compared with the control group, suggesting the specificity of the effective inhibitors. Previously, increased phosphorylation of eIF2α was associated with the RAN translation at the C9 or f72 hexanucleotide repeat expansion, the most common cause of neurodegenerative diseases ALS/FTD, which produces toxic peptides. Cheng et al., 2018; Tusi et al., 2021. To test the potential therapeutic effects of our MARK2 inhibitors against RAN translation, we used a RAN translation reporter cell line and treated it with YA8075 or YA8076 in the presence of the MG132-induced stress. The results showed that the treatment with these two inhibitors robustly inhibited MG132-induced phosphorylation of eIF2α (FIG. 21G, 21I) and reversed the RAN translation induced by MG132 (FIG. 21H). These results demonstrate that our newly designed MARK2 inhibitor compounds YA8075, YA8076, and NH1018 have high efficacy in decreasing the phosphorylation eIF2α and could be used as better tools for investigating the functions of MARK2 and its potential as a therapeutic target.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.
Abdel-Nour M, Carneiro LAM, Downey J, Tsalikis J, Outlioua A, Prescott D, et al. The heme-regulated inhibitor is a cytosolic sensor of protein misfolding that controls innate immune signaling. Science. 2019; 365(6448). Epub Jul. 6, 2019.
Bain J, Plater L, Elliott M, Shpiro N, Hastie C J, McLauchlan H, et al. The selectivity of protein kinase inhibitors: a further update. The Biochemical journal. 2007; 408(3):297-315. Epub Sep. 14, 2007.
Balch W E, Morimoto R I, Dillin A, Kelly J W. Adapting Proteostasis for Disease Intervention. Science. 2008; 319(5865):916-9.
Baltzis D, Li S, Koromilas A E. Functional characterization of pkr gene products expressed in cells from mice with a targeted deletion of the N terminus or C terminus domain of PKR. J Biol Chem. 2002; 277 (41):38364-72. Epub Aug. 6, 2002.
Basu A, Pal D. Two faces of protein kinase Cdelta: the contrasting roles of PKCdelta in cell survival and cell death. ScientificWorldJournal. 2010; 10:2272-84.
Bertolotti A, Zhang Y, Hendershot L M, Harding H P, Ron D. Dynamic interaction of BiP and ER stress transducers in the unfolded-protein response. Nat Cell Biol. 2000; 2(6):326-32. Epub Jun. 15, 2000.
Bosco D A, Morfini G, Karabacak N M, Song Y, Gros-Louis F, Pasinelli P, et al. Wild-type and mutant SOD1 share an aberrant conformation and a common pathogenic pathway in ALS. Nat Neurosci. 2010; 13(11):1396-403.
Breunig C T, Durovic T, Neuner A M, Baumann V, Wiesbeck M F, Koferle A, et al. One step generation of customizable gRNA vectors for multiplex CRISPR approaches through string assembly gRNA cloning (STAgR). PLoS ONE. 2018; 13(4):e0196015. Epub Apr. 28, 2018.
Chang R C, Wong A K, Ng H K, Hugon J. Phosphorylation of eukaryotic initiation factor-2alpha (eIF2alpha) is associated with neuronal degeneration in Alzheimer's disease. Neuroreport. 2002; 13 (18):2429-32.
Chen J J, London I M. Regulation of protein synthesis by heme-regulated eIF-2 alpha kinase. Trends Biochem Sci. 1995; 20(3):105-8.
Chen J J, Throop M S, Gehrke L, Kuo I, Pal J K, Brodsky M, et al. Cloning of the cDNA of the heme-regulated eukaryotic initiation factor 2 alpha (eIF-2 alpha) kinase of rabbit reticulocytes: homology to yeast GCN2 protein kinase and human double-stranded-RNA-dependent eIF-2 alpha kinase. Proc Natl Acad Sci USA. 1991; 88(17):7729-33.
Cheng W, Wang S, Mestre A A, Fu C, Makarem A, Xian F, Hayes L R, Lopez-Gonzalez R, Drenner K, Jiang J, Cleveland D W, Sun S. C9ORF72 GGGGCC repeat-associated non-AUG translation is upregulated by stress through eIF2α phosphorylation. Nat Commun. 2018; 9(1):51.
Christianson J C, Ye Y. Cleaning up in the endoplasmic reticulum: ubiquitin in charge. Nat Struct Mol Biol. 2014; 21(4):325-35. https://doi.org/10.1038/nsmb.2793 PMID: 24699081.
Crosby J S, Lee K, London I M, Chen J J. Erythroid expression of the heme-regulated eIF-2 alpha kinase. Mol Cell Biol. 1994; 14(6):3906-14. Epub Jun. 1, 1994.
Deng H-X, Hentati A, Tainer J A, Iqbal Z, Cayabyab A, Hung W-Y, et al. Amyotrophic lateral sclerosis and structural defects in Cu,Zn superoxide dismutase. Science. 1993; 261:1047-51.
Dever T E, Feng L, Wek R C, Cigan A M, Donahue T F, Hinnebusch A G. Phosphorylation of initiation factor 2 alpha by protein kinase GCN2 mediates gene-specific translational control of GCN4 in yeast. Cell. 1992; 68(3):585-96.
Dever T E. Gene-specific regulation by general translation factors. Cell. 2002; 108(4):545-56.
Drewes G, Ebneth A, Preuss U, Mandelkow E M, Mandelkow E. MARK, a novel family of protein kinases that phosphorylate microtubule-associated proteins and trigger microtubule disruption. Cell. 1997; 89(2):297-308. Epub Apr. 18, 1997.
Feng G S, Chong K, Kumar A, Williams B R. Identification of double-stranded RNA-binding domains in the interferon-induced double-stranded RNA-activated p68 kinase. Proc Natl Acad Sci USA. 1992; 89 (12):5447-51.
Gurney M E, Pu H, Chiu A Y, Dal Canto M C, Polchow C Y, Alexander D D, et al. Motor neuron degeneration in mice that express a human Cu,Zn superoxide dismutase mutation. Science. 1994; 264:1772-5.
Harding H P, Novoa I, Zhang Y, Zeng H, Wek R, Schapira M, et al. Regulated translation initiation controls stress-induced gene expression in mammalian cells. Molecular Cell. 2000; 6(5):1099-108. Epub Dec. 7, 2000.
Harding H P, Zhang Y, Ron D. Protein translation and folding are coupled by an endoplasmic-reticulum-resident kinase. Nature. 1999; 397(6716):271-4.
Holcik M, Sonenberg N. Translational control in stress and apoptosis. Nat Rev Mol Cell Biol. 2005; 6 (4):318-27. Epub Apr. 2, 2005.
Hu S, Xie Z, Onishi A, Yu X, Jiang L, Lin J, et al. Profiling the human protein-DNA interactome reveals ERK2 as a transcriptional repressor of interferon signaling. Cell. 2009; 139(3):610-22. Epub Nov. 3, 2009.
Humphries M J, Limes and KH, Schneider J C, Nakayama K I, Anderson S M, Reyland M E. Suppression of apoptosis in the protein kinase Cdelta null mouse in vivo. J Biol Chem. 2006; 281(14):9728-37.
Hurov J B, Stappenbeck T S, Zmasek C M, White L S, Ranganath S H, Russell J H, et al. Immune system dysfunction and autoimmune disease in mice lacking Emk (Par-1) protein kinase. Mol Cell Biol. 2001; 21(9):3206-19.
Hurov J B, Watkins J L, Piwnica-Worms H. Atypical PKC phosphorylates PAR-1 kinases to regulate localization and activity. Curr Biol. 2004; 14(8):736-41.
Jackson D N, Foster D A. The enigmatic protein kinase Cdelta: complex roles in cell proliferation and survival. FASEB J. 2004; 18(6):627-36.
Jiang K, Liu Y, Fan J, Epperly G, Gao T, Jiang J, et al. Hedgehog-regulated atypical PKC promotes phosphorylation and activation of Smoothened and Cubitus interruptus in Drosophila. Proc Natl Acad Sci USA. 2014; 111(45):E4842-50. Epub Oct. 29, 2014.
Kikkawa U, Matsuzaki H, Yamamoto T. Protein kinase C delta (PKC delta): activation mechanisms and functions. J Biochem. 2002; 132(6):831-9.
Kikuchi H, Almer G, Yamashita S, Guegan C, Nagai M, Xu Z, et al. Spinal cord endoplasmic reticulum stress associated with a microsomal accumulation of mutant superoxide dismutase-1 in an ALS model. Proc Nati Acad Sci USA. 2006; 103(15):6025-30.
Kim H J, Raphael A R, LaDow E S, McGurk L, Weber R A, Trojanowski J Q, et al. Therapeutic modulation of eIF2alpha phosphorylation rescues TDP-43 toxicity in amyotrophic lateral sclerosis disease models. Nat Genet. 2014; 46(2):152-60. Epub Dec. 18, 2013.
Kutner R H, Zhang X Y, Reiser J. Production, concentration and titration of pseudotyped HIV-1-based lentiviral vectors. Nat Protoc. 2009; 4(4):495-505. Epub Mar. 21, 2009.
Kwon Y G, Lee S Y, Choi Y, Greengard P, Nairn A C. Cell cycle-dependent phosphorylation of mammalian protein phosphatase 1 by cdc2 kinase. Proc Natl Acad Sci U.S.A. 1997; 94(6):2168-73. Epub Mar. 18, 1997.
Lindberg M J, Bystrom R, Boknas N, Andersen P M, Oliveberg M. Systematically perturbed folding patterns of amyotrophic lateral sclerosis (ALS)-associated SOD1 mutants. Proc Natl Acad Sci USA. 2005; 102(28):9754-9.
Ma T, Trinh M A, Wexler A J, Bourbon C, Gatti E, Pierre P, et al. Suppression of eIF2alpha kinases alleviates Alzheimer's disease-related plasticity and memory deficits. Nat Neurosci. 2013; 16(9):1299-305.
Manning G, Whyte D B, Martinez R, Hunter T, Sudarsanam S. The protein kinase complement of the human genome. Science. 2002; 298(5600):1912-34. Epub Dec. 10, 2002.
McEwen E, Kedersha N, Song B, Scheuner D, Gilks N, Han A, et al. Heme-regulated inhibitor kinase-mediated phosphorylation of eukaryotic translation initiation factor 2 inhibits translation, induces stress granule formation, and mediates survival upon arsenite exposure. J Biol Chem. 2005; 280(17):16925-33. Epub Feb. 3, 2005.
Miller C J, Turk B E. Homing in: Mechanisms of Substrate Targeting by Protein Kinases. Trends Biochem Sci. 2018; 43(5):380-94. Epub Mar. 17, 2018.
Moreno J A, Radford H, Peretti D, Steinert J R, Verity N, Martin M G, et al. Sustained translational repression by eIF2alpha-P mediates prion neurodegeneration. Nature. 2012; 485(7399):507-11.
Neumann M, Sampathu D M, Kwong L K, Truax A C, Micsenyi M C, Chou T T, et al. Ubiquitinated TDP-43 in Frontotemporal Lobar Degeneration and Amyotrophic Lateral Sclerosis. Science. 2006; 314 (5796):130-3.
Newman R H, Hu J, Rho H S, Xie Z, Woodard C, Neiswinger J, et al. Construction of human activity-based phosphorylation networks. Mol Syst Biol. 2013; 9:655.
Nishitoh H, Kadowaki H, Nagai A, Maruyama T, Yokota T, Fukutomi H, et al. ALS-linked mutant SOD1 induces ER stress- and ASK1-dependent motor neuron death by targeting Derlin-1. Genes Dev. 2008; 22(11):1451-64.
Novoa I, Zeng H, Harding H P, Ron D. Feedback inhibition of the unfolded protein response by GADD34-mediated dephosphorylation of eIF2alpha. J Cell Biol. 2001; 153(5):1011-22. Epub May 31, 2001.
Periz G, Lu J, Zhang T, Kankel M W, Jablonski A M, Kalb R, et al. Regulation of Protein Quality Control by UBE4B and LSD1 through p53-Mediated Transcription. PLoS Biol. 2015; 13(4):e1002114. Epub Apr. 4, 2015.
Prostko C R, Dholakia J N, Brostrom M A, Brostrom C O. Activation of the double-stranded RNA-regulated protein kinase by depletion of endoplasmic reticular calcium stores. J Biol Chem. 1995; 270 (11):6211-5.
Prusiner S B. Cell biology. A unifying role for prions in neurodegenerative diseases. Science. 2012; 336 (6088):1511-3. Epub Jun. 23, 2012.
Renton A E, Chio A, Traynor B J. State of play in amyotrophic lateral sclerosis genetics. Nat Neurosci. 2014; 17(1):17-23.
Ron D, Walter P. Signal integration in the endoplasmic reticulum unfolded protein response. Nat Rev Mol Cell Biol. 2007; 8(7):519-29. Epub Jun. 15, 2007.
Rosen D R, Siddique T, Patterson D, FIG.lewicz DA, Sapp P, Hentati A, et al. Mutations in Cu/Zn superoxide dismutase gene are associated with familial amyotrophic lateral sclerosis. Nature. 1993; 362:59-62.
Rutkowski D T, Kaufman R J. That which does not kill me makes me stronger: adapting to chronic ER stress. Trends Biochem Sci. 2007; 32(10):469-76.
Ryu E J, Harding H P, Angelastro J M, Vitolo O V, Ron D, Greene L A. Endoplasmic reticulum stress and the unfolded protein response in cellular models of Parkinson's disease. J Neurosci. 2002; 22 (24):10690-8.
Scheuner D, Song B, McEwen E, Liu C, Laybutt R, Gillespie P, et al. Translational control is required for the unfolded protein response and in vivo glucose homeostasis. Mol Cell. 2001; 7(6):1165-76. Epub Jun. 30, 2001.
Sreedharan J, Blair I P, Tripathi V B, Hu X, Vance C, Rogelj B, et al. TDP-43 Mutations in Familial and Sporadic Amyotrophic Lateral Sclerosis. Science. 2008; 319(5870):1668-72.
Steinberg S F. Distinctive activation mechanisms and functions for protein kinase Cdelta. Biochem J. 2004; 384(Pt 3):449-59.
Suzuki A, Hirata M, Kamimura K, Maniwa R, Yamanaka T, Mizuno K, et al. aPKC acts upstream of PAR-1b in both the establishment and maintenance of mammalian epithelial polarity. Curr Biol. 2004; 14(16):1425-35. Epub Aug. 25, 2004.
Szklarczyk D, Franceschini A, Wyder S, Forslund K, Heller D, Huerta-Cepas J, et al. STRING v10: protein-protein interaction networks, integrated over the tree of life. Nucleic Acids Res. 2015; 43(Database issue):D447-52.
Taniuchi S, Miyake M, Tsugawa K, Oyadomari M, Oyadomari S. Integrated stress response of vertebrates is regulated by four eIF2alpha kinases. Sci Rep. 2016; 6:32886. Epub Sep. 17, 2016.
Timm T, von Kries J P, Li X, Zempel H, Mandelkow E, Mandelkow E M. Microtubule affinity regulating kinase activity in living neurons was examined by a genetically encoded fluorescence resonance energy transfer/fluorescence lifetime imaging-based biosensor: inhibitors with therapeutic potential. J Biol Chem. 2011; 286(48):41711-22. Epub Oct. 11, 2011.
Tummala H, Jung C, Tiwari A, Higgins C M, Hayward L J, Xu Z. Inhibition of chaperone activity is a shared property of several Cu,Zn-superoxide dismutase mutants that cause amyotrophic lateral sclerosis. 2005; 280(18):17725-31.
Tusi S K, Nguyen L, Thangaraju K, Li J, Cleary J D, Zu T, Ranum LPW. The alternative initiation factor eIF2A plays key role in RAN translation of myotonic dystrophy type 2 CCUG•CAGG repeats. Hum Mol Genet. 2021; 30(11):1020-1029.
Wang J, Farr G, Zeiss C, Rodriguez-Gil D, Wilson J, Furtak K, et al. Progressive aggregation despite chaperone associations of a mutant SOD1-YFP in transgenic mice that develop ALS. Proc Natl Acad Sci USA. 2009; 106(5):1392-7.
Wang J, Slunt H, Gonzales V, Fromholt D, Coonfield M, Copeland N G, et al. Copper-binding-site-null SOD1 causes ALS in transgenic mice: aggregates of non-native SOD1 delineate a common feature. Hum Mol Genet. 2003; 12(21):2753-64.
Wang J, Xu G, Borchelt D. High molecular weight complexes of mutant superoxide dismutase 1: age-dependent and tissue-specific accumulation. Neurobiol Dis. 2002; 9(2):139-48.
Wang L, Popko B, Roos R P. An enhanced integrated stress response ameliorates mutant SOD1-induced ALS. Hum Mol Genet. 2014; 23(10):2629-38.
Whitesell L, Mimnaugh E G, De Costa B, Myers C E, Neckers L M. Inhibition of heat shock protein HSP90-pp60v-src heteroprotein complex formation by benzoquinone ansamycins: essential role for stress proteins in oncogenic transformation. Proc Natl Acad Sci USA. 1994; 91(18):8324-8.
Yang Y L, Reis L F, Pavlovic J, Aguzzi A, Schafer R, Kumar A, et al. Deficient signaling in mice devoid of double-stranded RNA-dependent protein kinase. EMBO J. 1995; 14(24):6095-106. Epub Dec. 15, 1995.
Yerlikaya A, Kimball S R, Stanley B A. Phosphorylation of eIF2alpha in response to 26S proteasome inhibition is mediated by the haem-regulated inhibitor (HRI) kinase. Biochem J. 2008; 412(3):579-88. Epub Feb. 23, 2008.
Zhang F, Zhu H. Intracellular conformational alterations of mutant SOD1 and the implications for fALS-associated SOD1 mutant induced motor neuron cell death. Biochim Biophys Acta. 2006; 1760(3):404-14. Epub Jan. 25, 2006.
Zhang P, McGrath B C, Reinert J, Olsen D S, Lei L, Gill S, et al. The GCN2 eIF2alpha kinase is required for adaptation to amino acid deprivation in mice. Mol Cell Biol. 2002; 22(19):6681-8. Epub Sep. 7, 2002.
Zhong Y, Wang J, Henderson M J, Yang P, Hagen B M, Siddique T, et al. Nuclear export of misfolded SOD1 mediated by a normally buried NES-like sequence reduces proteotoxicity in the nucleus. Elife. 2017; 6. Epub May 4, 2017.
Zou J, Guo Y, Guettouche T, Smith D F, Voellmy R. Repression of Heat Shock Transcription Factor HSF1 Activation by HSP90 (HSP90 Complex) that Forms a Stress-Sensitive Complex with HSF1. Cell. 1998; 94(4):471-80.

Although the foregoing subject matter has been described in some detail by way of illustration and example for purposes of clarity of understanding, it will be understood by those skilled in the art that certain changes and modifications can be practiced within the scope of the appended claims.

1. A compound of formula (I): 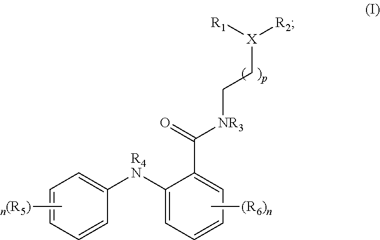
wherein:
m is an integer selected from 0, 1, 2, 3, 4, and 5;
n is an integer selected from 0, 1, 2, 3, and 4;
p is an integer selected from 0, 1, 2, 3, 4, 5, 6, and 7;
X is N or CH;
R₁ and R₂ are each independently selected from substituted or unsubstituted C₁-C₄ straight-chain or branched alkyl; or R₁ and R₂ combine to form a 5-membered heterocyclic ring;
R₃ and R₄ are each independently H or C₁-C₄ alkyl;
each R₅ can be the same or different and is independently selected from H, halogen, C₁-C₄ alkyl, —CF₃, C₁-C₄ alkoxyl, hydroxyl, nitro, amino, alkylamino, dialkylamino, sulfate, cyano, carboxyl, and mercapto;
each R₆ can be the same or different and is each independently selected from H, halogen, C₁-C₄ alkyl, —CF₃, C₁-C₄ alkoxyl, hydroxyl, nitro, amino, alkylamino, dialkylamino, sulfate, cyano, carboxyl, and mercapto; and
pharmaceutically acceptable salts thereof.

2. The compound of claim 1, wherein the compound of formula (I) is a compound of formula (I-a): 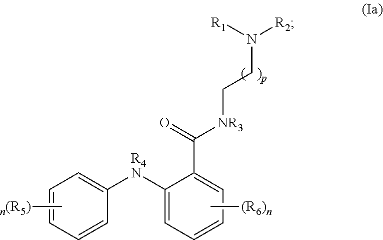
wherein R₁ and R₂ are each independently substituted or unsubstituted C₁-C₄ straight-chain or branched alkyl.

3. The compound of claim 2, wherein the compound of formula (I-a) is:

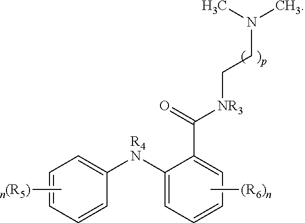

4. The compound of claim 3, wherein the compound of formula (I-a) is:

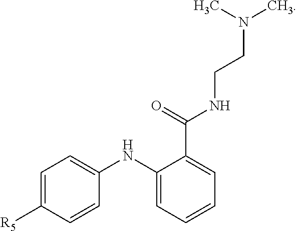

5. The compound of claim 4, wherein the compound of formula (I-a) is selected from:

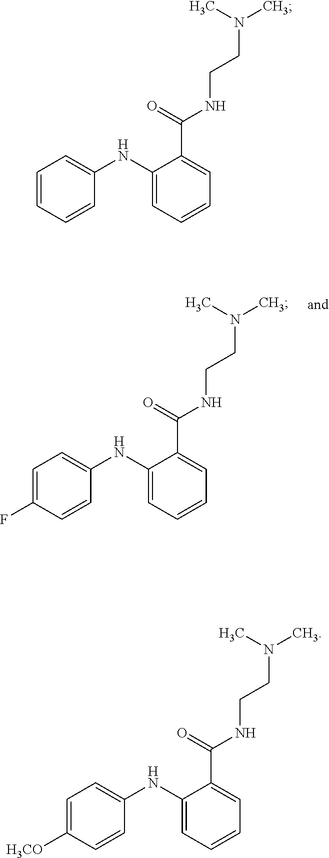 [SMILES: CN(C)CCNC(=O)c1ccccc1Nc1ccccc1 ; CN(C)CCNC(=O)c1ccccc1Nc1ccc(F)cc1 ; COc1ccc(Nc2ccccc2C(=O)NCCN(C)C)cc1]

6. The compound of claim 1, wherein the compound of formula (I) is a compound of formula (I-b):

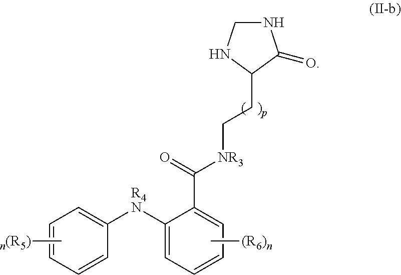

7. The compound of claim 6, wherein the compound of formula (I-b) is:

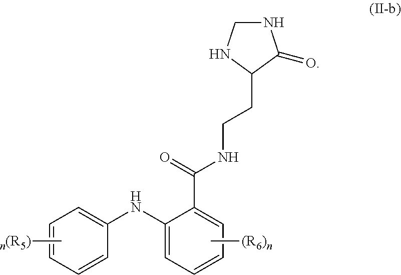

8. The compound of claim 7, wherein the compound of formula (I-b) is:

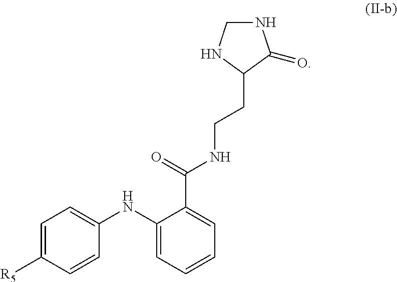

9. The compound of claim 8, wherein the compound of formula (I-b) is selected from:

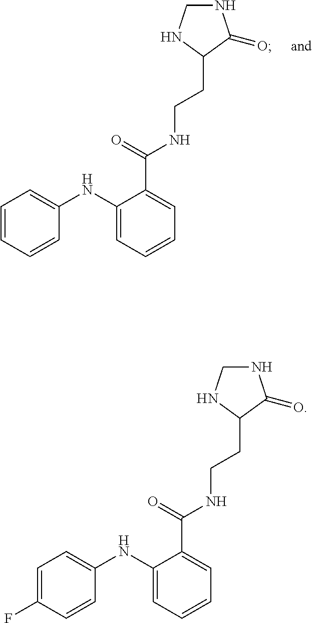 [SMILES: O=C(NCCC1NCNC1=O)c1ccccc1Nc1ccccc1 ; O=C(NCCC1NCNC1=O)c1ccccc1Nc1ccc(F)cc1]

10. A composition comprising a compound of claim 1 and a pharmaceutically acceptable carrier.

11. A method for treating a disease, condition, or disorder associated with phosphorylation of eukaryotic initiation factor 2 alpha (eIF2α), the method comprising administering to a subject in need of treatment thereof a therapeutically effective amount of a compound of formula (I) of claim 1.

12. The method of claim 11, wherein the disease, condition, or disorder associated with phosphorylation of eIF2α comprises a neurodegenerative disease.

13. The method of claim 12, wherein the neurodegenerative disease is selected from Alzheimer's disease, Parkinson disease, Creutzfeldt-Jakob disease, Huntington's disease, frontotemporal dementia (FTD), and amyotrophic lateral sclerosis (ALS).

14. The method of claim 11, wherein the administering of a therapeutically effective amount of a compound of formula (I) inhibits microtubule affinity-regulating kinase 2 (MARK2) kinase activity.

15. The method of claim 14, wherein inhibiting MARK2 kinase activity reduces phosphorylation of eukaryotic initiation factor 2 alpha (eIF2α).

16. The method of claim 15, wherein reducing the phosphorylation of eIF2α reduces the phosphorylation of eIF2α-⁵¹S.

17. The method of claim 11, wherein the phosphorylation of eIF2α is associated with a response to proteotoxic stress.

18. The method of claim 17, wherein the proteotoxic stress is associated with protein misfolding.

19. The method of claim 11, wherein phosphorylation of eIF2α is associated with regulating translation under stress.
